(19) United States
(12) Patent Application Publication
(10) Pub. No.: US 2005/0042234 A1
(43) Pub. Date: Feb. 24, 2005

(54) Method

(76) Inventors: Alan John Kingsman, Oxford (GB); Narry Kim, Philadelphia (PA); Ekaterini Kotsopoulou, Cambridge (GB); Jonathan Rohll, San Diego, CA (US); Kyriacos A. Mitrophanous, Oxford (GB)

Correspondence Address: FROMMER LAWRENCE & HAUG, 745 FIFTH AVENUE- 10TH FL., NEW YORK, NY, 10151, US

(21) Appl. No.: 10/258,089

(22) PCT Filed: Apr. 18, 2001
(86) PCT No.: PCT/GB01/01784

(30) Foreign Application Priority Data
Apr. 19, 2000 (GB) 0009760.0

(51) Int. Cl. A61K 39/21; C12N 7/00

(52) U.S. Cl. 424/208.1; 435/235.1

(57) ABSTRACT

Provided herein are methods for producing a replication defective retrovirus, which comprises transfecting a producer cell with (1) a retroviral genome; (2) a nucleotide sequence coding for retroviral gag and pol proteins that is codon-optimized for expression in the producer cell; and (3) nucleotide sequences encoding other essential viral packaging components not encoded by the nucleotide sequence of (1).

### RELATED APPLICATIONS

This application claims priority to International Application number PCT/GB01/01784, filed 18 Apr. 2001, which claims priority to Great Britain application number GB 0009760.0, filed 19 Apr. 2000. Each of these documents is incorporated by reference in its entirety.

### FIELD OF THE INVENTION

The present invention relates to methods of improving the safety of retroviral vectors capable of delivering therapeutic genes for use in gene therapy, and to novel nucleotide sequences for use in such methods.

### BACKGROUND

Retroviral vectors are now widely used as vehicles to deliver genes into cells. Their popularity stems from the fact that they are easy to produce and mediate stable integration of the gene that they carry into the genome of the target cell. This enables long-term expression of the delivered gene (Miller, N., and J. Whelan. 1997. Hum Gene Ther. 8:803-15).

There has been considerable interest, for some time, in the development of retroviral vector systems based on lentiviruses. Lentiviruses are a small subgroup of complex retroviruses. They contain, in addition to the common retroviral genes (gag, pol and env), genes which enable them to regulate their life cycle and to infect non-dividing cells (Lewis & Emerman. 1993. J. Virol. 68:510). Vector systems based thereon are therefore of interest because of their potential use in the transfer of a gene of interest to non-dividing cells such as neurones. In addition, lentiviral vectors enable very stable long-term expression of the gene of interest. This has been shown to be at least three months for transduced rat neuronal cells while MLV based vectors were only able to express the gene of interest for six weeks.

The most commonly used lentivirus is the Human Immunodeficiency Virus (HIV), the etiologic agent of AIDS (acquired immune deficiency syndrome). HIV-based vectors have been shown to efficiently transduce non-diving cells (Naldini, L., U. Blomer, P. Gallay, D. Ory, R. Mulligan, F. H. Gage, I. M. Verma, and D. Trono. 1996. Science. 272:263-7) and can be used, for example, to target anti-HIV therapeutic genes to HIV susceptible cells.

However, H IV vectors have a number of significant disadvantages that may limit their therapeutic application to certain diseases. In particular, HIV-1 is a human pathogen carrying potentially oncogenic proteins and sequences. There is the risk that introduction of vector particles produced in packaging cells which express HIV gag-pol will introduce these proteins into the patient leading to seroconversion.

Emphasis has therefore been placed on the safety of these vectors. One strategy looks at the design of production systems for retroviral vectors. A retrovirus vector system basically consists of two elements, a packaging cell line and a vector genome. The simplest packaging line consists of a provirus in which the ψ sequence (a determinant of RNA packaging reporting in HIV as lying between U5 and gag) has been deleted. When stably transfected into a cell, virus particles containing reverse transcriptase will be produced but virion RNA will not become packaged within these particles. The complementing component in a retrovirus vector system is the genome vector itself. The genome vector needs to contain a packaging sequence but much of the structural coding regions can be deleted. Often a selectable marker gene, or other nucleotide sequence of interest, is incorporated into the vector. Vector stocks of the packaging line can then be used to infect target cells. Provided the cell is successfully infected by the viral particle, the genome vector sequence will be reverse transcribed and integrated by the retroviral machinery. However, infection is an end process so no further replication or spread of the vector should occur.

As indicated above, however, problems are encountered in the design of safe and effective retroviral vectors. These include the possibility that recombination between the packaging vector and the packaging sequence can lead to the generation of wild type replication competent virus. Consequently efforts have been directed at improving the safety of packaging cell constructs.

In second generation packaging cell lines, in addition to deletion of the packaging sequence, the 3′ LTR was also deleted so that two recombinations are necessary to generate a wild type virus.

In third generation packaging lines the gag-pol genes and env gene are placed on separate constructs that are sequentially introduced into the packaging cells to prevent recombination during transfection.

With regard to the packaging signal, EP 0 368 882A (Sodroski) discloses that in HIV it corresponds to the region between the 5′ major splice donor and the gag initiation codon, and particularly corresponds to a segment just downstream of the 5′ major splice donor, and about 14 bases upstream of the gag initiation codon. It is this region which Sodroski teaches should be deleted from the gag-pol cassette. WO97/12622 (Verma) describes that in HIV-1 a 39 bp internal deletion in the ψ sequence can be made between the 5′ splice donor site and the starting codon of the gag gene.

Codon wobbling can be used to reduce recombination frequency while maintaining the primary protein sequence of the constructs, c.f. (Morgenstern & Land. 1990. Nucleic Acids Res. 18: 3587-3596) in which the region of overlap between the gag-pol and env expression constructs was reduced to 61 bp extending over the common region between pol and env which are in different reading frames. Transversion mutations were introduced into the final 20 codons of pol, retaining the integrity of the coding region while reducing the homology with env to 55% in the overlap region. Similarly wobble mutations were introduced into the 3′ of env and all sequences downstream of the env stop codon were deleted.

Efficient vectors usually contain part of gag on the genome vector to increase virion titre. Unlike the packaging sequence which can be in any position within a sequence to effect packaging, the gag sequence must be in its native position adjacent to ψ to have any effect.

It will be appreciated that whilst significant improvements in packaging cell and vector design have been made there is still scope for further refinement of current packaging lines.

### SUMMARY

It has been discovered that codon optimization of gag-pol retroviral sequences increases viral titre by overcoming potential recombination problems with vector genomes that carry part of a gag sequence. This codon optimization strategy also avoids the requirement of using gag regions from different viruses in the packaging and vector genome constructs. It was reported in WO99/41397 that codon optimization of gag-pol enhanced RNA stability and overcame the Rev/RRE requirement for export.

Also, it has been discovered that the codon optimization reported herein disrupts RNA secondary structures, such as the packaging signal, thus rendering the gag-pol mRNA non-packagable. Thus, the codon optimization described herein allows retroviral sequences upstream of the gag initiation codon to be retained without significantly compromising safety.

Thus, provided herein are retroviral particles having codon optimized gag and pol sequences that offer improved safety over the corresponding wild type viral particle. The retroviral particles often are lentiviral particles, and frequently carry nucleotide constructs that encode therapeutic proteins.

Also provided are methods of using a nucleotide sequence coding for retroviral gag and pol proteins, capable of assembly in a retroviral vector genome into a retroviral particle in a producer cell, to generate a replication defective retrovirus in a target cell, wherein the nucleotide sequence is codon optimized for expression in the producer cell.

In one embodiment, provided herein is the use of a nucleotide sequence coding for retroviral gag and pol proteins capable of assembly of a retroviral vector genome into a retroviral particle in a producer cell to reduce or prevent packaging of the retroviral vector genome in a target cell, where the nucleotide sequence is codon optimized for expression in the producer cell. The term “reducing” as used herein refers to a lower probability of an event occurring in a gag-pol optimized sequence as compared to the wild-type gag-pol sequence. Within a population of cells, the probability of an event occurring may be prevented for an individual retrovirus vector or particle.

In another embodiment, provided herein is the use of a nucleotide sequence coding for retroviral gag and pol proteins, capable of assembly of a retroviral vector genome comprising at least part of a gag nucleotide sequence into a retroviral particle in a producer cell, to reduce or prevent recombination between the nucleotide sequence coding for retroviral gag and pol proteins and at least part of a gag nucleotide sequence, where the nucleotide sequence coding for retroviral gag and pol proteins is codon optimized for expression in the producer cell.

In a specific embodiment, provided herein is a method of producing a replication defective retrovirus comprising transfecting a producer cell with the following: (i) a retroviral genome; (ii) a nucleotide sequence coding for retroviral gag and pol proteins; and (iii) nucleotide sequences encoding other essential viral packaging components not encoded by the nucleotide sequence of (ii); where the nucleotide sequence coding for retroviral gag and pol proteins is codon optimized for expression in the producer cell.

In another embodiment, provided herein is a method of reducing or preventing packaging of a retroviral genome in a target cell comprising the steps of: a. transfecting a producer cell with the following to produce retroviral particles: (i) a retroviral genome; (ii) a nucleotide sequence coding for retroviral gag and pol proteins; and (iii) nucleotide sequences encoding other essential viral packaging components not encoded by one or more of the nucleotide sequences of (ii); and b. transfecting a target cell with retroviral particles of step (a); where the nucleotide sequence coding for retroviral gag and pol proteins is codon optimized for expression in the producer cell.

In yet another embodiment, provided herein is a method to reduce or prevent recombination between a retroviral vector genome and a nucleotide sequence encoding a viral polypeptide required for the assembly of the viral genome into retroviral particles comprising transfecting a producer cell with the following: (i) a retroviral genome comprising at least part of a gag nucleotide sequence; (ii) a nucleotide sequence coding for retroviral gag and pol proteins; and (iii) nucleotide sequences encoding other essential viral packaging components not encoded by the nucleotide sequence of (ii); where the nucleotide sequence coding for retroviral gag and pol proteins is codon optimized for expression in the producer cell.

Also provided are codon-optimized gag-pol sequences presented herein as SEQ ID NO:15 and and SEQ ID NO:16. It should be appreciated, however, that any convenient codon optimized gag-pol sequence may be utilized in methods described herein.

Further, provided herein are retroviral particles produced using the optimized sequences described, and methods of producing such retroviral particles. Also provided are pharmaceutical compositions which comprise a viral particle described herein, together with a pharmaceutically acceptable diluent or carrier.

### BRIEF DESCRIPTION OF THE DRAWINGS

FIGS. 1A and 1B show schematically how to create a suitable 3′ LTR by PCR.

FIG. 2 shows the codon usage table for wild type HIV gag-pol of strain HXB2 (accession number: K03455).

FIG. 3A shows the codon usage table of the codon optimized sequence designated gagpol-SYNgp. FIG. 3B shows a comparative codon usage table.

FIG. 4 shows the codon usage table of the wild type HIV env called env-mn.

FIG. 5 shows the codon usage table of the codon optimized sequence of HIV env designated SYNgp160 nm.

FIG. 6 shows two plasmid constructs for use in the invention.

FIG. 7 shows the principle behind two systems for producing retroviral vector particles.

FIG. 8 shows a sequence comparison between the wild type HIV gag-pol sequence (pGP-RRE3) and the codon optimized gag-pol sequence (pSYNGP). FIG. 8 shows a sequence comparison between the wild type HIV gag-pol sequence (pGP-RRE3) and the codon optimized gag-pol sequence (pSYNGP) wherein the upper sequence represents pSYNGP and the lower sequence represents pGP-RRE3.

FIG. 9 shows a sequence comparison between the wild type EIAV gag-pol sequence (WT) and the codon optimized gag-pol sequence (CO).

FIGS. 10A-10C show Rev independence of protein expression particle formation. 5 μg of the gag-pol expression plasmids were transfected into 293T cells in the presence or absence of Rev (pCMV-Rev, 1 μg) and protein levels were determined 48 hours post transfection in culture supernatants (A) and cell lysates (B). HIV-1 positive human serum was used to detect the gag-pol proteins. The blots were re-probed with an anti-actin antibody, as an internal control (C). The protein marker (New England Biolabs) sizes (in kDa) are shown on the side of the gel. Lanes in the gel are: 1. Mock transfected 293T cells, 2. pGP-RRE3, 3. pGP-RRE3+pCMV-Rev, 4. pSYNGP, 5. pSYNGP+pCMV-Rev, 6. pSYNGP-RRE, 7. pSYNGP-RRE+pCMV-Rev, 8. pSYNGP-ERR, 9. pSYNGP-ERR+pCMV-Rev.

FIGS. 11A-11C show translation rates of wild-type (WT) and codon optimized gag-pol. 293T cells were transfected with 2 μg pGP-RRE3 (+/−1 μg pCMV-Rev) or 2 μg pSYNGP. Protein samples from culture supernatants (A) and cell extracts (B) were analysed by Western blotting 12, 25, 37 and 48 hours post-transfection. HIV-1 positive human serum was used to detect gag-pol proteins (A, B) and an anti-actin antibody was used as an internal control (C). The protein marker sizes are shown on the side of the gel (in kD). A Phosphorimager was used for quantification of the results. Lanes are: 1. pGP-RRE3 12 h, 2. pGP-RRE3 25 h, 3. pGP-RRE3 37 h, 4. pGP-RRE3 48 h, 5. pGP-RRE3+pCMV-Rev 12 h, 6. pGP-RRE3+pCMV-Rev 25 h, 7. pGP-RRE3+pCMV-Rev 37 h, 8. pGP-RRE3+pCMV-Rev 48 h, 9. pSYNGP 12 h, 10. pSYNGP 25 h, 11. pSYNGP 37 h, 12. pSYNGP 48 h, 13. Mock transfected 293 T cells.

FIGS. 12A and 12B show gag-pol mRNA levels in total and cytoplasmic fractions. Total and cytoplasmic RNA was extracted from 293T cells 36 hours after transfection with 5 μg of the gag-pol expression plasmid (+/−1 μg pCMV-Rev) and mRNA levels were estimated by Northern blot analysis. A probe complementary to nt 1222-1503 of both the wild type and codon optimized gene was used. Panel A shows the band corresponding to the HIV-1 gag-pol. The sizes of the mRNAs are 4.4 kb for the codon optimized and 6 kb for the wild type gene. Panel B shows the band corresponding to human ubiquitin (internal control for normalisation of results). Quantification was performed using a Phosphorimager. Lane numbering: c indicates cytoplasmic fraction and t indicates total RNA fraction. Lanes: 1. pGP-RRE3, 2. pGP-RRE3+pCMV-Rev, 3. pSYNGP, 4. pSYNGP+pCMV-Rev, 5. pSYNGP-RRE, 6. pSYNGP-RRE+pCMV-Rev, 7. Mock transfected 293T cells, 8. pGP-RRE3+pCMV-Rev, 9. Mock transfected 293T cells, 10. pSYNGP.

FIGS. 13A and 13B show the effect of insertion of WT gag downstream of the codon optimized gene on RNA and protein levels. The wt gag sequence was inserted downstream of the codon optimized gene in both orientations (NotI site), resulting in plasmids pSYN6 (correct orientation, see FIG. 14) and pSYN7 (reverse orientation, see FIG. 14). The gene encoding for β-galactosidase (LacZ) was also inserted in the same site and the correct orientation (plasmid pSYN8, see FIG. 14). 293T cells were transfected with 5 μg of each plasmid and 48 hours post transfection mRNA and protein levels were determined as previously described by means of Northern and Western blot analysis respectively. For the, Northern blot analysis of cytoplasmic RNA fractions, the blot was probed with a probe complementary to nt 1510-2290 of the codon optimized gene (I) and was re-probed with a probe specific for human ubiquitin (II). Lanes: 1. pSYNGP, 2. pSYN8, 3. pSYN7, 4. pSYN6. For the, Western blot analysis, HIV-1 positive human serum was used to detect the gag-pol proteins (I) and an anti-actin antibody was used as an internal control (II). Lanes: Cell lysates: 1. Mock transfected 293T cells, 2. pGP-RRE3+pCMV-Rev, 3. pSYNGP, 4. pSYN6, 5. pSYN7, 6. pSYN8. Supernatants: 7. Mock transfected 293T cells, 8. pGP-RRE3+pCMV-Rev, 9. pSYNGP, 10. pSYN6, 11. pSYN7, 12. pSYN8. The protein marker (New England Biolabs) sizes are shown on the side of the gel.

FIG. 14 shows the plasmids used to study the effect of HIV-1 gag on the codon optimized gene. The backbone for all constructs was pCI-Neo. Syn gp: The codon optimized HIV-1 gag-pol gene. HXB2 gag: The wild type HIV-1 gag gene. HXB2 gag r: The wild type HIV-1 gag gene in the reverse orientation. HXB2 gagΔATG: The wild type HIV-1 gag gene without the gag ATG. HXB2 gag-fr.sh.: The wild type HIV-1 gag gene with a frameshift mutation. HXB2 gag 625-1503: Nucleotides 625-1503 of the wild type HIV-1 gag gene. HXB2 gag 1-625: Nucleotides 1-625 of the wild type HIV-1 gag gene.

FIGS. 15A and 15B show the effect on cytoplasmic RNA of insertion of HIV-1 gag upstream of the codon optimized gene. Cytoplasmic RNA was extracted 48 hours post transfection of 293T cells (5 μg of each pSYN plasmid was used and 1 μg of pCMV-Rev was co-transfected in some cases). The probe that was used was designed to be complementary to nt 1510-2290 of the codon optimized gene (I). A probe specific for human ubiquitin was used as an internal control (II). Lanes in FIG. 15A are: 1. pSYNGP, 2. pSYN9, 3. pSYN10, 4. pSYN10+pCMV-Rev, 5. pSYN11, 6. pSYN11+pCMV-Rev, 7. pCMV-Rev. Lanes in FIG. 15B are: 1. pSYNGP, 2. pSYNGP-RRE, 3. pSYNGP-RRE+pCMV-Rev, 4. pSYN12, 5. pSYN14, 6. pSYN14+pCMV-Rev, 7. pSYN13, 8. pSYN15, 9. pSYN17, 10. pGP-RRE3, 11. pSYN6, 12. pSYN9, 13. pCMV-Rev.

FIGS. 16A and 16B show the effect of Leptomycin B (LMB) on protein production. 293T cells were transfected with 1 μg pCMV-Rev and 3 μg of pGP-RRE3/pSYNGP/pSYNGP-RRE (+/−1 μg pCMV-Rev). Transfections were done in duplicate. 5 hours post transfection the medium was replaced with fresh medium in the first set and with fresh medium containing 7.5 nM LMB in the second. 20 hours later the cells were lysed and protein production was estimated by Western blot analysis. HIV-1 positive human serum was used to detect the gag-pol proteins (A) and an anti-actin antibody was used as an internal control (B). Lanes: 1. pGP-RRE3, 2. pGP-RRE3+LMB, 3. pGP-RRE3+pCMV-Rev, 4. pGP-RRE3+pCMV-Rev+LMB, 5. pSYNGP, 6. pSYNGP+LMB, 7. pSYNGP+pCMV-Rev, 8. pSYNGP+pCMV-Rev+LMB, 9. pSYNGP-RRE, 10. pSYNGP-RRE+LMB, 11. pSYNGP-RRE+pCMV-Rev, 12. pSYNGP-RRE+pCMV-Rev+LMB.

FIG. 17 shows the cytoplasmic RNA levels of the vector genomes. 293T cells were transfected with 10 μg of each vector genome. Cytoplasmic RNA was extracted 48 hours post transfection. 20 μg of RNA were used from each sample for Northern blot analysis. The 700 bp probe was designed to hybridise to all vector genome RNAs (see Materials and Methods). Lanes: 1. pH6nZ, 2. pH6nZ+pCMV-Rev, 3. pH6.1nZ, 4. pH6.1nZ+pCMV-Rev, 5. pHS1nZ, 6. pHS2nZ, 7. pHS3nZ, 8. pHS4nZ, 9. pHS5nZ, 10. pHS6nZ, 11. pHS7nZ, 12. pHS8nZ, 13. pCMV-Rev.

FIG. 18 shows transduction efficiency at MOI 1. Viral stocks were generated by co-transfection of each gag-pol expression plasmid (5 or 0.5 μg), 15 μg pH6nZ or pHS3nZ (vector genome plasmid) and 5 μg pHCMVG (VSV envelope expression plasmid) on 293T cells. Virus was concentrated as previously described (Zhu, Z. H., S. S. Chen, and A. S. Huang. 1990. J Acquir Immune Defic Syndr. 3:215-9) and transduction efficiency was determined at m.o.i.'s 0.01-1 on HT1080 cells. There was a linear correlation of transduction efficiency and m.o.i. in all cases. An indicative picture at m.o.i. 1 is shown here. Transduction efficiency was >80% with either genome, either gag-pol and either high or low amounts of pSYNGP. Titres before concentration (I.U./ml): on 293T cells: A. 6.6×10⁵, B. 7.6×10⁵, C. 9.2×10⁵, D. 1.5×10⁵, on HT1080 cells: A. 6.0×10⁴, B. 9.9×10⁴, C. 8.0×10⁴, D. 2.9×10⁴. Titres after concentration (I.U./ml) on HT1080 cells: A. 6.0×10⁵, B. 2.0×10⁶, C. 1.4×10⁶, D. 2.0×10⁵.

FIG. 19 shows a schematic representation of pGP-RRE3.

FIG. 20 shows a schematic representation of pSYNGP.

FIG. 21 shows vector titres generated with different gag-pol constructs. Viral stocks were generated by co-transfection of each gag-pol expression plasmid, pH6nZ (vector genome plasmid) and pHCMVG (VSV envelope expression plasmid, 2.5 μg for each transfection) on 293T cells. Titres (I.U./ml of virs stock) were measured on 293T cells by counting the number of blue colonies following X-Gal staining 48 hours after transduction. Experiments were performed at least twice and the variation between experiments was less than 15%.

FIG. 22 shows vector titres from the Rev/RRE (−) and (+) genomes. The retroviral vectors were generated as described in the Examples. Titres (I.U./ml of viral stock+SD) were determined in 293T cells.

FIG. 23 shows vector titres from the pHS series of vector genomes. The retroviral vector was generated as described in the Examples. Titres (I.U./ml of viral stock+SD) were determined in 293T cells. Rev is provided from pCMV-Rev. Note that pH6nZ expresses Rev and contains the RRE. None of the other genomes express Rev or contain the RRE. Expression from pSYNGP is Rev independent, whereas it is Rev dependent for pGP-RRE3.

FIG. 24 shows vector titres for the pHS series of vector genomes in the presence or absence of Rev/RRE. The retroviral vector was generated as described in the Examples. 5 μg of vector genome, 5 μg of pSYNGP and 2.5 μg of pHCMVG were used and titres (I.U./ml) were determined in 293T cells. Experiments were performed at least twice and the variation between experiments was less than 15%. Rev is provided from pCMV-Rev (1 μg). Note that pH6nZ expresses Rev and contains the RRE. None of the pHS genomes expresses Rev and only pHS1nZR, pHS3nZR, pHS7nZR and pH6.1nZR contain the RRE. gag-pol expression from pSYNGP is Rev independent.

FIG. 25 shows an analysis of gag-pol constructs.

FIG. 26 shows a Western blot of 293T extracts. FIG. 26 shows a Western blot of 293T extracts wherein 30:g of total cellular protein was separated by SDS/Page electrophoresis, transferred to nitro-cellulose and probed with anti EIAV antibodies. The secondary antibody was anti-Horse HRP (Sigma).

FIG. 27 is a schematic representation of pESYNGP.

FIG. 28 is a schematic representation of LpESYNGP.

FIG. 29 is a schematic representation of LpESYNGPRRE.

FIG. 30 is a schematic representation of pESYNGPRRE.

FIG. 31 is a schematic representation of pONY4.0Z.

FIG. 32 is a schematic representation of pONY8.0Z.

FIG. 33 is a schematic representation of pONY8.1Z.

FIG. 34 is a schematic representation of pONY3.1.

FIG. 35 is a schematic representation of pCIneoERev.

FIG. 36 is a schematic representation of pESYNREV.

FIGS. 37 and 38 show the effect of different vector constructs on viral vector titres. In FIG. 38 the titres are shown in lacZ forming units (L.F.U.)/ml. The vectors used are indicated in boxes above the bars.

FIGS. 39 and 40 show the effect of different vector constructs on RT activity.

FIG. 41 shows the effect of the 5′ leader sequence on viral vector titre.

FIG. 42 shows viral vector titres when using pONY8.1Z.

FIG. 43 shows a comparison between the sequences of pONY3.1 and codon optimized pONY3.2OPTI in the first 372 nucleotides of gag.

FIG. 44 is a schematic representation of pIRES1hygESYNGP.

FIGS. 45 and 46 show the results of experiments to confirm that codon optimized gag-pol can be used in the production of packaging and producer cell lines.

FIGS. 47, 48A and 48B show the results of experiments which confirm that RNA from codon optimized gag-pol is packaged less efficiently than that from the wild-type gene.

FIG. 49 shows the results of an experiment which confirms that expression from pESYNGP and pESDSYNGP are similar.

FIG. 50 is a schematic representation of pESDSYNGP.

FIG. 51 shows the results of an experiment which confirms that the efficiency of encapsidating gag-pol RNA in PEV-17 cells and B-241 cells in similar.

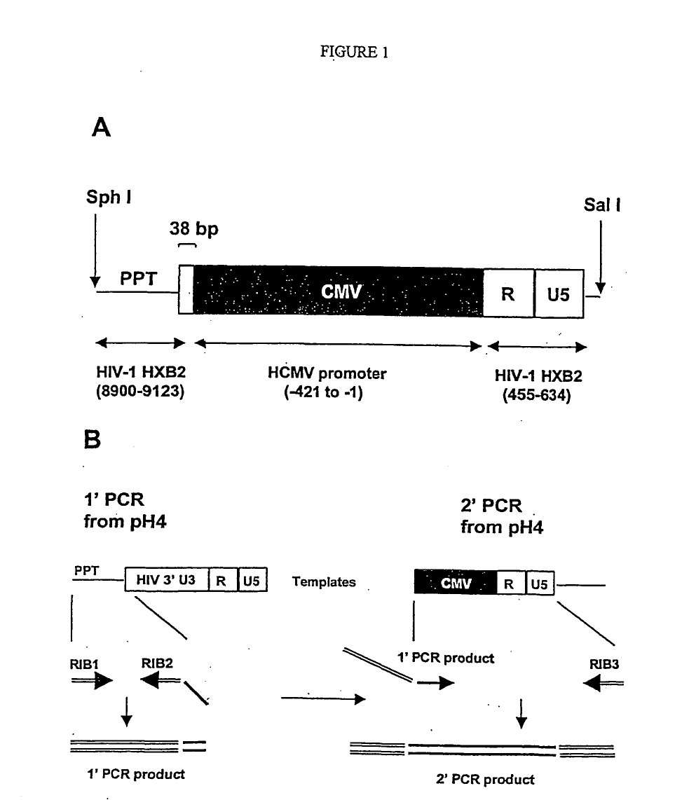

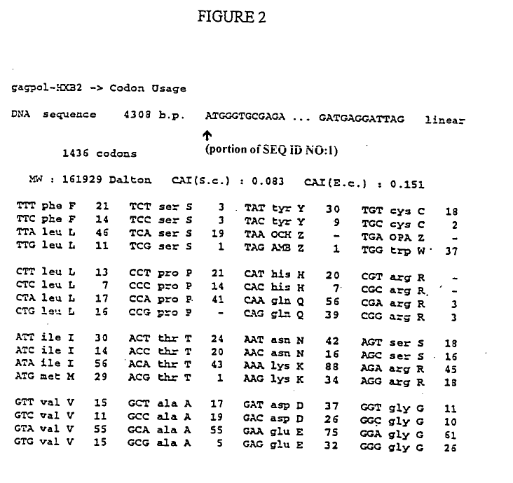

### BRIEF DESCRIPTION OF THE SEQUENCES

SEQ ID NO:1 shows the sequence of the wild-type gag-pol sequence for the strain HXB2 (accession no. K03455);
SEQ ID NO:2 shows the sequence of pSYNGP;
SEQ ID NO:3 shows the sequence of the Envelope gene for HIV-1 MN (Genbank accession no. M17449);
SEQ ID NO:4 shows the sequence of SYNgp-160 nm-codon optimized env sequence;
SEQ ID NO:5 shows the sequence of pESYNGP;
SEQ ID NO:6 shows the sequence of LpESYNGP;
SEQ ID NO:7 shows the sequence of pESYNGPRRE;
SEQ ID NO:8 shows the sequence of LpESYNGPRRE;
SEQ ID NO:9 shows the sequence of pONY4.0Z;
SEQ ID NO:10 shows the sequence of pONY8.0Z;
SEQ ID NO:11 shows the sequence of pONY8.1 Z;
SEQ ID NO:12 shows the sequence of pONY3.1;
SEQ ID NO:13 shows the sequence of pCIneoERev;
SEQ ID NO:14 shows the sequence of pESYNREV;
SEQ ID NO:15 shows the sequence of codon optimized HIV gag-pol;
SEQ ID NO:16 shows the sequence of codon optimized EIAV gag-pol;
SEQ ID NO:17 shows the sequence of pIRES1hygESYNGP;
SEQ ID NO:18 shows the sequence of pESDSYNGP; and
SEQ ID NO:19 shows the sequence of pONY8.3G FB29(−).

### DETAILED DESCRIPTION

Various features and embodiments of the present invention will now be described by way of non-limiting example. The present invention employs the concept of codon optimization.

Codon optimization has previously been described in WO99/41397 as a means of overcoming the Rev/RRE requirement for export and to enhance RNA stability. The alterations to the coding sequences for the viral components improve the sequences for codon usage in the mammalian cells or other cells which are to act as the producer cells for retroviral vector particle production. This improvement in codon usage is referred to as “codon optimization”. Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms.

By virtue of alterations in their sequences, nucleotide sequences encoding packaging components of the viral particles required for assembly of viral particles in the producer cells/packaging cells have RNA instability sequences (INS) eliminated from them. At the same time, the amino acid coding sequence for the packaging components is retained so that the viral components encoded by the sequences remain the same, or at least sufficiently similar that the function of the packaging components is not compromised.

The term “viral polypeptide required for the assembly of viral particles” refers to a polypeptide normally encoded by the viral genome to be packaged into viral particles, in the absence of which the viral genome cannot be packaged. For example, in the context of retroviruses such polypeptides would include gag-pol and env. The term “packaging component” is also included within this definition.

As discussed in WO99/32646, the sequence requirements for packaging HIV vector genomes are complex. The HIV-1 packaging signal encompasses the splice donor site and contains a portion of the 5′-untranslated region of the gag gene, which has a putative secondary structure containing 4 short stem-loops. However, additional sequences elsewhere in the genome are also known to be important for efficient encapsidation of HIV. For example, the first 350 bps of the gag protein coding sequence may contribute to efficient packaging. Thus, for construction of HIV-1 vectors capable of expressing heterologous genes, a packaging signal extending to 350 bps of the gag protein-coding region has been used on the vector genome. It was found that codon optimization of the gag coding region on the packaging vector, at least in the region into which the packaging signal extends, also has the effect of disrupting packaging of the vector genome. Thus codon optimization is a novel method of obtaining a replication defective viral particle.

Also, as disclosed in WO99/32646 the structure of the packaging signal in equine lentiviruses is different from that of HIV. Instead of a short sequence of 4 stem loops together with a packaging signal extending to 350 bps of the gag protein-coding region, it was found that in equine lentiviruses the packaging signal may not extend as far into the gag protein-coding region as may have been thought.

In one embodiment only codons relating to the packaging signal are codon optimized. Thus, in one embodiment, codon optimization extends to at least the first 350 bps of the gag protein coding region. In equine lentiviruses, at least, codon optimization extends to at least nucleotide 300 of the gag coding region, often to at least nucleotide 150 of the gag coding region. Although not optimal, codon optimization could extend to, say, only the first 109 nucleotides of the gag coding region. It may also be possible for codon optimization to extend to only the first codon of the gag coding region. However, the sequences often are codon optimized in their entirety, with the exception of the sequence encompassing the frameshift site.

The gag-pol gene comprises two overlapping reading frames encoding gag and pol proteins respectively. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames sometimes is not codon optimized. Retaining this fragment will enable more efficient expression of the gag-pol proteins.

For EIAV the beginning of the overlap has been taken to be nt 1262 (where nucleotide 1 is the A of the gag ATG). The end of the overlap is at 1461 bp. In order to ensure that the frameshift site and the gag, gag-pol overlap the wild type sequence has been retained from nt 1156 to 1465. This can be seen in FIG. 9b.

Derivations from optimal codon usage may be made, for example, in order to accommodate convenient restriction sites, and conservative amino acid changes may be introduced into the gag-pol proteins.

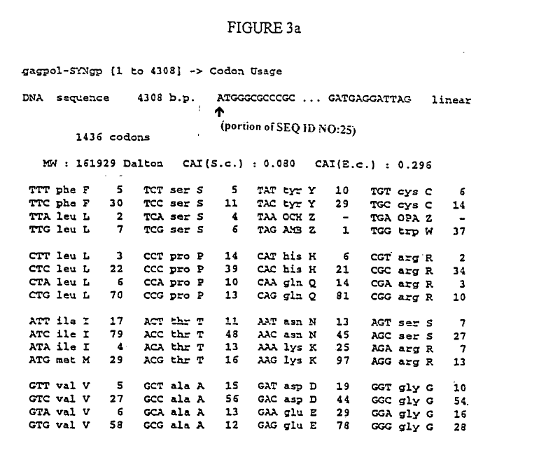

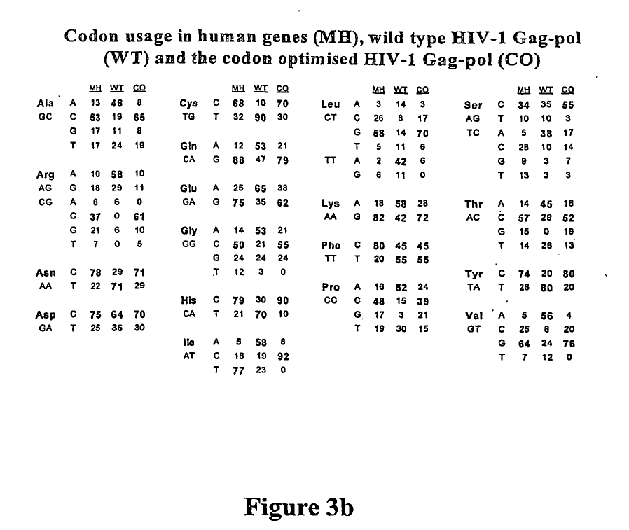

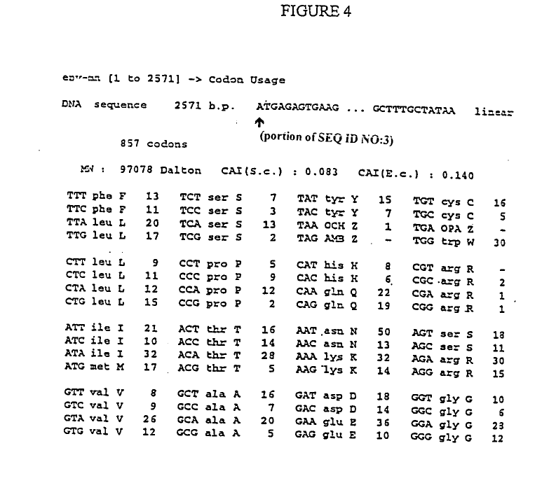

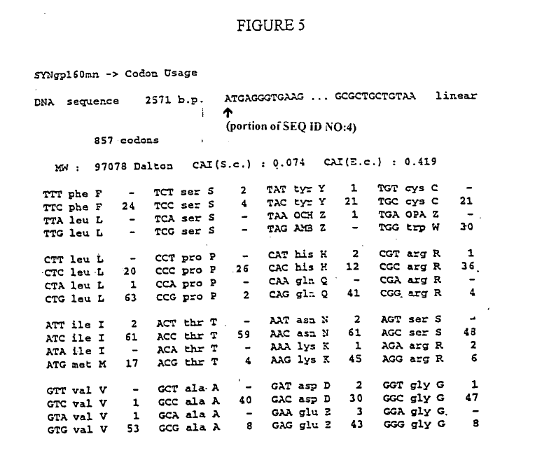

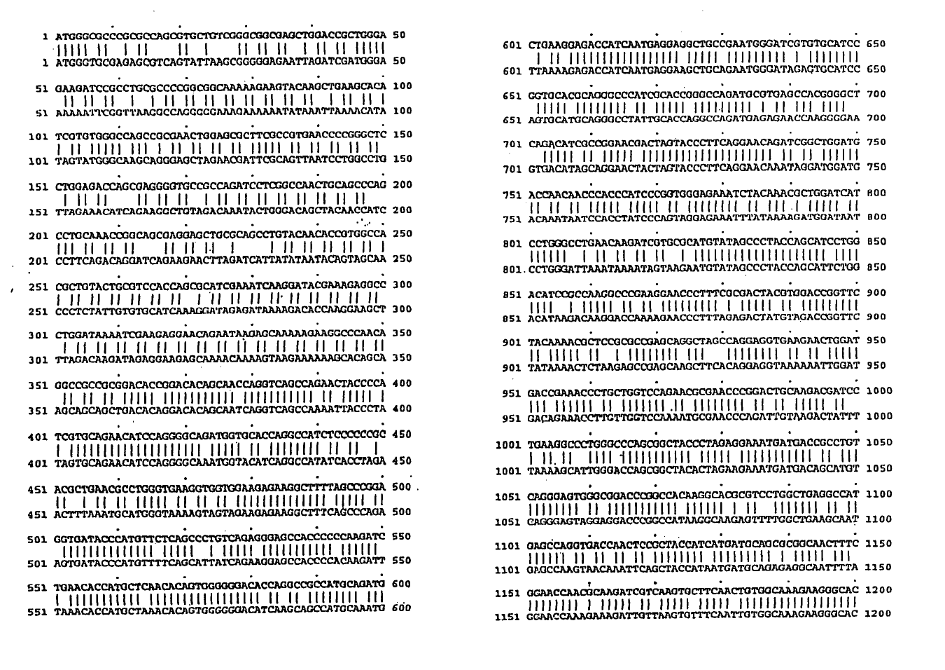

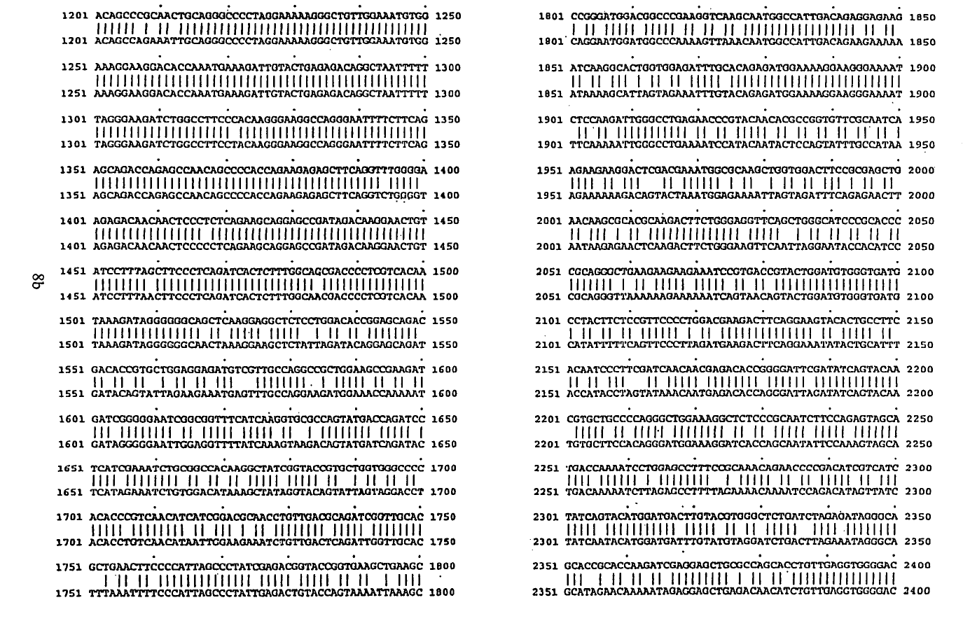

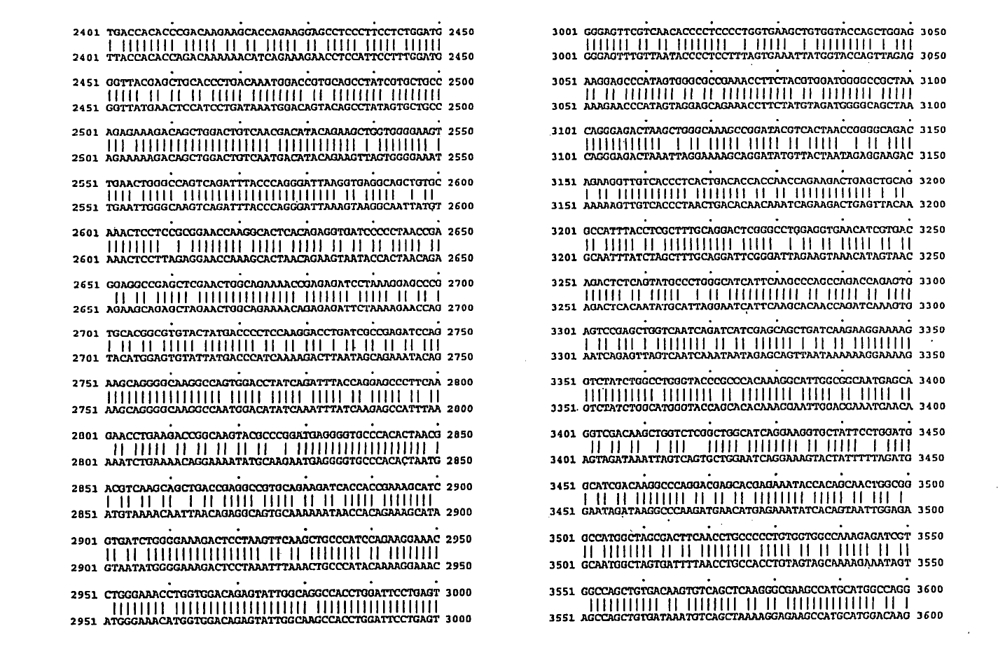

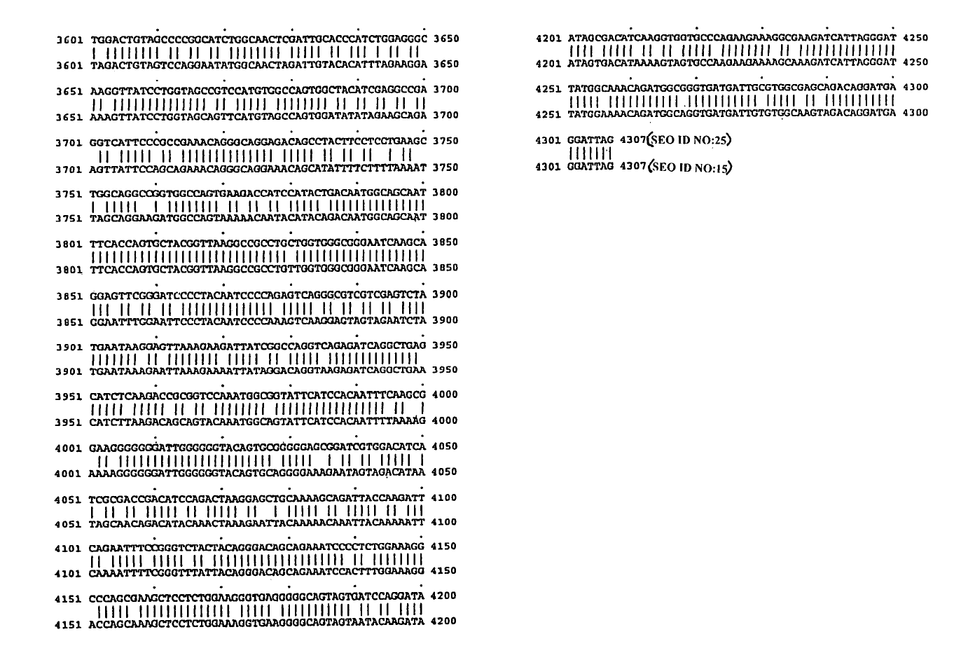

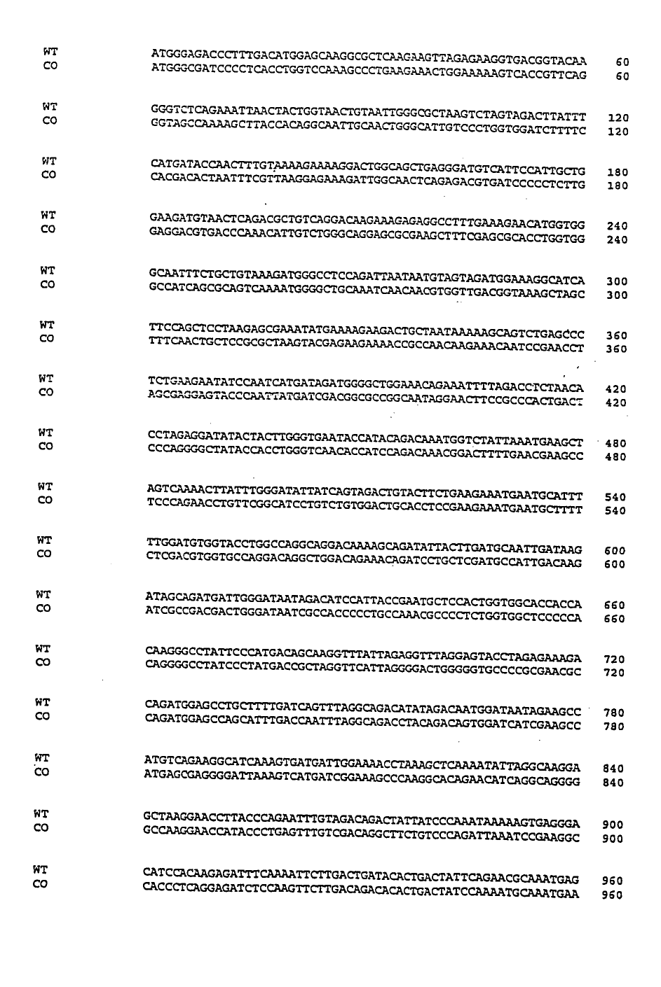

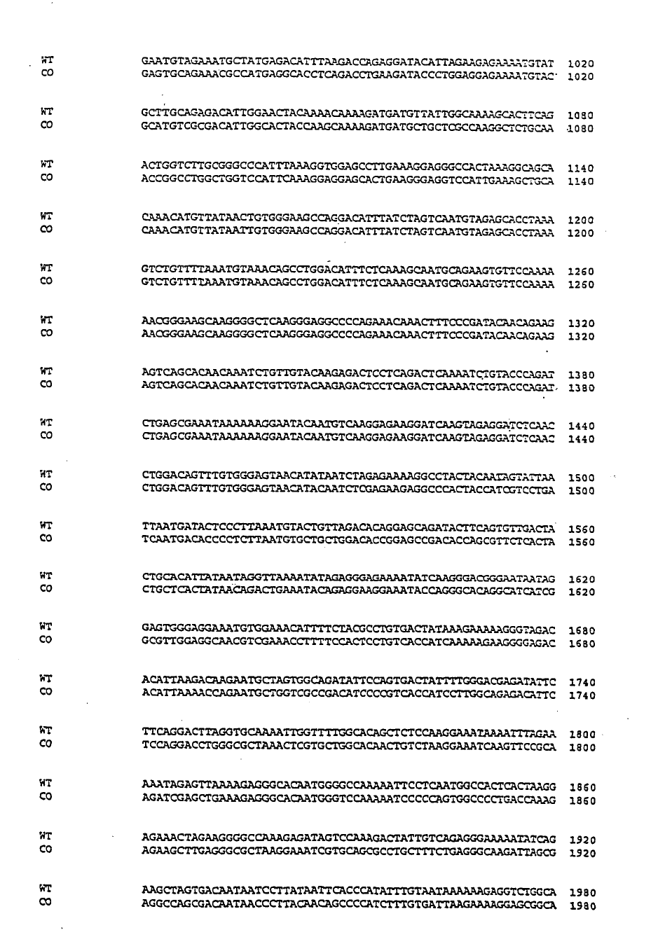

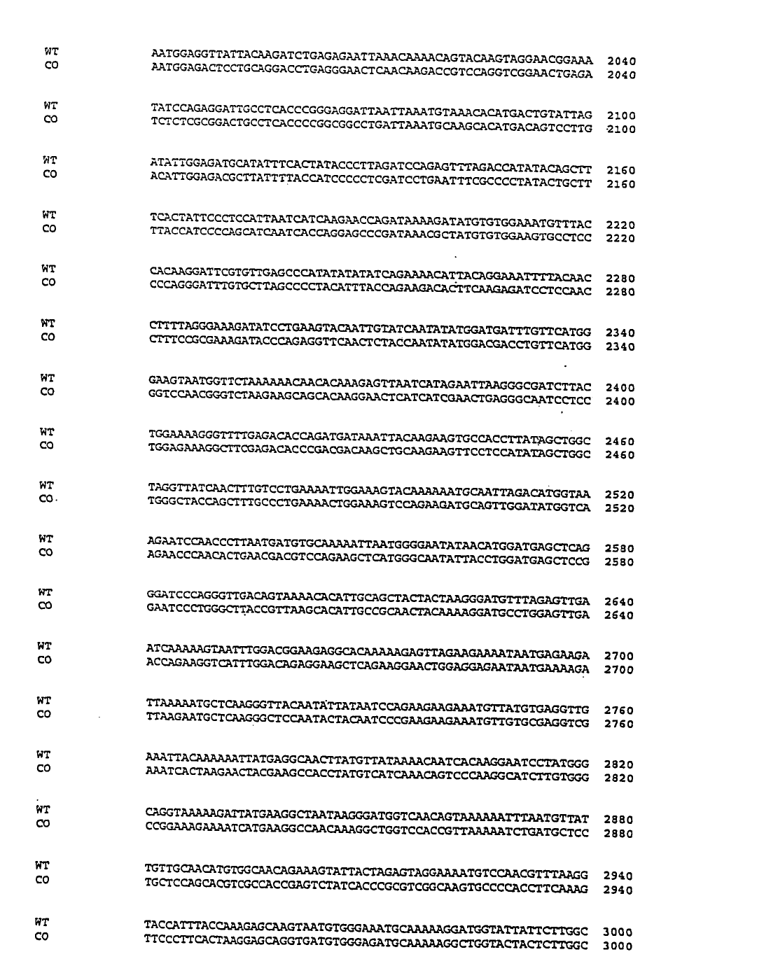

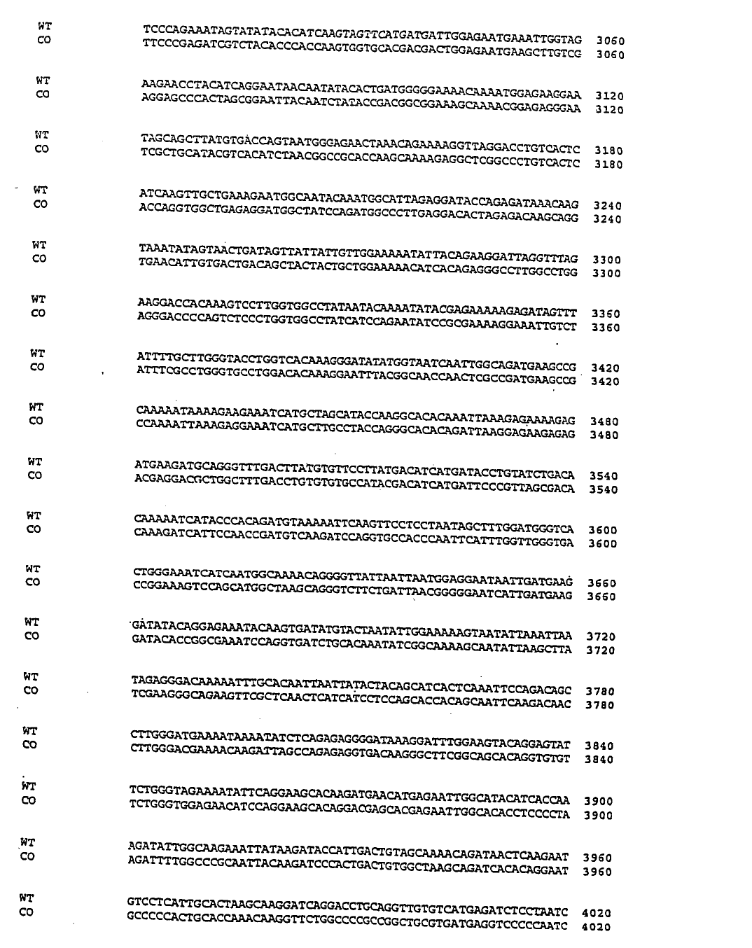

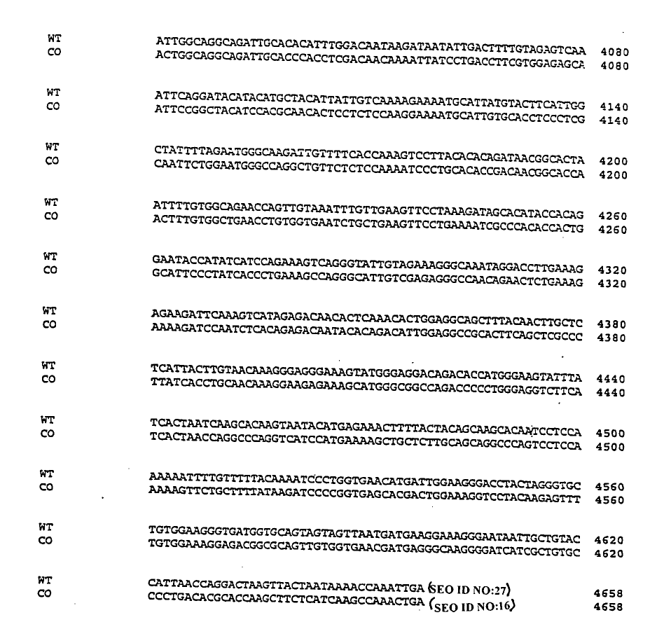

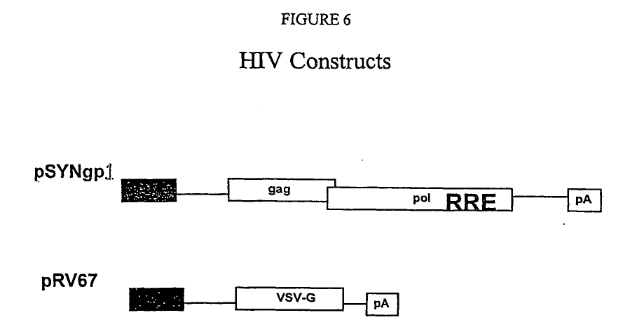

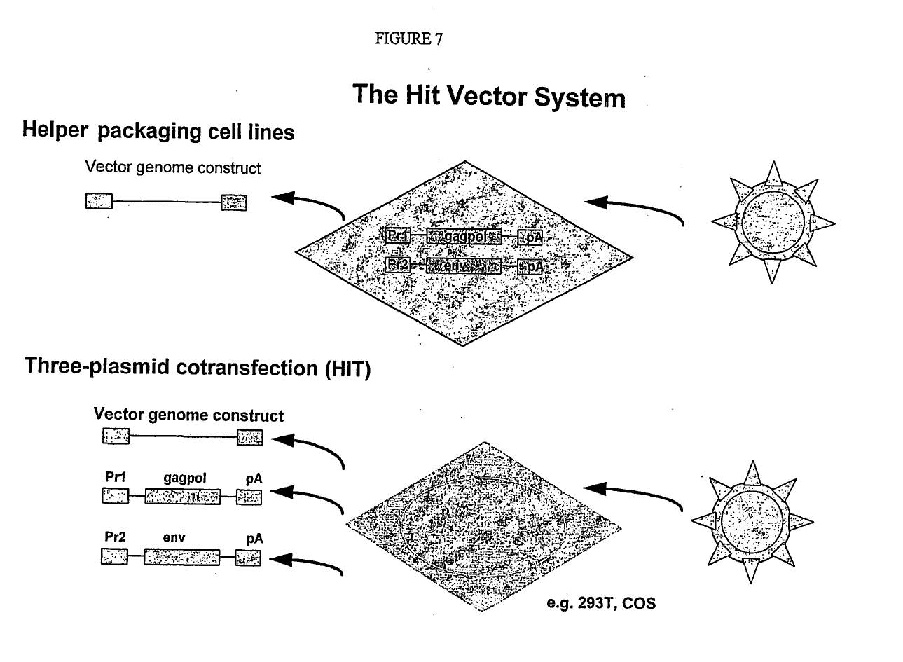

In an embodiment, codon optimization was based on lightly expressed mammalian genes. The third and sometimes the second and third base may be changed. An example of a codon usage table is given in FIG. 3b.

Due to the degenerate nature of the Genetic Code, it will be appreciated that numerous gag-pol sequences can be achieved by the skilled artisan. Also there are many retroviral variants described and which can be used as a starting point for generating a codon optimized gag-pol sequence. Lentiviral genomes can be quite variable. For example there are many quasi-species of HIV-1 which are still functional. This is also the case for EIAV. These variants may be used to enhance particular parts of the transduction process. Examples of HIV-1 variants may be found at the http address hiv-web.lanl.gov. Details of EIAV clones may be found at the NCBI database located at the http address www.ncbi.nlm.nih.gov.

The strategy for codon optimized gag-pol sequences can be used in relation to any retrovirus. This would apply to all the lentiviruses, including EIAV, FIV, BIV, CAEV, VMR, SIV, HIV-1 and HIV-2. In addition this method could be used to increase expression of genes from HTLV-1, HTLV-2, HFV, HSRV and human endogenous retroviruses (HERV).

As codon optimization may result in disruption of RNA secondary structures such as the packaging signal, it will be appreciated that any endogenous packaging signal upstream of the gag initiation codon could be retained without compromising safety.

An additional advantage of codon optimizing packaging components is that this can increase gene expression. In particular, it can render gag-pol expression Rev independent. In order to enable the use of anti-rev or RRE factors in the retroviral vector, however, it would be necessary to render the viral vector generation system totally Rev/RRE independent (Chang, D. D., and P. A. Sharp. 1989. Cell. 59:789-795). Thus, the genome also needs to be modified. This is achieved by optimizing vector genome components. Advantageously, these modifications also lead to the production of a safer system absent of all accessory proteins both in the producer and in the transduced cell, and are described below.

As described above, the packaging components for a retroviral vector include expression products of gag, pol and env genes. In addition, efficient packaging depends on a short sequence of 4 stem loops followed by a partial sequence from gag and env (the “packaging signal”). Thus, inclusion of a deleted gag sequence in the retroviral vector genome (in addition to the full gag sequence on the packaging construct) will optimize vector titre. To date, efficient packaging has been reported to require from 255 to 360 nucleotides of gag in vectors that still retain env sequences, or about 40 nucleotides of gag in a particular combination of splice donor mutation, gag and env deletions. It was surprisingly discovered that a deletion of up to 360 nucleotides in gag leads to an increase in vector titre. Further deletions resulted in lower titres. Additional mutations at the major splice donor site upstream of gag were found to disrupt packaging signal secondary structure and therefore lead to decreased vector titre. Thus, in an embodiment the retroviral vector genome includes a gag sequence from which up to 360 nucleotides have been removed.

We therefore allow the prepartion of a so-called “minimal” system in which one or more or all of the accessory genes are removed. In HIV these accessory genes are vpr, vif, tat, nef, vpu and rev. Similarly, in other lentiviruses the analogous accessory genes normally present in the lentivirus may be removed. Related embodiments also extend to systems, particles and vectors in which one or more of these accessory genes is present and in any combination.

The term “viral vector” refers to a nucleotide construct comprising a viral genome capable of being transcribed in a host cell, which genome comprises sufficient viral genetic information to allow packaging of the viral RNA genome, in the presence of packaging components, into a viral particle capable of infecting a target cell. Infection of the target cell includes reverse transcription and integration into the target cell genome, where appropriate for particular viruses. The viral vector in use typically carries heterologous coding sequences (nucleotides of interest or “NOIs”) which are to be delivered by the vector to the target cell, for example a first nucleotide sequence encoding a ribozyme. The term “replication defective” refers to a viral vector is incapable of independent replication to produce infectious viral particles within the final target cell.

The term “viral vector system” is intended to mean a kit of parts which can be used when combined with other necessary components for viral particle production to produce viral particles in host cells. For example, an NOI may typically be present in a plasmid vector construct suitable for cloning the NOI into a viral genome vector construct. When combined in a kit with a further nucleotide sequence, which will also typically be present in a separate plasmid vector construct, the resulting combination of plasmid containing the NOI and plasmid containing the further nucleotide sequence comprises the essential elements of the invention. Such a kit may then be used by the skilled person in the production of suitable viral vector genome constructs which when transfected into a host cell together with the plasmid containing the further nucleotide sequence, and optionally nucleic acid constructs encoding other components required for viral assembly, will lead to the production of infectious viral particles. Alternatively, the further nucleotide sequence may be stably present within a packaging cell line that is included in the kit.

The kit may include other components needed to produce viral particles, such as host cells and other plasmids encoding essential viral polypeptides required for viral assembly. By way of example, the kit may contain (i) a plasmid containing an NOI and (ii) a plasmid containing a further nucleotide sequence encoding a modified retroviral gag-pol construct which has been codon optimized for expression in a producer of choice. Optional components would then be (a) a retroviral genome construct with suitable restriction enzyme recognition sites for cloning the NOI into the viral genome, optionally with at least a partial gag sequence; (b) a plasmid encoding a VSV-G env protein. Alternatively, nucleotide sequence encoding viral polypeptides required for assembly of viral particles may be provided in the kit as packaging cell lines comprising the nucleotide sequences, for example a VSV-G expressing cell line.

The term “viral vector production system” refers to the viral vector system described above wherein the NOI has already been inserted into a suitable viral vector genome.

In the present invention, several terms are used interchangeably. Thus, “virion”, “virus”, “viral particle”, “retroviral particle”, “retrovirus”, and “vector particle” mean virus and virus-like particles that are capable of introducing a nucleic acid into a cell through a viral-like entry mechanism. Such vector particles can, under certain circumstances, mediate the transfer of NOIs into the cells they infect. A retrovirus is capable of reverse transcribing its genetic material into DNA and incorporating this genetic material into a target cell's DNA upon transduction. Such cells are designated herein as “target cells”.

As used herein the term “target cell” simply refers to a cell which the regulated retroviral vector of the present invention, whether native or targeted, is capable of infecting or transducing.

A lentiviral vector particle according to the invention will be capable of transducing cells which are slowly-dividing, and which non-lentiviruses such as MLV would not be able to efficiently transduce. Slowly-dividing cells divide once in about every three to four days including certain tumour cells. Although tumours contain rapidly dividing cells, some tumour cells especially those in the centre of the tumour, divide infrequently.

Alternatively the target cell may be a growth-arrested cell capable of undergoing cell division such as a cell in a central portion of a tumour mass or a stem cell such as a haematopoietic stem cell or a CD34-positive cell.

As a further alternative, the target cell may be a precursor of a differentiated cell such as a monocyte precursor, a CD33-positive cell, or a myeloid precursor.

As a further alternative, the target cell may be a differentiated cell such as a neuron, astrocyte, glial cell, microglial cell, macrophage, monocyte, epithelial cell, endothelial cell, hepatocyte, spermatocyte, spermatid or spermatozoa.

Target cells may be transduced either in vitro after isolation from a human individual or may be transduced directly in vivo.

Viral vectors according to the invention are retroviral vectors, in particular lentiviral vectors such as HIV and EIAV vectors. The retroviral vector of the present invention may be derived from or may be derivable from any suitable retrovirus. A large number of different retroviruses have been identified. Examples include: murine leukemia virus (MLV), human immunodeficiency virus (HIV), simian immunodeficiency virus, human T-cell leukemia virus (HTLV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV). A detailed list of retroviruses may be found in Coffin et al., 1997, “Retroviruses”, Cold Spring Harbour Laboratory Press Eds: J M Coffin, S M Hughes, H E Varmus pp 758-763.

The term “derivable” is used in its normal sense as meaning a nucleotide sequence such as an LTR or a part thereof which need not necessarily be obtained from a vector such as a retroviral vector but instead could be derived therefrom. By way of example, the sequence may be prepared synthetically or by use of recombinant DNA techniques.

Details on the genomic structure of some retroviruses may be found in the art. By way of example, details on HIV and Mo-MLV may be found from the NCBI Genbank (Genome Accession Nos. AF033819 and AF033811, respectively). Details of HIV variants may also be found at the http address hiv-web.lanl.gov. Details of EIAV variants may be found through at the http address www.ncbi.nlm.nih.gov.

The lentivirus group can be split even further into “primate” and “non-primate”. Examples of primate lentiviruses include human immunodeficiency virus (HIV), the causative agent of human auto-immunodeficiency syndrome (AIDS), and simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

The basic structure of a retrovirus genome is a 5′ LTR and a 3′ LTR, between or within which are located a packaging signal to enable the genome to be packaged, a primer binding site, integration sites to enable integration into a host cell genome and gag, pol and env genes encoding the packaging components, the latter of which are polypeptides required for the assembly of viral particles. More complex retroviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration, and transcription. LTRs also serve as enhancer-promoter sequences and can control the expression of the viral genes. Encapsidation of the retroviral RNAs occurs by virtue of a psi sequence, which it has been disclosed in respect of HIV, at least, is located at the 5′ end of the viral genome.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In a defective retroviral vector genome gag, pol and env may be absent or not functional. The R regions at both ends of the RNA are repeated sequences. U5 and U3 represent unique sequences at the 5′ and 3′ ends of the RNA genome respectively.

As discussed above, in a typical retroviral vector for use in gene therapy, at least part of one or more of the gag, pol and env protein coding regions essential for replication may be removed from the viral vector. This makes the retroviral vector replication-defective. The removed portions may even be replaced by a nucleotide sequence of interest (NOI), as in the present invention, to generate a virus capable of integrating its genome into a host genome but wherein the modified viral genome is unable to propagate itself due to a lack of structural proteins. When integrated in the host genome, expression of the NOI occurs, resulting in, for example, a therapeutic and/or a diagnostic effect. Thus, the transfer of an NOI into a site of interest is typically achieved by: integrating the NOI into the recombinant viral vector; packaging the modified viral vector into a virion coat; and allowing transduction of a site of interest—such as a targeted cell or a targeted cell population.

A minimal retroviral genome for use in the present invention may therefore comprise (5′) R—U5—one or more NOIs—U3-R (3′). However, the plasmid vector used to produce the retroviral genome within a host cell/packaging cell will also include transcriptional regulatory control sequences operably linked to the retroviral genome to direct transcription of the genome in a host cell/packaging cell. These regulatory sequences may be the natural sequences associated with the transcribed retroviral sequence, i.e. the 5′ U3 region, or they may be a heterologous promoter such as another viral promoter, for example the CMV promoter.

Some retroviral genomes require additional sequences for efficient virus production. For example, in the case of HIV, rev and RRE sequence should be included. However, it has been found that the requirement for rev and RRE can be reduced or eliminated by codon optimization. As expression of the codon optimized gag-pol is REV independent, RRE can be removed from the gag-pol expression cassette, thus removing any potential for recombination with any RRE contained on the vector genome.

Once the retroviral vector NOIs sequences need to be expressed. In a retrovirus, the promoter is located in the 5′ LTR U3 region of the provirus. In retroviral vectors, the promoter driving expression of a therapeutic gene may be the native retroviral promoter in the 5′ U3 region, or an alternative promoter engineered into the vector. The alternative promoter may physically replace the 5′ U3 promoter native to the retrovirus, or it may be incorporated at a different place within the vector genome such as between the LTRs.

Thus, the NOI will also be operably linked to a transcriptional regulatory control sequence to allow transcription of the first nucleotide sequence to occur in the target cell. The control sequence will typically be active in mammalian cells. The control sequence may, for example, be a viral promoter such as the natural viral promoter or a CMV promoter or it may be a mammalian promoter. A promoter sometimes is used that is preferentially active in a particular cell type or tissue type in which the virus to be treated primarily infects. Thus, in one embodiment, a tissue-specific regulatory sequences may be used. The regulatory control sequences driving expression of the one or more first nucleotide sequences may be constitutive or regulated promoters.

The term “operably linked” denotes a relationship between a regulatory region (typically a promoter element, but may include an enhancer element) and the coding region of a gene, whereby the transcription of the coding region is under the control of the regulatory region.

As used herein, the term “enhancer” includes a DNA sequence which binds other protein components of the transcription initiation complex and thus facilitates the initiation of transcription directed by its associated promoter. In one embodiment of the present invention, the enhancer is an ischaemic like response element (ILRE). The term “ischaemia like response element”-otherwise written as ILRE—includes an element that is responsive to or is active under conditions of ischaemia or conditions that are like ischaemia or are caused by ischaemia. By way of example, conditions that are like ischaemia or are caused by ischaemia include hypoxia and/or low glucose concentration(s). The term “hypoxia” refers to a condition under which a particular organ or tissue receives an inadequate supply of oxygen. Ischaemia can be an insufficient supply of blood to a specific organ or tissue. A consequence of decreased blood supply is an inadequate supply of oxygen to the organ or tissue (hypoxia). Prolonged hypoxia may result in injury to the affected organ or tissue. An ILRE sometimes utilized is a hypoxia response element (HRE).

In one aspect of the present invention, there is hypoxia or ischaemia regulatable expression of the retroviral vector components. In this regard, hypoxia is a powerful regulator of gene expression in a wide range of different cell types and acts by the induction of the activity of hypoxia-inducible transcription factors such as hypoxia inducible factor-1 (HIF-1; Wang & Semenza. 1993. proc Natl Acad. Sci. 90:430), which bind to cognate DNA recognition sites, the hypoxia-responsive elements (HREs) on various gene promoters. Dachs et al (Dachs et al. 1997. Nature Med. 5:515) have used a multimeric form of the HRE from the mouse phosphoglycerate kinase-1 (PGK-1) gene (Firth et al. 1994. proc Natl Acad. Sci. 90: 6496-6500) to control expression of both marker and therapeutic genes by human fibrosarcoma cells in response to hypoxia in vitro and within solid tumours in vivo (Dachs et al. 1997. Nature Med. 5:515).

Hypoxia response enhancer elements (HREEs) also have been found in association with a number of genes including the erythropoietin (EPO) gene (Madan et al. 1993. proc Natl Acad. Sci. 90:3928; Semenza & Wang. 1992. Mol Cell Biol. 1992. 12: 5447-5454). Other HREEs have been isolated from regulatory regions of both the muscle glycolytic enzyme pyruvate kinase (PKM) gene (Takenaka et al. 1989. J. Biol. Chem. 264: 2363-2367.), the human muscle-specific β-enolase gene (ENO3; Peshavaria & Day. 1991. Biochem J. 275: 427-433.) and the endothelin-1 (ET-1) gene (Inou et al. 1989. J. Biol. Chem. 264: 14954-14959).

The HRE of the present invention sometimes is selected from, for example, the erythropoietin HRE element (HREE1), muscle pyruvate kinase (PKM), HRE element, phosphoglycerate kinase (PGK) HRE, B-enolase (enolase 3; ENO3) HRE element, endothelin-1 (ET-1) HRE element and metallothionein 11 (MTII) HRE element. The ILRE sometimes is used in combination with a transcriptional regulatory element, such as a promoter, which transcriptional regulatory element often is active in one or more selected cell type(s), sometimes being only active in one cell type.

As outlined above, this combination aspect of the present invention is called a responsive element. The responsive element often comprises at least the ILRE as herein defined. Non-limiting examples of such a responsive element are presented as OBHRE1 and XiaMac. Another non-limiting example includes the ILRE in use in conjunction with an MLV promoter and/or a tissue restricted ischaemic responsive promoter. These responsive elements are disclosed in WO99/15684.

Other examples of suitable tissue restricted promoters/enhancers are those which are highly active in tumour cells such as a promoter/enhancer from a MUC1 gene, a CE4 gene or a 5T4 antigen gene. The alpha-fetoprotein (AFP) promoter is also a tumour-specific promoter. One promoter-enhancer combination sometimes utilized is a human cytomegalovirus (hCMV) major immediate early (MIE) promoter/enhancer combination. The term “promoter” is used in the normal sense of the art, e.g. an RNA polymerase binding site. The promoter may be located in the retroviral 5′ LTR to control the expression of a cDNA encoding an NOI, and/or gag-pol proteins. The NOI and/or gag-pol proteins often are capable of being expressed from the retrovirus genome such as from endogenous retroviral promoters in the long terminal repeat (LTR). The NOI and/or gag-pol proteins sometimes are expressed from a heterologous promoter to which the heterologous gene or sequence, and/or codon optimized gag-pol sequence is operably linked. Alternatively, the promoter may be an internal promoter. Often the NOI is expressed from an internal promoter.

Vectors containing internal promoters have also been widely used to express multiple genes. An internal promoter makes it possible to exploit promoter/enhancer combinations other than those found in the viral LTR for driving gene expression. Multiple internal promoters can be included in a retroviral vector and it has proved possible to express at least three different cDNAs each from its own promoter (Overell et al. 1988. Mol Cell Biol. 8: 1803-1808). Internal ribosomal entry site (IRES) elements have also been used to allow translation of multiple coding regions from either a single mRNA or from fusion proteins that can then be expressed from an open reading frame.

The promoter of the present invention may be constitutively efficient, or may be tissue or temporally restricted in their activity. The promoter often is a constitutive promoter such as CMV. Also, the promoters of the present invention often are tissue specific. That is, they are capable of driving transcription of a NOI or NOI(s) in one tissue while remaining largely “silent” in other tissue types. The term “tissue specific” refers to a promoter which is not restricted in activity to a single tissue type but which nevertheless shows selectivity in that they may be active in one group of tissues and less active or silent in another group.

The level of expression of an NOI or NOIs under the control of a particular promoter may be modulated by manipulating the promoter region. For example, different domains within a promoter region may possess different gene regulatory activities. The roles of these different regions are typically assessed using vector constructs having different variants of the promoter with specific regions deleted (that is, deletion analysis). This approach may be used to identify, for example, the smallest region capable of conferring tissue specificity or the smallest region conferring hypoxia sensitivity.

A number of tissue specific promoters, described above, may be particularly advantageous in practising the present invention. In most instances, these promoters may be isolated as convenient restriction digestion fragments suitable for cloning in a selected vector. Alternatively, promoter fragments may be isolated using the polymerase chain reaction. Cloning of the amplified fragments may be facilitated by incorporating restriction sites at the 5′ end of the primers.

The NOI or NOIs may be under the expression control of an expression regulatory element, such as a promoter and enhancer. Often the ischaemic responsive promoter is a tissue restricted ischaemic responsive promoter. Also the tissue restricted ischaemic responsive promoter sometimes is a macrophage specific promoter restricted by repression. Sometimes the tissue restricted ischaemic responsive promoter is an endothelium specific promoter. The regulated retroviral vector often is an ILRE regulated retroviral vector. Sometimes the regulated retroviral vector is an ILRE regulated lentiviral vector. The regulated retroviral vector often is an autoregulated hypoxia responsive lentiviral vector.

Also, the regulated retroviral vector of the present invention sometimes is regulated by glucose concentration. For example, the glucose-regulated proteins (grp's) such as grp78 and grp94 are highly conserved proteins known to be induced by glucose deprivation (Attenello & Lee. 1984. Science. 226: 187-190). The grp 78 gene is expressed at low levels in most normal healthy tissues under the influence of basal level promoter elements but has at least two critical “stress inducible regulatory elements” upstream of the TATA element (Attenello & Lee. 1984. Science. 226: 187-190; Gazit et al. 1985. Cancer Res. 55: 1660-1663). Attachment to a truncated 632 base pair sequence of the 5′end of the grp78 promoter confers high inducibility to glucose deprivation on reporter genes in vitro (Gazit et al. 1985. Cancer Res. 55: 1660-1663). Furthermore, this promoter sequence in retroviral vectors was capable of driving a high level expression of a reporter gene in tumour cells in murine fibrosarcomas, particularly in central relatively ischaemic/fibrotic sites (Gazit et al. 1985. Cancer Res. 55: 1660-1663).

Often, the regulated retroviral vector of the present invention is a self-inactivating (SIN) vector. By way of example, self-inactivating retroviral vectors have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive provirus (Yu et al. 1986. proc Natl Acad. Sci. 83: 3194-3198; Dougherty & Temin. 1987. proc Natl Acad. Sci. 84: 1197-1201; Hawley et al. 1987. proc Natl Acad. Sci. 84: 2406-2410; Yee, J. K., A. Miyanohara, P. LaPorte, K. Bouic, J. C. Burns, and T. Friedmann. 1994. Proc. Natl. Acad. Sci. USA. 91:9564-8). However, any promoter(s) internal to the LTRs in such vectors will still be transcriptionally active. This strategy has been employed to eliminate effects of the enhancers and promoters in the viral LTRs on transcription from internally placed genes. Such effects include increased transcription (Jolley et al. 1983. Nucleic Acids Res. 11: 1855-1872) or suppression of transcription (Emerman & Tenim. 1984. Cell. 39: 449-467). This strategy can also be used to eliminate downstream transcription from the 3′ LTR into genomic DNA (Herman & Coffin. 1987. Science. 236: 845-848). This is of particular concern in human gene therapy where it is of critical importance to prevent the adventitious activation of an endogenous oncogene.

As discussed above, replication-defective retroviral vectors are typically propagated, for example to prepare suitable titres of the retroviral vector for subsequent transduction, by using a combination of a packaging or helper cell line and the recombinant vector. That is to say, that the three packaging proteins can be provided in trans.

In general a “packaging cell line” contains one or more of the retroviral gag, pol and env genes. In the present invention it contains codon optimized gag-pol genes, and optionally an env gene. The packaging cell line produces the proteins required for packaging retroviral DNA but it cannot bring about encapsidation. Conventionally this has been achieved through lack of a psi region. However, when a recombinant vector carrying an NOI and apsi region is introduced into the packaging cell line, the helper proteins can package the psi-positive recombinant vector to produce the recombinant virus stock. This virus stock can be used to transduce cells to introduce the NOI into the genome of the target cells. Conventionally a psi packaging signal, called psi plus, has been used that contains additional sequences spanning from upstream of the splice donor to downstream of the gag start codon (Bender et al., 1987, J Virol 61: 1639-1646) since this has been shown to increase viral titres.

The recombinant virus whose genome lacks all genes required to make viral proteins can tranduce only once and cannot propagate. These viral vectors which are only capable of a single round of transduction of target cells are known as replication defective vectors. Hence, the NOI is introduced into the host/target cell genome without the generation of potentially harmful retrovirus. A summary of the available packaging lines is presented in Coffin et al., 1997 (ibid).

The retroviral packaging cell line sometimes is in the form of a transiently transfected cell line. Transient transfections may advantageously be used to measure levels of vector production when vectors are being developed. In this regard, transient transfection avoids the longer time required to generate stable vector-producing cell lines and may also be used if the vector or retroviral packaging components are toxic to cells. Components typically used to generate retroviral vectors include a plasmid encoding the gag-pol proteins, a plasmid encoding the env protein and a plasmid containing an NOI. Vector production involves transient transfection of one or more of these components into cells containing the other required components. If the vector encodes tokic genes or genes that interfere with the replication of the host cell, such as inhibitors of the cell cycle or genes that induce apotosis, it may be difficult to generate stable vector-producing cell lines, but transient transfection can be used to produce the vector before the cells die. Also, cell lines have been developed using transient transfection that produce vector titre levels that are comparable to the levels obtained from stable vector-producing cell lines (Pear et al., 1993, Proc Natl Acad Sci 90: 8392-8396).

Producer cells/packaging cells can be of any suitable cell type. Producer cells are generally mammalian cells but can be, for example, insect cells. A producer cell may be a packaging cell containing the virus structural genes, normally integrated into its genome into which the regulated retroviral vectors of the present invention are introduced. Alternatively the producer cell may be transfected with nucleic acid sequences encoding structural components, such as codon optimized gag-pol and env on one or more vectors such as plasmids, adenovirus vectors, herpes viral vectors or any method known to deliver functional DNA into target cells. The vectors according to the present invention are then introduced into the packaging cell by the methods of the present invention.

As used herein, the term “producer cell” or “vector producing cell” refers to a cell which contains all the elements necessary for production of regulated retroviral vector particles and regulated retroviral delivery systems. Often, the producer cell is obtainable from a stable producer cell line, and the producer cell is sometimes obtainable from a derived stable producer cell line. Also, the producer cell may be obtainable from a derived producer cell line. As used herein, the term “derived producer cell line” is a transduced producer cell line which has been screened and selected for high expression of a marker gene. Such cell lines contain retroviral insertions in integration sites that support high level expression from the retroviral genome. The term “derived producer cell line” is used interchangeably with the term “derived stable producer cell line” and the term “stable producer cell line. Often, the derived producer cell line includes but is not limited to a retroviral and/or a lentiviral producer cell. Also, the derived producer cell line sometimes is an HIV or EIAV producer cell line, and more frequently an EIAV producer cell line.

The envelope protein sequences, and nucleocapsid sequences often are all stably integrated in the producer and/or packaging cell. However, one or more of these sequences could also exist in episomal form and gene expression could occur from the episome.

As used herein, the term “packaging cell” refers to a cell which contains those elements necessary for production of infectious recombinant virus which are lacking in a recombinant viral vector. Typically, such packaging cells contain one or more vectors which are capable of expressing viral structural proteins (such as codon optimized gag-pol and env) but they do not contain a packaging signal.

The term “packaging signal” which is referred to interchangeably as “packaging sequence” or “psi” is used in reference to the non-coding, cis-acting sequence required for encapsidation of retroviral RNA strands during viral particle formation. In HIV-1, this sequence has been mapped to loci extending from upstream of the major splice donor site (SD) to at least the gag start codon.

Packaging cell lines suitable for use with the above-described vector constructs may be readily prepared (see also WO92/05266), and utilized to create producer cell lines for the production of retroviral vector particles. As already mentioned, a summary of the available packaging lines is presented in “Retroviruses” (1997 Cold Spring Harbour Laboratory Press Eds: J M Coffin, S M Hughes, H E Varmus pp 449).

Also as discussed above, simple packaging cell lines, comprising a provirus in which the packaging signal has been deleted, have been found to lead to the rapid production of undesirable replication competent viruses through recombination. In order to improve safety, second generation cell lines have been produced wherein the 3′LTR of the provirus is deleted. In such cells, two recombinations would be necessary to produce a wild type virus. A further improvement involves the introduction of the gag-pol genes and the env gene on separate constructs so-called third generation packaging cell lines. These constructs are introduced sequentially to prevent recombination during transfection (Danos & Mulligan. 1998. proc Natl Acad. Sci. 85: 6460-6464; Markowitz et al. 1988. Virology. 167: 400-406).

The packaging cell lines often are second generation packaging cell lines, and sometimes the packaging cell lines are third generation packaging cell lines. In these split-construct, third generation cell lines, a further reduction in recombination may be achieved by “codon wobbling”. This technique, based on the redundancy of the genetic code, aims to reduce homology between the separate constructs, for example between the regions of overlap in the gag-pol and env open reading frames.

The packaging cell lines are useful for providing the gene products necessary to encapsidate and provide a membrane protein for a high titre regulated retrovirus vector and regulated nucleic gene delivery vehicle production. When regulated retrovirus sequences are introduced into the packaging cell lines, such sequences are encapsidated with the nucleocapsid (gag-pol) proteins and these units then bud through the cell membrane to become surrounded in cell membrane and to contain the envelope protein produced in the packaging cell line. These infectious regulated retroviruses are useful as infectious units per se or as gene delivery vectors.

The packaging cell may be a cell cultured in vitro such as a tissue culture cell line. Suitable cell lines include but are not limited to mammalian cells such as murine fibroblast derived cell lines or human cell lines. The packaging cell line sometimes is a human cell line, such as for example: HEK293, 293-T, TE671, HT1080. Alternatively, the packaging cell may be a cell derived from the individual to be treated such as a monocyte, macrophage, blood cell or fibroblast. The cell may be isolated from an individual and the packaging and vector components administered ex vivo followed by re-administration of the autologous packaging cells.

High-titre virus preparations can be used in both experimental and practical applications. Techniques for increasing viral titre include using a psi plus packaging signal as discussed above and concentration of viral stocks. In addition, the use of different envelope proteins, such as the G protein from vesicular-stomatitis virus has improved titres following concentration to 109 per ml (Cosset et al., 1995, J. Virol. 69: 7430-7436). However, typically the envelope protein will be chosen such that the viral particle will preferentially infect cells that are infected with the virus which it desired to treat. For example where an HIV vector is being used to treat HIV infection, the env protein used will be the HIV env protein. As used herein, the term “high titre” refers to an effective amount of a retroviral vector or particle which is capable of transducing a target site such as a cell. The titre often is at least 10⁶ retrovirus particles per ml, such as from 10⁶ to 10⁷ per ml, sometimes at least 10⁷ retrovirus particles per ml. As used herein, the term “effective amount” refers to an amount of a regulated retroviral or lentiviral vector or vector particle which is sufficient to induce expression of an NOI at a target site.

The process of producing a retroviral vector in which the envelope protein is not the native envelope of the retrovirus is known as “pseudotyping”. Certain envelope proteins, such as MLV envelope protein and vesicular stomatitis virus G (VSV-G) protein, pseudotype retroviruses very well. Pseudotyping is not a new phenomenon and examples may be found in WO-A-98/05759, WO-A-98/05754, WO-A-97/17457, WO-A-96/09400, WO-A-91/00047 and (Mebatsion et al. 1997. Cell. 90: 841-847).

It is possible to manipulate the viral genome or the regulated retroviral vector nucleotide sequence, so that viral genes are replaced or supplemented with one or more NOIs which may be heterologous NOIs. The term “heterologous” refers to a nucleic acid sequence or protein sequence linked to a nucleic acid or protein sequence which it is not naturally linked. The term NOI (i.e. nucleotide sequence of interest) includes any suitable nucleotide sequence, which need not necessarily be a complete naturally occurring DNA sequence. Thus, the DNA sequence can be, for example, a synthetic DNA sequence, a recombinant DNA sequence (i.e. prepared by use of recombinant DNA techniques), a cDNA sequence or a partial genomic DNA sequence, including combinations thereof. The DNA sequence need not be a coding region. If it is a coding region, it need not be an entire coding region. In addition, the DNA sequence can be in a sense orientation or in an anti-sense orientation. Often it is in a sense orientation and the DNA often is or comprises cDNA. The NOI(s) may be any one or more of selection gene(s), marker gene(s) and therapeutic gene(s).

As used herein, the term “selection gene” refers to the use of a NOI which encodes a selectable marker which may have an enzymatic activity that confers resistance to an antibiotic or drug upon the cell in which the selectable marker is expressed.

Many different selectable markers have been used successfully in retroviral vectors. These are reviewed in “Retroviruses” (1997 Cold Spring Harbour Laboratory Press Eds: J M Coffin, S M Hughes, H E Varmus pp 444) and include, but are not limited to, the bacterial neomycin (neo) and hygromycin phosphotransferase genes which confer resistance to G418 and hygromycin respectively; a mutant mouse dihydrofolate reductase gene which confers resistance to methotrexate; the bacterial gpt gene which allows cells to grow in medium containing mycophenolic acid, xanthine and aminopterin; the bacterial hisD gene which allows cells to grow in medium without histidine but containing histidinol; the multidrug resistance gene (mdr) which confers resistance to a variety of drugs; and the bacterial genes which confer resistance to puromycin or phleomycin. All of these markers are dominant selectable and allow chemical selection of most cells expressing these genes. Other selectable markers are not dominant in that their use must be in conjunction with a cell line that lacks the relevant enzyme activity. Examples of non-dominant selectable markers include the thymidine kinase (tk) gene which is used in conjunction with tk cell lines.

Markers that can be utilized herein are blasticidin and neomycin, optionally operably linked to a thymidine kinase coding sequence typically under the transcriptional control of a strong viral promoter such the SV40 promoter.

In accordance with the present invention, suitable NOI sequences include those that are of therapeutic and/or diagnostic application such as, but are not limited to: sequences encoding cytokines, chemokines, hormones, antibodies, engineered immunoglobulin-like molecules, a single chain antibody, fusion proteins, enzymes, immune co-stimulatory molecules, immunomodulatory molecules, anti-sense RNA, a transdominant negative mutant of a target protein, a toxin, a conditional toxin, an antigen, a tumour suppressor protein and growth factors, membrane proteins, vasoactive proteins and peptides, anti-viral proteins and ribozymes, and derivatives thereof (such as with an associated reporter group). When included, such coding sequences may be typically operatively linked to a suitable promoter, which may be a promoter driving expression of a ribozyme(s), or a different promoter or promoters, such as in one or more specific cell types.

Suitable NOIs for use in the invention in the treatment or prophylaxis of cancer include NOIs encoding proteins which: destroy the target cell (for example a ribosomal toxin), act as: tumour suppressors (such as wild-type p53); activators of anti-tumour immune mechanisms (such as cytokines, co-stimulatory molecules and immunoglobulins); inhibitors of angiogenesis; or which provide enhanced drug sensitivity (such as pro-drug activation enzymes); indirectly stimulate destruction of target cell by natural effector cells (for example, strong antigen to stimulate the immune system or convert a precursor substance to a toxic substance which destroys the target cell (for example a prodrug activating enzyme).

Examples of prodrugs include but are not limited to etoposide phosphate (used with alkaline phosphatase; 5-fluorocytosine (with cytosine deaminase); Doxorubin-N-p-hydroxyphenoxyacetamide (with Penicillin-V-Amidase); Para-N-bis (2-chloroethyl)aminobenzoyl glutamate (with Carboxypeptidase G2); Cephalosporin nitrogen mustard carbamates (with B-lactamase); SR4233 (with p450 reductase); Ganciclovir (with HSV thymidine kinase); mustard pro-drugs with nitroreductase and cyclophosphamide or ifosfamide (with cytochrome p450).

Suitable NOIs for use in the treatment or prevention of ischaemic heart disease include NOIs encoding plasminogen activators. Suitable NOIs for the treatment or prevention of rheumatoid arthritis or cerebral malaria include genes encoding anti-inflammatory proteins, antibodies directed against tumour necrosis factor (TNF) alpha, and anti-adhesion molecules (such as antibody molecules or receptors specific for adhesion molecules).

The expression products encoded by the NOIs may be proteins which are secreted from the cell. Alternatively the NOI expression products are not secreted and are active within the cell. In either event, the NOI expression product often demonstrates a bystander effect or a distant bystander effect; that is the production of the expression product in one cell leading to the killing of additional, related cells, either neighbouring or distant (e.g. metastatic), which possess a common phenotype. Encoded proteins could also destroy bystander tumour cells (for example with secreted antitumour antibody-ribosomal toxin fusion protein), indirectly stimulated destruction of bystander tumour cells (for example cytokines to stimulate the immune system or procoagulant proteins causing local vascular occlusion) or convert a precursor substance to a toxic substance which destroys bystander tumour cells (eg an enzyme which activates a prodrug to a diffusible drug). Also, the delivery of NOI(s) encoding antisense transcripts or ribozymes which interfere with expression of cellular genes for tumour persistence (for example against aberrant myc transcripts in Burkitts lymphoma or against bcr-abl transcripts in chronic myeloid leukemia. The use of combinations of such NOIs is also envisaged.

The NOI or NOIs of the present invention may also comprise one or more cytokine-encoding NOIs. Suitable cytokines and growth factors include but are not limited to: ApoE, Apo-SAA, BDNF, Cardiotrophin-1, EGF, ENA-78, Eotaxin, Eotaxin-2, Exodus-2, FGF-acidic, FGF-basic, fibroblast growth factor-10 (Marshall. 1998. Nature Biotechnology. 16: 129). FLT3 ligand, Fractalkine (CX3C), GDNF, G-CSF, GM-CSF, GF-β1, insulin, IFN-γ, IGF-I, IGF-II, IL-1α, IL-1β, IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein (Marshall. 1998. Nature Biotechnology. 16: 129), M-CSF, MDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, α-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β, TGF-β2, TGF-β3, tumour necrosis factor (TNF), TNF-α, TNF-β, TNIL-1, TPO, VEGF, GCP-2, GRO/MGSA, GRO-β, GRO-γ, HCC1, 1-309.

The NOI or NOIs may be under the expression control of an expression regulatory element, such as a promoter and/or a promoter enhancer as known as “responsive elements” in the present invention.

When the regulated retroviral vector particles are used to transfer NOIs into cells which they transduce, such vector particles also designated “viral delivery systems” or “retroviral delivery systems”. Viral vectors, including retroviral vectors, have been used to transfer NOIs efficiently by exploiting the viral transduction process. NOIs cloned into the retroviral genome can be delivered efficiently to cells susceptible to transduction by a retrovirus. Through other genetic manipulations, the replicative capacity of the retroviral genome can be destroyed. The vectors introduce new genetic material into a cell but are unable to replicate.

The regulated retroviral vector of the present invention can be delivered by viral or non-viral techniques. Non-viral delivery systems include but are not limited to DNA transfection methods. Here, transfection includes a process using a non-viral vector to deliver a gene to a target mammalian cell.

Typical transfection methods include electroporation, DNA biolistics, lipid-mediated transfection, compacted DNA-mediated transfection, liposomes, immunoliposomes, lipofectin, cationic agent-mediated, cationic facial amphiphiles (CFAs) (Nature Biotechnology. 1996. 14:556), multivalent cations such as spermine, cationic lipids or polylysine, 1,2,-bis (oleoyloxy)-3-(trimethylammonio) propane (DOTAP)-cholesterol complexes (Wolff& Trubetskoy. 1998. Nature Biotechnology. 16: 421) and combinations thereof.

Viral delivery systems include but are not limited to adenovirus vector, an adeno-associated viral (AAV) vector, a herpes viral vector, a retroviral vector, a lentiviral vector, or a baculoviral vector. These viral delivery systems may be configured as a split-intron vector. A split intron vector is described in WO99/15683.

Other examples of vectors include ex vivo delivery systems, which include but are not limited to DNA transfection methods such as electroporation, DNA biolistics, lipid-mediated transfection, compacted DNA-mediated transfection.

The vector may be a plasmid DNA vector. Alternatively, the vector may be a recombinant viral vector. Suitable recombinant viral vectors include adenovirus vectors, adeno-associated viral (AAV) vectors, Herpes-virus vectors, or retroviral vectors, lentiviral vectors or a combination of adenoviral and lentiviral vectors. In the case of viral vectors, gene delivery is mediated by viral infection of a target cell.

If the features of adenoviruses are combined with the genetic stability of retro/lentiviruses then essentially the adenovirus can be used to transduce target cells to become transient retroviral producer cells that could stably infect neighbouring cells.

The present invention also provides a pharmaceutical composition for treating an individual by gene therapy, wherein the composition comprises a therapeutically effective amount of a regulated retroviral vector according to the present invention. The pharmaceutical composition may be for human or animal usage. Typically, a physician will determine the actual dosage which will be most suitable for an individual subject and it will vary with the age, weight and response of the particular patient.

The composition may optionally comprise a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilizing agent(s), and other carrier agents that may aid or increase the viral entry into the target site (such as for example a lipid delivery system).

Where appropriate, the pharmaceutical compositions can be administered by any one or more of: minipumps, inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intracavemosally, intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

The present invention is believed to have a wide therapeutic applicability—depending on inter alia the selection of the one or more NOIs.

For example, the present invention may be useful in the treatment of the disorders listed in WO-A-98/05635. For ease of reference, part of that list is now provided: cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the present invention may be useful in the treatment of disorders listed in WO-A-98/07859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilizing specific cell types to sites of injury or infection); haemostatic and thrombolytic activity (e.g. for treating haemophilia and stroke); antiinflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the present invention may be useful in the treatment of disorders listed in WO-A-98/09985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

Boilerplate for the Examples

### EXAMPLE 1

### HIV

Cell Lines

T cells (DuBridge, R. B., P. Tang, H. C. Hsia, P.-M. Leong, J. H. Miller, and M. P. Calos. 1987. Mol. Cell. Biol. 7:379-387) and HeLa cells (Gey, G. O., W. D. Coffman, and M. T. Kubicek. 1952. Cancer res. 12:264) were maintained in Dubecco's modified Eagle's medium containing 10% (v/v) fetal calf serum and supplemented with L-glutamine and antibiotics (penicillin-streptomycin). 293T cells were obtained from D. Baltimore (Rockefeller University).

HIV-1 Proviral Clones

Proviral clones pWI3 (Kim, S. Y., R. Bym, J. Groopman, and D. Baltimore. 1989. J. Virol. 63:3708-3713) and pNL4-3 (Adachi, A., H. Gendelman, S. Koenig, T. Folks, R. Willey, A. Rabson, and M. Martin. 1986. J. Virol. 59:284-291) were used.

Construction of a Packaging System

In one of the present examples, a modified codon optimized HIV env sequence is used (SEQ ID NO:4). The corresponding env expression plasmid is designated pSYNgp160nm. The modified sequence contains extra motifs not used by (Haas, J., E.-C. Park, and B. Seed. 1996. Current Biology. 6:315). The extra sequences were taken from the HIV env sequence of strain MN and codon optimized. Any similar modification of the nucleic acid sequence would function similarly as long as it used codons corresponding to abundant tRNAs (Zolotukhin, S., M. Potter, W. W. Hauswirth, J. Guy, and N. Muzyczka. 1996. A “humanized” green fluorescent protein cDNA adapted for high-level expression in mammalian cells. J. Virol. 70:4646-54).

Codon Optimized HIV-1 Gag-Pol Gene

A codon optimized gag-pol gene, shown from nt 1108 to 5414 of SEQ ID NO:2 was constructed by annealing a series of short overlapping oligonucleotides (approximately 30-40 mers with 25% overlap, i.e. approximately 9 nucleotides). Oligonucleotides were purchased from R&D SYSTEMS (R&D Systems Europe Ltd, 4-10 The Quadrant, Barton Lane, Abingdon, OX14 3YS, UK). Codon optimization was performed using the sequence of HXB-2 strain (AC: K03455) (Fisher, A., E. Collalti, L. Ratner, R. Gallo, and F. Wong-Staal.

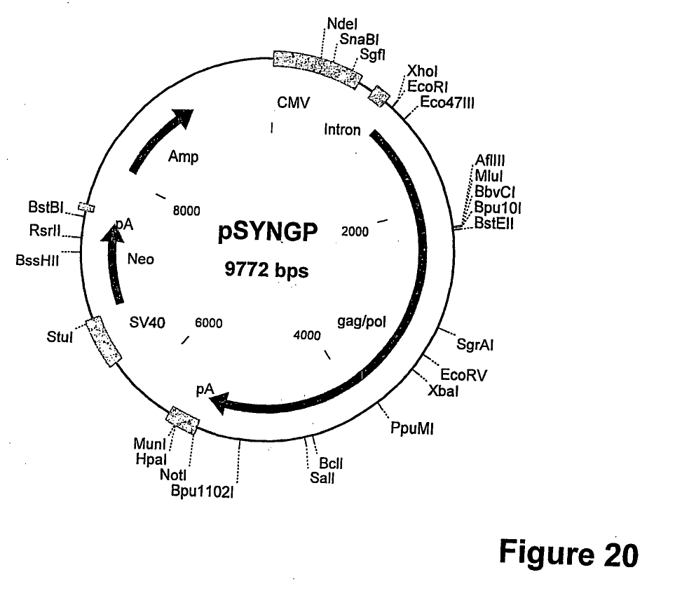

1985. Nature. 316:262-265). The Kozak consensus sequence for optimal translation initiation (Kozak, M. 1992. [Review]. Annu. Rev. Cell Biol. 8:197-225) was also included. A fragment from base 1222 from the beginning of gag until the end of gag (1503) was not optimized in order to maintain the frameshift site and the overlap between the gag and pol reading frames. This was from clone pNL4-3. (When referring to base numbers within the gag-pol gene base 1 is the A of the gag ATG, which corresponds to base 790 from the beginning of the HXB2 sequence. When referring to sequences outside the gag-pol then the numbers refer to bases from the beginning of the HXB2 sequence, where base 1 corresponds to the beginning of the 5′ LTR). Some deviations from optimization were made in order to introduce convenient restriction sites. The final codon usage is shown in FIG. 3b, which now resembles that of highly expressed human genes and is quite different from that of the wild type HIV-1 gag-pol. The gene was cloned into the mammalian expression vector pCIneo (Promega) in the EcoRI-NotI sites. The resulting plasmid was named pSYNGP (FIG. 20, SEQ ID NO:2). Sequencing of the gene in both strands verified the absence of any mistakes. A sequence comparison between the codon optimized and wild type HIV gag-pol sequence is shown in FIG. 8.

Rev/RRE Constructs

The HIV-1 RRE sequence (bases 7769-8021 of the HXB2 sequence) was amplified by PCR from pWI3 proviral clone with primers bearing the NotI restriction site and was subsequently cloned into the NotI site of pSYNGP. The resulting plasmids were named pSYNGP-RRE (RRE in the correct orientation) and pSYNGP-ERR(RRE in the reverse orientation).

Pseudotyped Viral Particles

In one form of the packaging system a synthetic gag-pol cassette is coexpressed with a heterologous envelope coding sequence. This could be for example VSV-G (Ory, D. S., B. A. Neugeboren, and R. C. Mulligan. 1996. proc Natl Acad Sci USA. 93:11400-6, Zhu, Z. H., S. S. Chen, and A. S. Huang. 1990. J Acquir Immune Defic Syndr. 3:215-9), amphotropic MLV env (Chesebro, B., K. Wehrly, and W. Maury. 1990. J. Virol. 64:4553-7, Spector, D. H., E. Wade, D. A. Wright, V. Koval, C. Clark, D. Jaquish, and S. A. Spector. 1990. J. Virol. 64:2298-2308) or any other protein that would be incorporated into the HIV or EIAV particle (Valsesia Wittmann, S., A. Drynda, G. Deleage, M. Aumailley, J. M. Heard, O. Danos, G. Verdier, and F. L. Cosset. 1994. J. Virol. 68:4609-19). This includes molecules capable of targeting the vector to specific tissues.

HIV-1 Vector Genome Constructs
pH6nZ is derived from pH4Z (Kim, V. N., K. Mitrophanous, S. M. Kingsman, and K. A. J. 1998. J Virol 72: 811-816) by the addition of a single nucleotide to place an extra guanine residue that was missing from pH4Z at the 5=end of the vector genome transcript to optimize reverse transcription. In addition the gene coding for β-galactosidase (LacZ) was replaced by a gene encoding for a nuclear localising β-galactosidase. (Martin-Rendon and Said Ismail provided pH6nZ). In order to construct Rev(−) genome constructs the following modifications were made: a) A 1.8 kb PstI—PstI fragment was removed from pH6nZ, resulting in plasmid pH6.1nZ and b) an EcoNI (filled)—SphI fragment was substituted with a SpeI (filled)—SphI fragment from the same plasmid (pH6nZ), resulting in plasmid pH6.2nZ. In both cases sequences within gag (nt 1-625) were retained, as they have been shown to play a role in packaging (93). Rev, RRE and any other residual env sequences were removed. pH6.2nZ further contains the env splice acceptor, whereas pH6.1nZ does not.

A series of vectors encompassing further gag deletions plus or minus a mutant major splice donor (SD) (GT to CA mutation) were also derived from pH6Z. These were made by PCR with primers bearing a NarI (5=primers) and an SpeI (3=primers) site. The PCR products were inserted into pH6Z at the NarI-SpeI sites. The resulting vectors were named pHS1nZ (containing HIV-1 sequences up to gag 40), pHS2nZ (containing HIV-1 sequences up to gag 260), pHS3nZ (containing HIV-1 sequences up to gag 360), pHS4nZ (containing HIV-1 sequences up to gag 625), pHS5nZ (same as pHS1nZ but with a mutant SD), pHS6nZ (same as pHS2nZ but with a mutant SD), pHS7nZ (same as pHS3nZ but with a mutant SD) and pHS8nZ (same as pHS4nZ but with a mutant SD).

In addition, the RRE sequence (nt 7769-8021 of the HXB2 sequence) was inserted in the SpeI (filled) site of pH6.1nZ, pHS1nZ, pHS3nZ and pHS7nZ resulting in plasmids pH6.1nZR, pHS1nZR, pHS3nZR and pHS7nZR respectively.

Other modifications to the genome have been made including the generation of a SIN vector (by deletion of part of the 3=U3), the replacement of the LTRs with those from MLV or replacement of part of the 3=U3 with the MLV U3 region.

Transient Transfections, Transductions and Determination of Viral Titres

These were performed as previously described (Kim, V. N., K. Mitrophanous, S. M. Kingsman, and K. A. J. 1998. J Virol 72: 811-816, Soneoka, Y., P. M. Cannon, E. E. Ramsdale, J. C. Griffiths, G. Romano, S. M. Kingsman, and A. J. Kingsman. 1995. Nucleic Acids Res. 23:628-33). Briefly, 293T cells were seeded on 6 cm dishes and 24 hours later they were transiently transfected by overnight calcium phosphate treatment. The medium was replaced 12 hours post-transfection and unless otherwise stated supernatants were harvested 48 hours post-transfection, filtered (through 0.22 or 0.45 μm filters) and titered by transduction of 293T cells. For this reason supernatant at appropriate dilutions of the original stock was added to 293T cells (plated onto 6 or 12 well plates 24 hours prior to transduction). 8 μg/ml Polybrene (Sigma) was added to each well and 48 hours post transduction viral titres were determined by X-gal staining.

Luminescent β-galactosidase (β-gal) Assays

These were performed on total cell extracts using a luminescent β-gal reporter system (CLONTECH). Untransfected 293T cells were used as negative control and 293T cells transfected with pCMV-β-gal (CLONTECH) were used as positive control.

RNA Analysis

Total or cytoplasmic RNA was extracted from 293T cells by using the RNeasy mini kit (QUIAGEN) 36-48 hours post-transfection. 5-10 μg of RNA was subjected to Northern blot analysis as previously described (Sagerstrom, C., and H. Sive. 1996. RNA blot analysis, p. 83-104. In P. Krieg (ed.), A laboratory guide to RNA: isolation, analysis and synthesis, vol. 1. Wiley-Liss Inc., New York). Correct fractionation was verified by staining of the agarose gel. A probe complementary to bases 1222-1503 of the gag-pol gene was amplified by PCR from HIV-1 pNL4-3 proviral clone and was used to detect both the codon optimized and wild type gag-pol mRNAs. A second probe, complementary to nt 1510-2290 of the codon optimized gene was also amplified by PCR from plasmid pSYNGP and was used to detect the codon optimized genes only. A 732 bp fragment complementary to all vector genomes used in this study was prepared by an SpeI-AvrII digestion of pH6nZ. A probe specific for ubiquitin (CLONTECH) was used to normalise the results. All probes were labelled by random labelling (STRATAGENE) with α-³²P dCTP (Amersham). The results were quantitated by using a Storm Phosphorlmager (Molecular Dynamics) and shown in FIG. 12. In the total cellular fractions the 47S rRNA precursor could be clearly seen, whereas it was absent from the cytoplasmic fractions. As expected (Malim, M. H., J. Hauber, S. Y. Le, J. V. Maizel, and B. R. Cullen. 1989. Nature. 338:254-7), Rev stimulates the cytoplasmic accumulation of wild type gag-pol mRNA (lanes 1c and 2c). RNA levels were 10-20 fold higher for the codon optimized gene compared to the wild type one, both in total and cytoplasmic fractions (compare lanes 3t-2t, 3c-2c, 10c-8c). The RRE sequence did not significantly destabilise the codon optimized RNAs since RNA levels were similar for codon optimized RNAs whether or not they contained the RRE sequence (compare lanes 3 and 5). Rev did not markedly enhance cytoplasmic accumulation of the codon optimized gag-pol mRNAs, even when they contained the RRE sequence (differences in RNA levels were less than 2-fold, compare lanes 3-4 or 5-6).

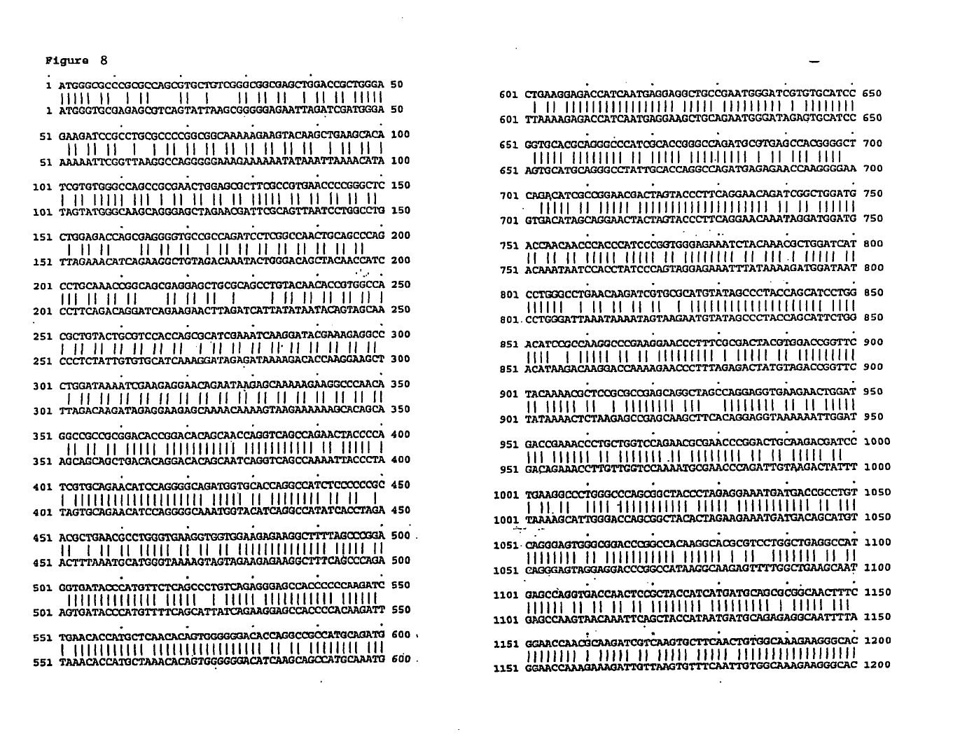

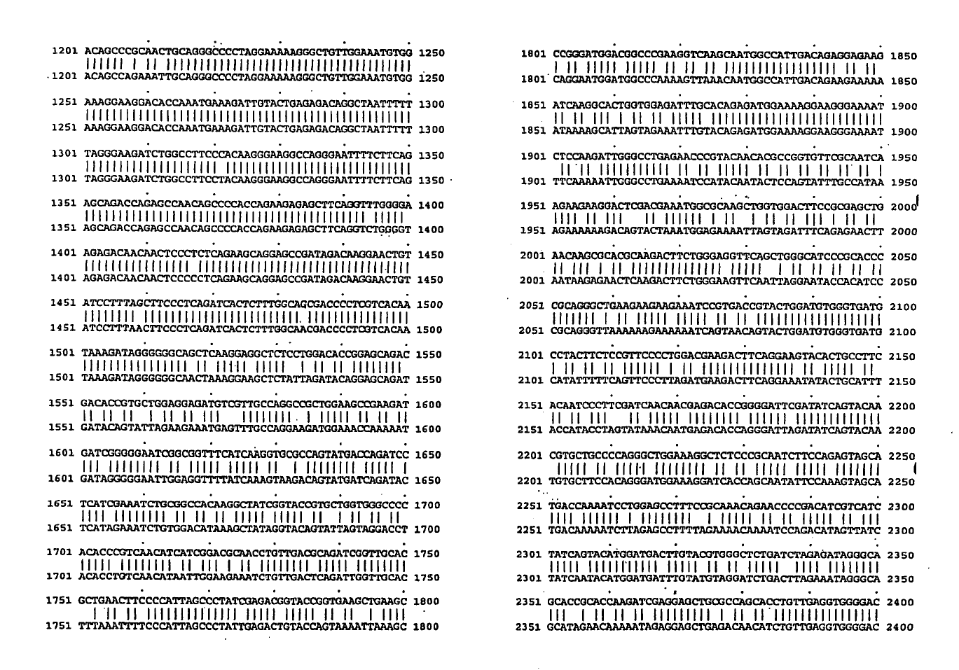

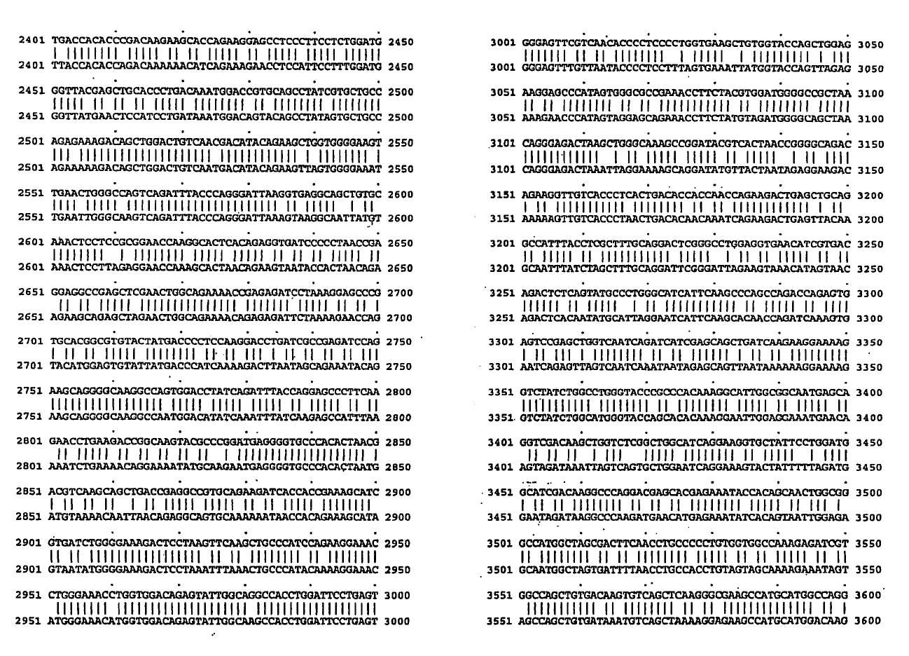

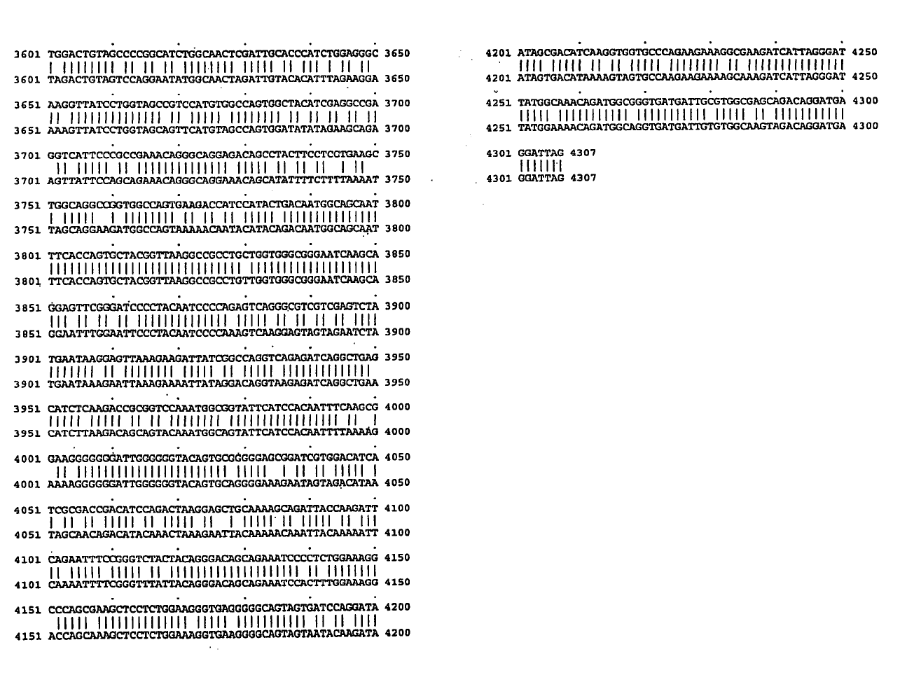

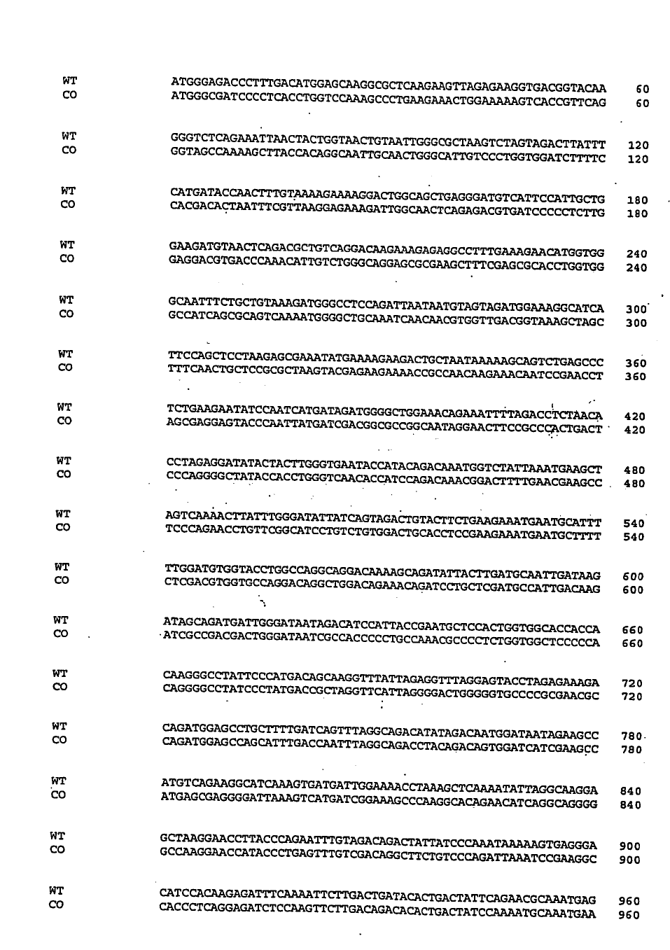

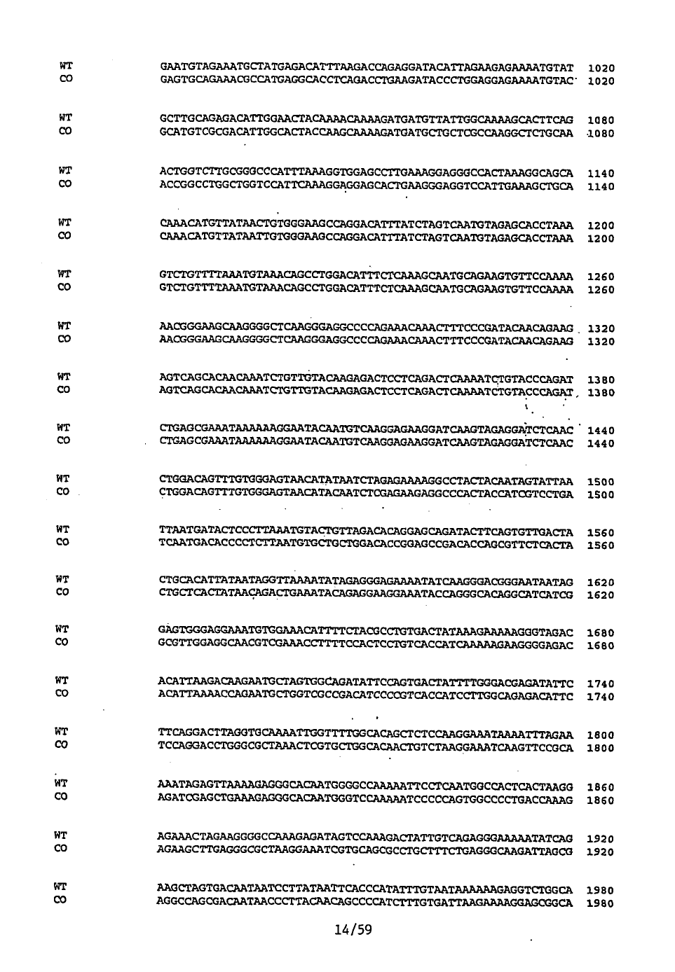

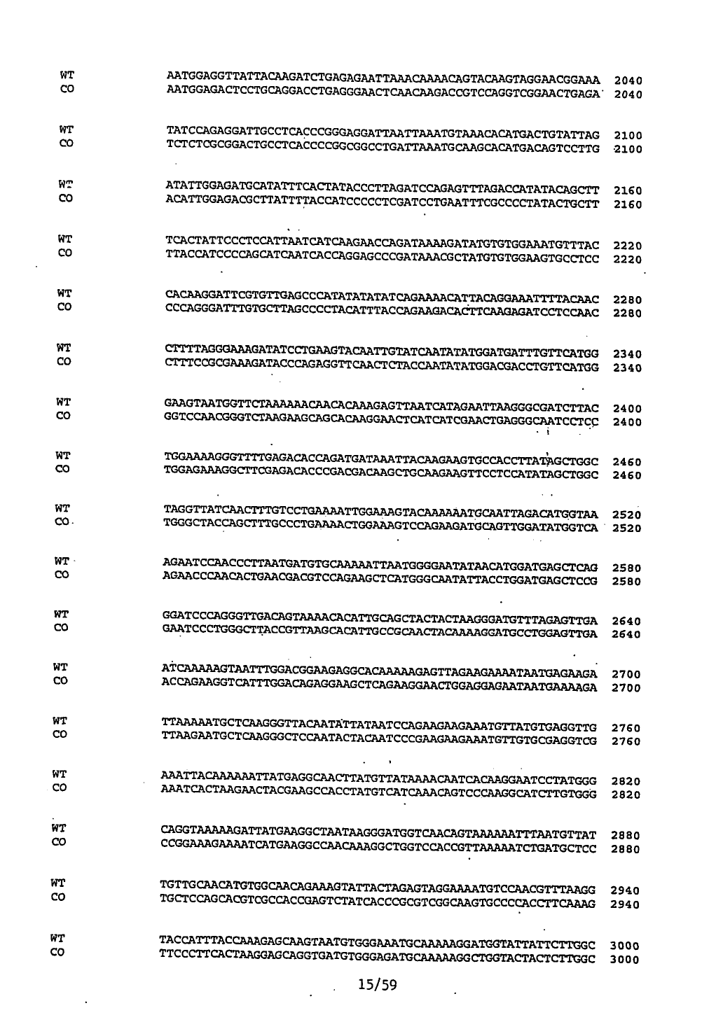

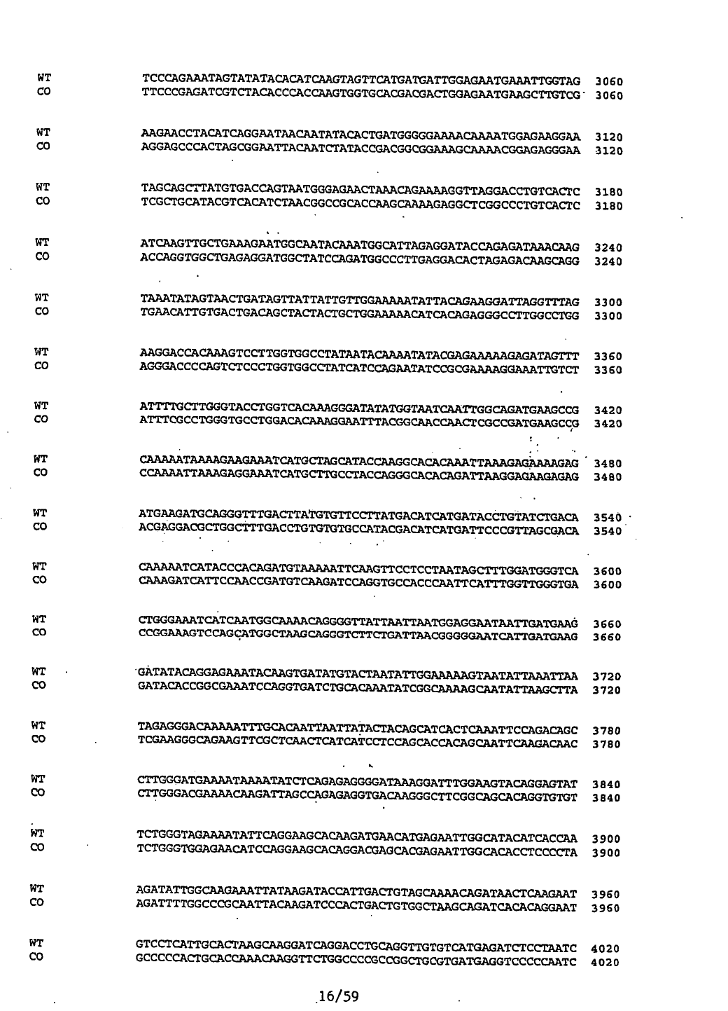

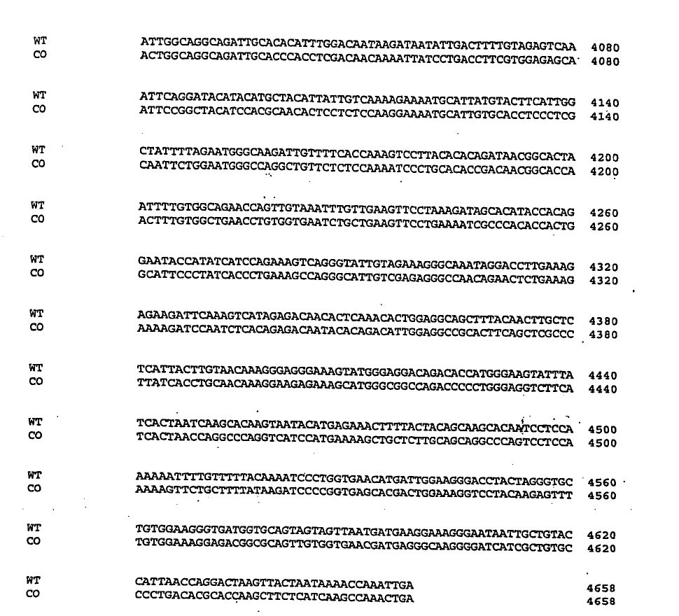

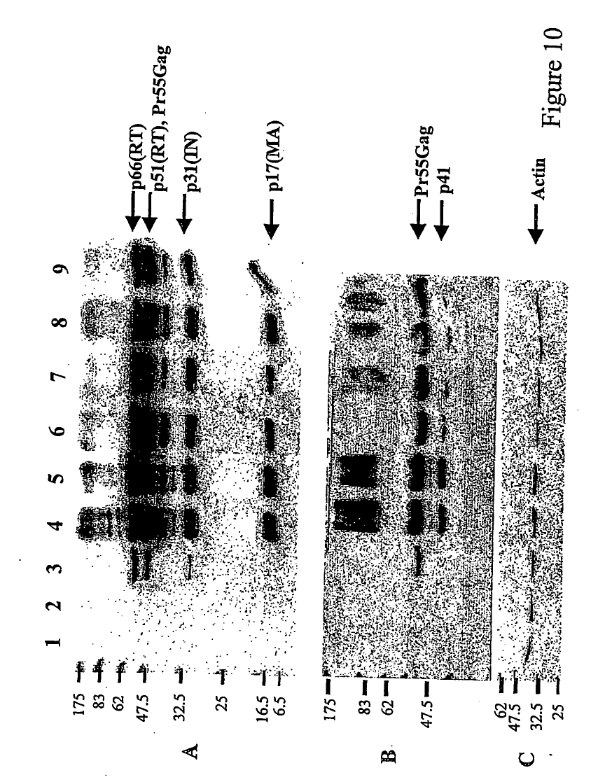

It appeared from a comparison of FIGS. 10 and 12 that all of the increase in protein expression from syngp could be accounted for by the increase in RNA levels. To investigate whether this was due to saturating levels of RNA in the cell, 0.1, 1 and 10 μg of the wild type or codon optimized expression vectors were transfected into 293T cells and compared protein production. In all cases protein production was 10-fold higher for the codon optimized gene for the same amount of transfected DNA, while increase in protein levels was proportional to the amount of transfected DNA for each individual gene. It seems likely therefore that the enhanced expression of the codon optimized gene can be mainly attributed to the enhanced RNA levels present in the cytoplasm and not to increased translation.

Protein Analysis

Total cell lysates were prepared from 293T cells 48 hours post-transfection (unless otherwise stated) with an alkaline lysis buffer. For extraction of proteins from cell supernatants the supernatant was first passed through a 0.22 μm filter and the vector particles were collected by centrifugation of 1 ml of supernatant at 21,000 g for 30 minutes. Pellets were washed with PBS and then re-suspended in a small volume (2-10 il) of lysis buffer. Equal protein amounts were separated on a SDS 10-12% (v/v) polyacrylamide gel. Proteins were transferred to nitrocellulose membranes which were probed sequentially with a 1:500 dilution of HIV-1 positive human serum (AIDS Reagent Project, ADP508, Panel E) and a 1:1000 dilution of horseradish peroxidase labelled anti-human IgG (Sigma, A0176). Proteins were visualised using the ECL or ECL-plus western blotting detection reagent (Amersham). To verify equal protein loading, membranes were stripped and re-probed with a 1:1000 dilution of anti-actin antibody (Sigma, A2066), followed by a 1:2000 dilution of horseradish peroxidase labelled anti-rabbit IgG (Vector Laboratories, PI-1000).

Expression of Gag-Pol Gene Products and Vector Particle Production

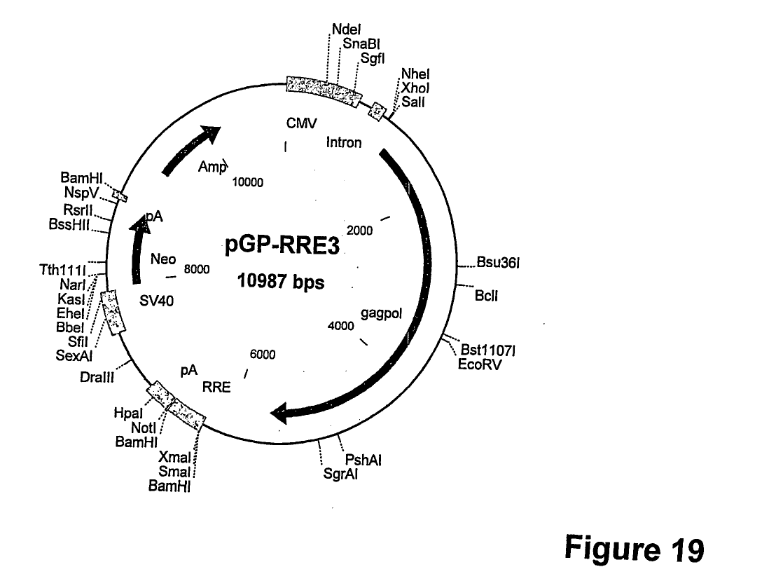

The wild type gag-pol (pGP-RRE3 B FIG. 19) (Kim, V. N., K. Mitrophanous, S. M. Kingsman, and K. A. J. 1998. J Virol 72: 811-816), and codon optimized expression vectors (PSYNGP, pSYNGP-RRE and pSYNGP-ERR) were transiently transfected into 293T cells. Transfections were performed in the presence or absence of a Rev expression vector, pCMV-Rev (Felber, B. K., M. Hadzopoulou Cladaras, C. Cladaras, T. Copeland, and G. N. Pavlakis. 1989. Proc. Natl. Acad. Sci. USA. 86:1495-1499), in order to assess Rev-dependence for expression. Western blot analysis was performed on cell lysates and supernatants to assess protein production. The results are shown in FIG. 10. As expected (Hadzopoulou Cladaras, M., B. K. Felber, C. Cladaras, A. Athanassopoulos, A. Tse, and G. N. Pavlakis. 1989. J. Virol. 63:1265-74), expression of the wild type gene is observed only when Rev is provided in trans (lanes 2 and 3). In contrast, when the codon optimized gag-pol was used, there was high level expression in both the presence and absence of Rev (lanes 4 and 5), indicating that in this system there was no requirement for Rev. Protein levels were higher for the codon optimized gene than for the wild type gag-pol (compare lanes 4-9 with lane 3). The difference was more evident in the cell supernatants (approximately 10-fold higher protein levels for the codon optimized gene compared to the wild type one, quantitated by using a Phosphorlmager) than in the cell lysates.

In previous studies where the RRE has been included in gag-pol expression vectors that had been engineered to remove INS sequences, inclusion of the RRE lead to a decrease in protein levels, that was restored by providing Rev in trans (Schneider, R., M. Campbell, G. Nasioulas, B. K. Felber, and G. N. Pavlakis. 1997. J. Virol. 71: 4892-903). In our hands, the presence of the RRE in the fully codon optimized gag-pol mRNA did not affect protein levels and provision of Rev in trans did not further enhance expression (lanes 6 and 7).

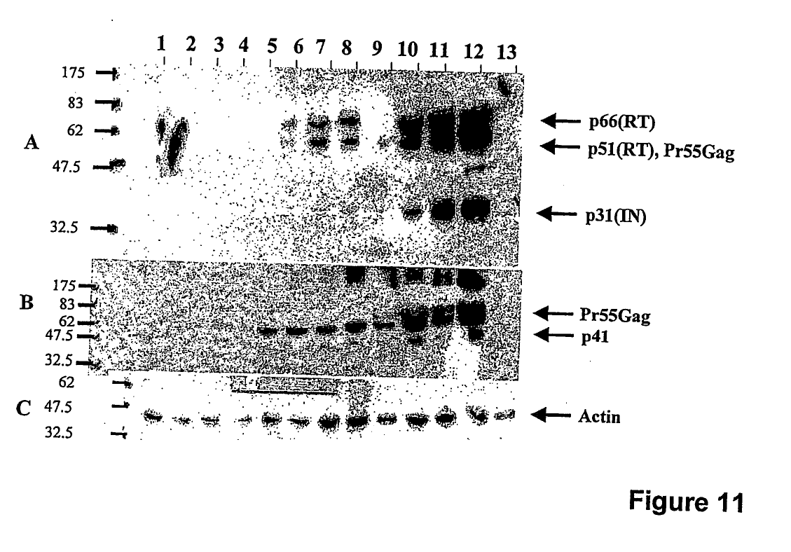

In order to compare translation rates between the wild type and codon optimized gene, protein production from the wild type and codon optimized expression vector was determined at several time intervals post transfection into 293T cells. Protein production and particle formation was determined by Western blot analysis and the results are shown in FIG. 11. Protein production and particle formation was 10-fold higher for the codon optimized gag-pol at all time points.

To further determine whether this enhanced expression that was observed with the codon optimized gene was due to better translation or due to effects on the RNA, RNA analysis was carried out.

The Efficiency of Vector Production Using the Codon Optimized Gag-Pol Gene

To determine the effects of the codon optimized gag-pol on vector production, the HIV vector genome pH6nZ and the VSV-G envelope expression plasmid pHCMVG (113) were used in combination with either pSYNGP, pSYNGP-RRE, pSYNGP-ERR or pGP-RRE3 as a source for the gag-pol in a plasmid ratio of 2:1:2 in a 3 plasmid co-transfection of 293T cells (Kim, V. N., K. Mitrophanous, S. M. Kingsman, and K. A. J. 1998. J Virol 72: 811-816). Whole cell extracts and culture supernatants were evaluated by Western blot analysis for the presence of the gag and gag-pol gene products. Particle production was, as expected (FIG. 10), 5-10 fold higher for the codon optimized genes when compared to the wild type.

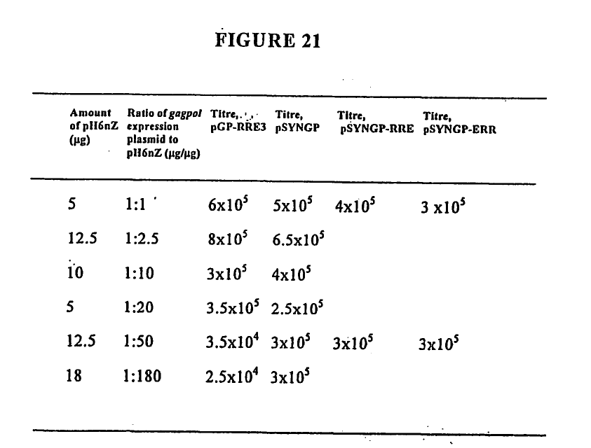

To determine the effects of the codon optimized gag-pol gene on vector titres, several ratios of the vector components were used. The results are shown in FIG. 21. Where the gag-pol was the limiting component in the system (as determined by the drop in titres observed with the wild type gene), titres were 10-fold higher for the codon optimized vectors. This is in agreement with the higher protein production observed for these vectors, but suggests that under normal conditions of vector production gag-pol is saturating and the codon optimization gives no maximum yield advantage.

The Effect of HIV-1 Gag INS Sequences on the Codon Optimized Gene is Position Dependent

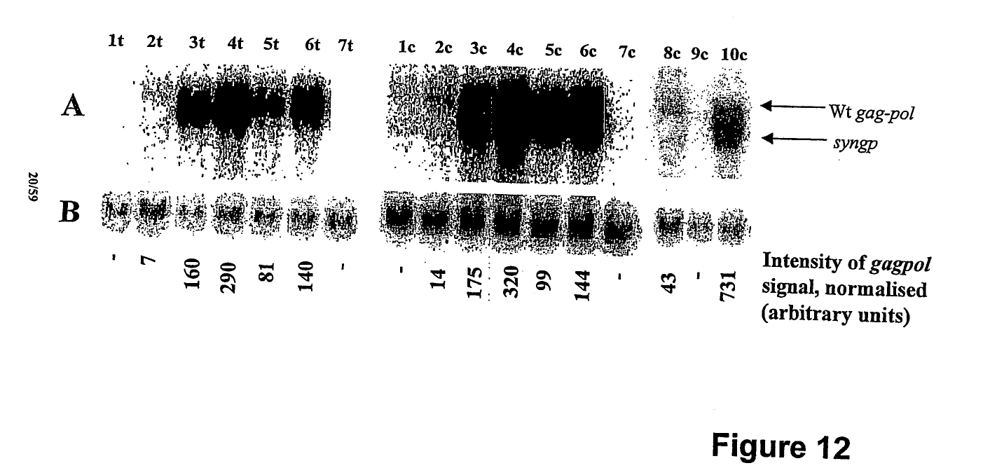

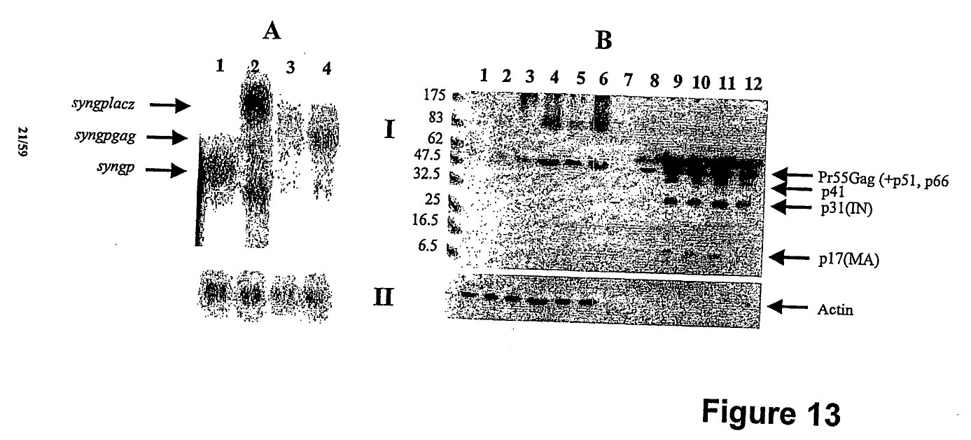

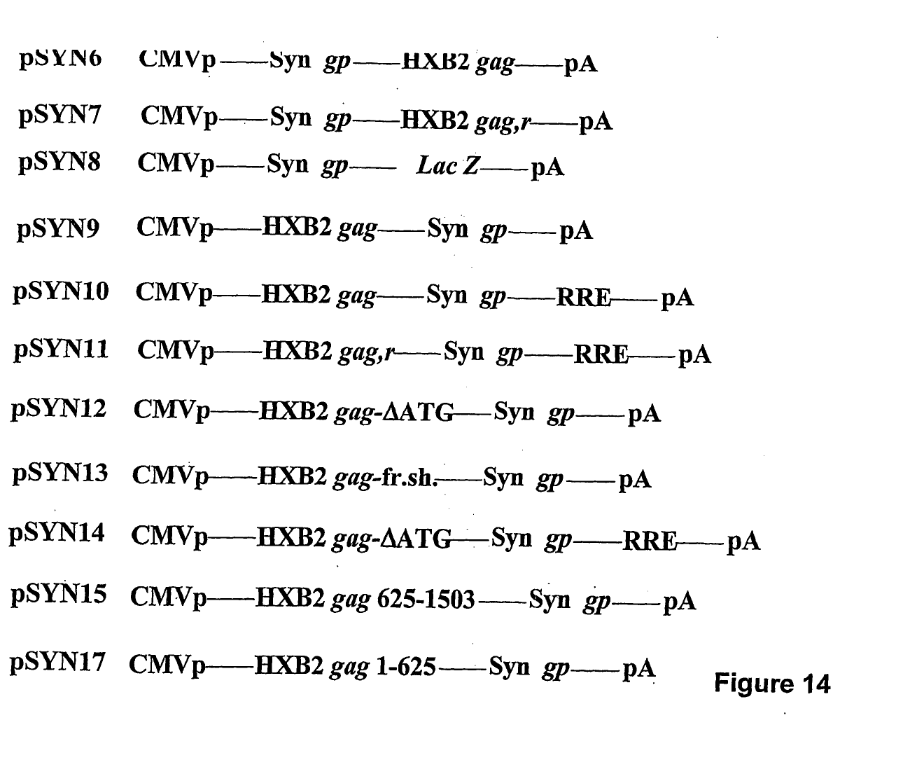

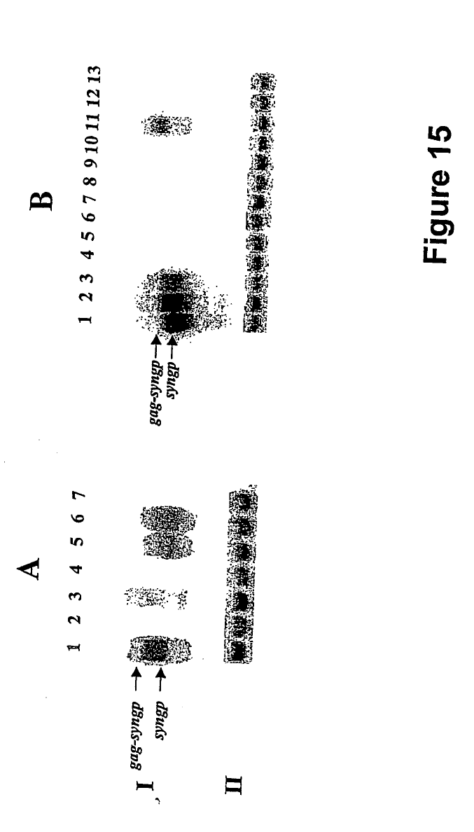

It has previously been demonstrated that insertion of wild type HIV-1 gag sequences downstream of other RNAs, e.g. HIV-1 tat (Schwartz, S., B. K. Felber, and G. N. Pavlakis. 1992. J. Virol. 66:150-159), HIV-1 gag (Schneider, R., M. Campbell, G. Nasioulas, B. K. Felber, and G. N. Pavlakis. 1997. J. Virol. 71: 4892-903) or CAT (Maldarelli, F., M. A. Martin, and K. Strebel. 1991 J. Virol. 65:5732-5743) can lead to a dramatic decrease in steady state mRNA levels, presumably as a result of the INS sequences. In other cases, e.g. for α-globin (Mikaelian, I., M. Krieg, M. Gait, and J. Kam. 1996. J. Mol. Biol. 257:246-264), it was shown that the effect was splice site dependent. Cellular AREs (AU-rich elements) that are found in the 3=UTR of labile mRNAs may confer mRNA destabilisation by inducing cytoplasmic deadenylation of the transcripts (Xu, N., C.-Y. Chen, and A.-B. Shyu. 1997. Mol. Cell. Biol. 17:4611-4621). To test whether HIV-1 gag INS sequences would destabilise the codon optimized RNA, the wild-type HIV-1 gag sequence, or parts of it (nt 1-625 or nt 625-1503), were amplified by PCR from the proviral clone pW13. All fragments were blunt ended and were inserted into pSYNGP or pSYNGP-RRE at either a blunted EcoRI or NotI site (upstream or downstream of the codon optimized gag-pol gene repectively). As controls the wt HIV-1 gag in the reverse orientation (as INS sequences have been shown to act in an orientation dependent manner, (Maldarelli, F., M. A. Martin, and K. Strebel. 1991 J. Virol. 65:5732-5743) (pSYN7) and lacZ, excised from plasmid pCMV-βgal (CLONTECH) (in the correct orientation) (pSYN8) were also inserted in the same site. Contrary to our expectation, as shown in FIG. 13, the wild type HIV-1 gag sequence did not appear to significantly affect RNA or protein levels of the codon optimized gene. Another series of plasmids was further constructed by PCR and from the same plasmids where the wild type HIV-1 gag in the sense or reverse orientation, subfragments of gag (nt 1-625 or nt 625-1503), the wild type HIV-1 gag without the ATG or with a frameshift mutation 25 bases downstream of the ATG, or nt 72-1093 of LacZ (excised from plasmid pH6Z), or the first 1093 bases of lacZ with or without the ATG were inserted upstream of the codon optimized HIV-1 gag-pol gene in pSYNGP and/or pSYNGP-RRE (pSYN9-pSYN22, FIG. 14). Northern blot analysis showed that insertion of the wild type HIV-1 gag gene upstream of the codon optimized HIV-1 gag-pol (pSYN9, pSYN10) lead to diminished RNA levels in the presence or absence of Rev/RRE (FIG. 15A, lanes 1-4 and FIG. 15B, lanes 1+12). The effect was not dependent on translation as insertion of a wild type HIV-1 gag lacking the ATG or with a frameshift mutation (pSYN12, pSYN13 and pSYN14) also diminished RNA levels (FIG. 15B, lanes 1-7). Western blot analysis verified that there was no HIV-1 gag translation product for pSYN12-14. However, it is possible that, as the wt HIV-1 gag exhibits such an adverse codon usage, it may act as a non-translatable long 5=leader for syngp, and if this is the case, then the ATG mutation should not have any effects.

Insertion of smallerpartsofthewild type HIV-1 gaggene (pSYN15 and pSYN17) also lead to a decrease in RNA levels (FIG. 15B, lanes 1-3 and 8-9), but not to levels as low as when the whole gag sequence was used (lanes 1-3, 4-7 and 8-9 in FIG. 15B). This indicates that the effect of INS sequences is dependent on their size. Insertion of the wild type HIV-1 gag in the reverse orientation (PSYN11) had no effect on RNA levels (FIG. 15A, lanes 1 and 5-6). However a splicing event seemed to take place in that case, as indicated by the size of the RNA (equal to the size of the codon optimized gag-pol RNA) and by the translation product (gag-pol, in equal amounts compared to pSYNGP, as verified by Western blot analysis).

These data indicate therefore that wild type HIV-1 gag instability sequences act in a position and size dependent manner, probably irrespective of translation. It should also be noted that the RRE was unable to rescue the destabilised RNAs through interaction with Rev.

Construction of an HIV-1 Based Vector System That Lacks All the Accessory Proteins

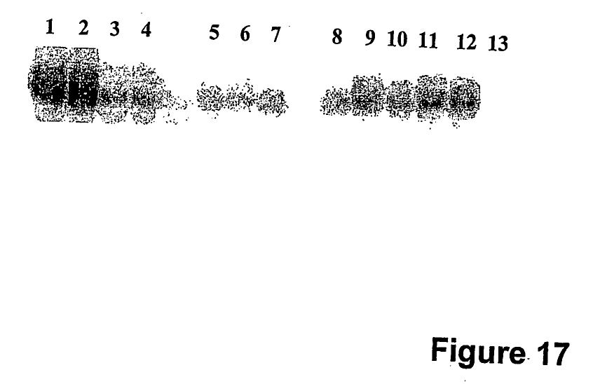

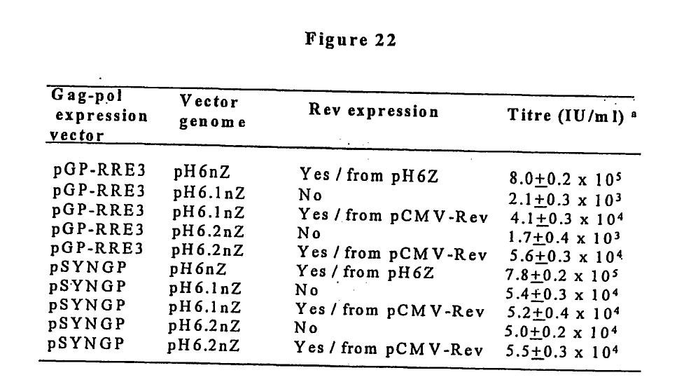

Until now several HIV-1 based vector systems have been reported that lack all accessory proteins but Rev (Kim, V. N., K. Mitrophanous, S. M. Kingsman, and K. A. J. 1998. J Virol 72: 811-816, Naldini, L. 1998. Curr. Opin. Biotechnol. 9:457-463). To investigate whether the codon optimized gene would permit the construction of an HIV-1 based vector system that lacks all accessory proteins, rev/RRE and any residual env sequences were initially deleted, but the first 625 nucleotides of gag were kept, as they have been shown to play a role in efficient packaging (Parolin, C., T. Dorfman, G. Palu, H. Gottlinger, and J. Sodroski. 1994J. Virol). Two vector genome constructs were made, pH6.1nZ (retaining only HIV sequences up to nt 625 of gag) and pH6.2nZ (same as pH6.1nZ, but also retaining the env splice acceptor). These were derived from a conventional HIV vector genome that contains RRE and expresses Rev (pH6nZ). Our 3-plasmid vector system now expressed only HIV-1 gag-pol and the VSV-G envelope proteins. Vector particle titres were determined as described in the previous section. A ratio of 2:2:1 of vector genome (pH6Z or pH6.1nZ or pH6.2nZ): gag-pol expression vector (pGP-RRE3 or pSYNGP): pHCMV-G was used. Transfections were performed in the presence or absence of pCMV-Rev, as gag-pol expression was still Rev dependent for the wild type gene. The results are summarised in FIG. 22 and indicate that an HIV vector could be produced in the total absence of Rev, but that maximum titres were compromised at 20-fold lower than could be achieved in the presence of Rev. As gag-pol expression should be the same for pSYNGP with pH6nZ or pH6.1nZ or pH6.2nZ (since it is Rev independent), as well as for pGP-RRE3 when Rev is provided in trans, it was suspected that the vector genome retained a requirement for Rev and was therefore limiting the titres. To confirm this, Northern blot analysis was performed on cytoplasmic RNA prepared from cells transfected with pH6nZ or pH6.1nZ in the presence or absence of pCMV-Rev. As can be seen in FIG. 17, lanes 1-4, the levels of cytoplasmic RNA derived from pH6nZ were 5-10 fold higher than those obtained with pH6.1nZ (compare lanes 1-2 to lanes 3-4). These data support the notion that RNA produced from the vector genome requires the Rev/RRE system to ensure high cytoplasmic levels. This may be due to inefficient nuclear export of the RNA, as INS sequences residing within gag were still present.

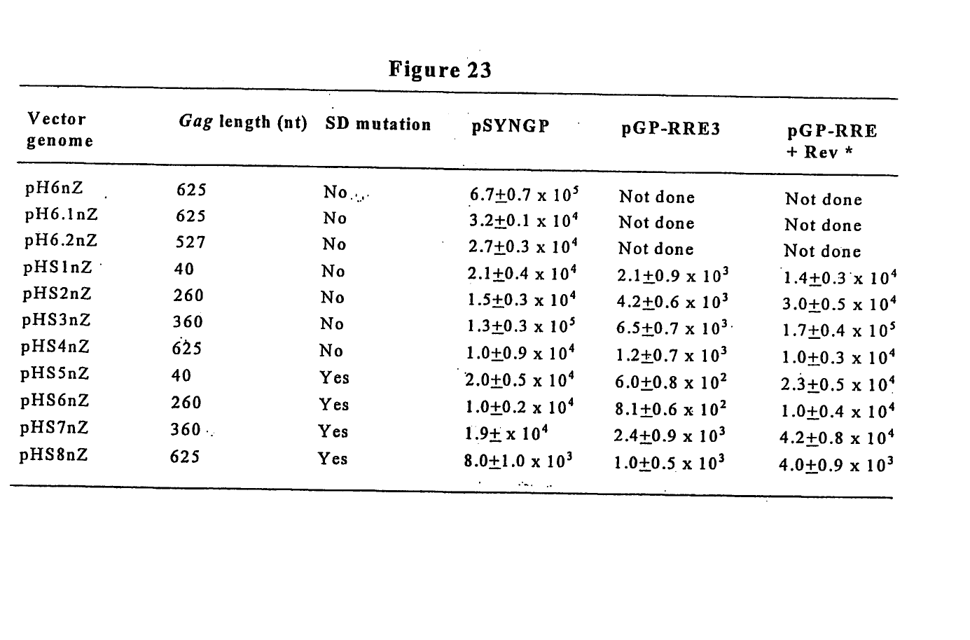

Further deletions in the gag sequences of the vector genome might therefore be necessary to restore titres. To date efficient packaging has been reported to require 360 (Dull, T., R. Zufferey, M. Kelly, R. Mandel, M. Nguyen, D. Trono, and L. Naldini. 1998. J. Virol. 72:8463-8471) or 255 (Cui, Y., T. Iwakama, and L.-J. Chang. 1999. J. Virol. 73:6171-6176) nucleotides of gag in vectors that still retain env sequences, or about 40 nucleotides of gag in a particular combination of splice donor mutation, gag and env deletions (Chang, L.-J., V. Urlacher, T. Iwakama, Y. Cui, and J. Zucali. 1999. Gene Ther. 6:715-728, Cui, Y., T. Iwakama, and L.-J. Chang. 1999. J. Virol. 73:6171-6176). In an attempt to remove the requirement for Rev/RRE in our vector genome without compromising efficient packaging, a series of vectors derived from pH6nZ containing progressively larger deletions of HIV-1 sequences (only sequences upstream and within gag were retained) plus and minus a mutant major splice donor (SD) (GT to CA mutation) was constructed. Vector particle titres were determined as before and the results are summarised in FIG. 23. As can be seen, deletion of up to nt 360 in gag (vector pHS3nZ) resulted in an increase in titres (compared to pH6.1nZ or pH6.2nZ) and only a 5-fold decrease (titres were 1.3-1.7×10⁵) compared to pH6nZ. Further deletions resulted in titres lower than pHS3nZ and similar to pH6.1nZ. In addition, the SD mutation did not have a positive effect on vector titres and in the case of pHS3nZ it resulted in a 10-fold decrease in titres (compare titres for pHS3nZ and pHS7nZ in FIG. 23). Northern blot analysis on cytoplasmic RNA (FIG. 17, lanes 1 and 5-12) showed that RNA levels were indeed higher for pH6nZ, which could account for the maximum titres observed with this vector. RNA levels were equal for pHS1nZ (lane 5), pHS2nZ (lane 6) and pHS3nZ (lane 7) whereas titres were 5-8 fold higher for pHS3nZ. It is possible that further deletions (than that found in pHS3nZ) in gag might result in less efficient packaging (as for HIV-1 the packaging signal extends in gag) and therefore even though all 3 vectors produce similar amounts of RNA only pHS3nZ retains maximum packaging efficiency. It is also interesting to note that the SD mutation resulted in increased RNA levels in the cytoplasm (compare lanes 6 and 10, 7 and 11 or 8 and 12 in FIG. 17) but equal or decreased titres (FIG. 23). The GT dinucleotide that was mutated is in the stem of SL2 of the packaging signal (Harrison, G., G. Miele, E. Hunter, and A. Lever. 1998. J. Virol. 72:5886-5896). It has been reported that SL2 might not be very important for HIV-1 RNA encapsidation (Harrison, G., G. Miele, E. Hunter, and A. Lever. 1998. J. Virol. 72:5886-5896, McBride, M. S., and A. T. Panganiban. 1997. J. Virol. 71:2050-8), whereas SL3 is of great importance (Lever, A., H. Gottlinger, W. Haseltine, and J. Sodroski. 1989 J. Virol. 63:4085-7). Folding of the wild type and SD-mutant vector sequences with the RNAdraw software program revealed that the mutation alters significantly the secondary structure of the RNA and not only of SL2. It is likely therefore that although the SD mutation enhances cytoplasmic RNA levels it does not increase titres as it alters the secondary structure of the packaging signal.

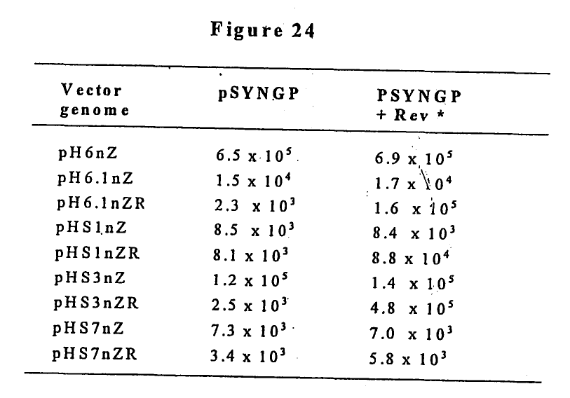

To investigate whether the titre differences that were observed with the Rev minus vectors were indeed due to Rev dependence of the genomes, the RRE sequence (nt 7769-8021 of the HXB2 sequence) was inserted in the SpeI site (downstream of the gag sequence and just upstream of the internal CMV promoter) of pH6.1nZ, pHS1nZ, pHS3nZ and pHS7nZ, resulting in plasmids pH6.1nZR, pHS1nZR, pHS3nZR and pHS7nZR respectively. Vector particle titres were determined with pSYNGP and pHCMVG in the presence or absence of Rev (pCMV-Rev) as before and the results are summarised in FIG. 24. In the absence of Rev titres were further compromised for pH6.1nZR (7-fold compared to pH6.1nZ), pHS3nZR (6-fold compared to pHS3nZ) and pHS7nZR (2.5-fold compared to pHS7nZ). This was expected, as the RRE also acts as an instability sequence (Brighty, D., and M. Rosenberg. 1994. proc. Natl. Acad. Sci. USA. 91:8314-8318) and so it would be expected to confer Rev-dependence. In the presence of Rev titres were restored to the maximum titres observed for pH6nZ in the case of pHS3nZR (5×10⁵) and pH6.1nZR (2×10⁵). Titres were not restored for pHS7nZR in the presence of Rev. This supports the hypothesis that the SD mutation in pHS7nZ affects the structure of the packaging signal and thus the packaging ability of this vector genome, as in this case Rev may be able to stimulate vector genome RNA levels, as for pHS3nZR and pH6.1nZR, but it can not affect the secondary structure of the packaging signal. For vector pHS1nZ inclusion of the RRE did not lead to a decrease in titres. This could be due to the fact that pHS1 nZ contains only 40 nucleotides of gag sequences and therefore even with the RRE the size of instability sequences is not higher than for pHS2nZ that gives equal titres to pHS1nZ. Rev was able to partially restore titres for pHS1nZR (10-fold increase when compared to pHS1nZ and 8-fold lower than pH6nZ) but not fully as in the case of pHS3nZ. This is also in agreement with the hypothesis that 40 nucleotides of HIV-1 gag sequences might not be sufficient for efficient vector RNA packaging and this could account for the partial and not complete restoration in titres observed with pHS1nZR in the presence of Rev.

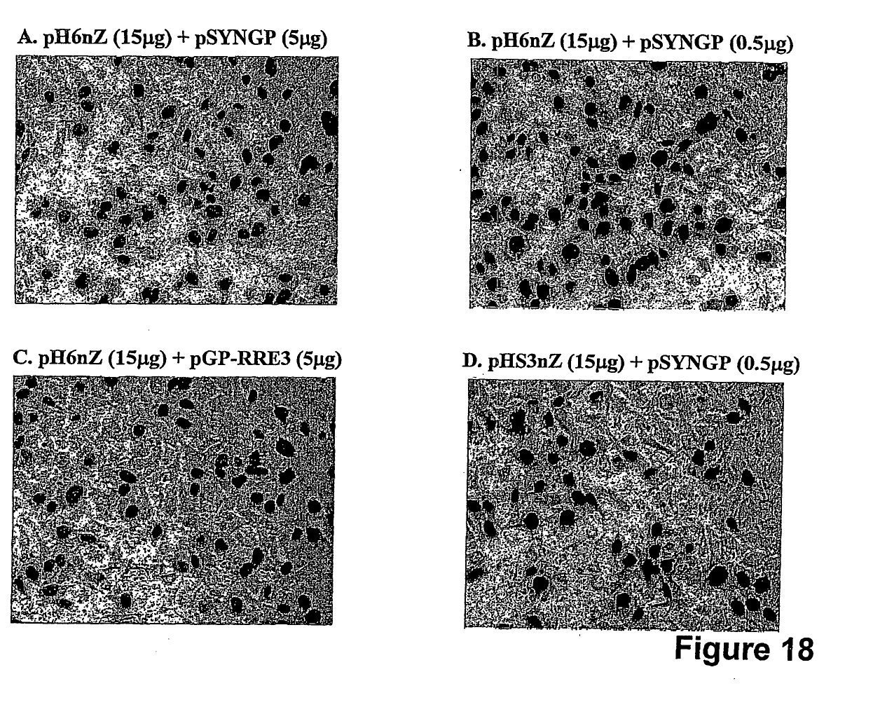

In addition, end-point titres were determined for pHS3nZ and pH6nZ with pSYNGP in HeLa and HT1080 human cell lines. In both cases titres followed the pattern observed in 293T cells, with titres being 2-3 fold lower for pHS3nZ than for pH6nZ (See FIG. 10). Finally, transduction efficiency of vector produced with pHS3nZ or pH6nZ and different amounts of pSYNGP or pGP-RRE3 at different m.o.i.=s (and as high as 1) was determined in HT1080 cells. This experiment was performed as the high level gag-pol expression from pSYNGP may result in interference by genome-empty particles at high vector concentrations. As expected for VSVG pseudotyped retroviral particles (Arai, T., M. Takada, M. Ui, and H. Iba. 1999. Virology. 260:109-115) transduction efficiencies correlated with the m.o.i.=s, whether high or low amounts of pSYNGP were used and with pH6nZ or pHS3nZ. For m.o.i. 1 transduction efficiency was approximately 50-60% in all cases (FIG. 18). The above data indicate that no interference due to genome-empty particles is observed in this experimental system.

The Codon Optimized Gag-Pol Gene Does Not Use the Exportin-1 Nuclear Export Pathway

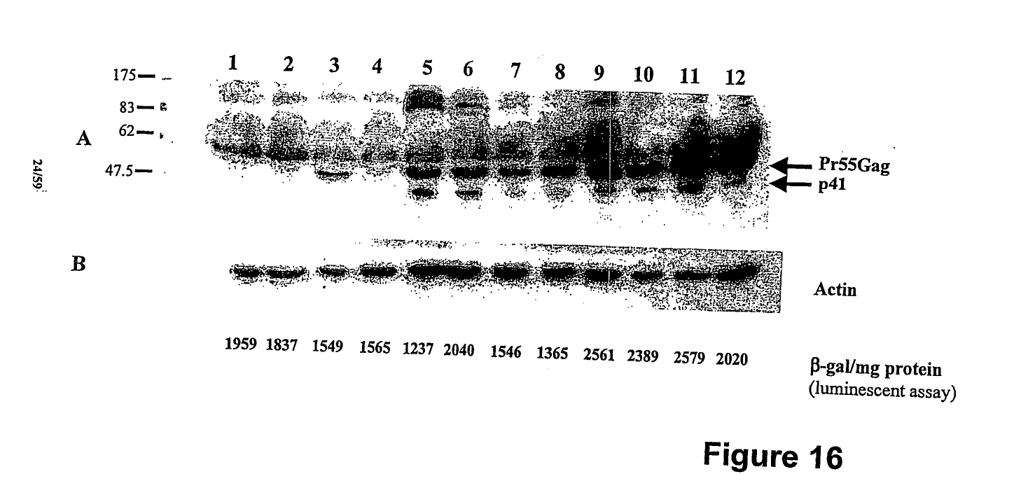

Rev mediates the export of unspliced and singly spliced HIV-1 mRNAs via the nuclear export receptor exportin-1 (CRMI) (Fomerod, M., M. Ohno, M. Yoshida, and I. W. Mattaj. 1997. Cell. 90:1051-1060, Fridell, R. A., H. P. Bogerd, and B. R. Cullen. 1996. proc. Natl. Acad. Sci. USA. 93:4421-4, Pollard, V., and M. Malim. 1998. Annu. Rev. Microbiol. 52:491-532, Stade, K., C. S. Ford, C. Guthrie, and K. Weis. 1997. Cell. 90:1041-1050, Ullman, K. S., M. Powers, A, and D. J. Forbes. 1997. Cell. 90:967-970). Leptomycin B (LMB) has been shown to inhibit leucine-rich NES mediated nuclear export by disrupting the formation of the exportin-1/NES/RanGTP complex (Otero, G. C., M. E. Harris, J. E. Donello, and T. J. Hope. 1998. J. Virol. 72:7593-7597, Pollard, V., and M. Malim. 1998. Annu. Rev. Microbiol. 52:491-532). In particular, LMB inhibits nucleo-cytoplasmic translocation of Rev and Rev-dependent HIV mRNAs (Wolff et al. 1997. Chem Biol. 4: 139-147). To investigate whether exportin-1 mediates the export of the codon optimized gag-pol constructs, the effect of LMB on protein production was tested. Western blot analysis was performed on cell lysates from cells transfected with the gag-pol constructs (+/−pCMV-Rev) and treated or not with LMB (7.5 nM, for 20 hours, beginning treatment 5 hours post-transfection). To confirm that LMB had no global effects on transport, the expression of β-gal from the control plasmid pCMV-βGal was also measured. An actin internal control was used to account for protein variations between samples. The results are shown in FIG. 16. As expected (Wolff et al. 1997. Chem Biol. 4: 139-147), the wild type gag-pol was not expressed in the presence of LMB (compare lanes 3 and 4), whereas LMB had no effect on protein production from the codon optimized gag-pol, irrespective of the presence of the RRE in the transcript and the provision of Rev in trans (compare lanes 5 and 6, 7 and 8, 9 and 10, 11 and 12, 5-6 and 11-12). The resistance of the expression of the codon-optimized gag-pol to inhibition by LMB indicates that the exportin-1 pathway is not used and therefore an alternative export pathway must be used. This offers a possible explanation for the Rev independent expression. The fact that the presence of a nonfunctional Rev/RRE interaction did not affect expression implies that the RRE does not necessarily act as an inhibitory (e.g. nuclear retention) signal per se, which is in agreement with previous observations (Chang, D. D., and P. A. Sharp. 1989. Cell. 59:789-795, Mikaelian, I., M. Krieg, M. Gait, and J. Karn. 1996. J. Mol. Biol. 257:246-264).

In conclusion, this is the first report of an HIV-1 based vector system, composed of pSYNGP, pHS3nZ and pHCMVG, where significant vector production can be achieved in the absence of all accessory proteins. These data indicate that in order to achieve maximum titres the HIV vector genome must be configured to retain efficient packaging and that this requires the retention of gag sequences and a splice donor. By reducing the gag sequence to 360 nt in pHS3nZ and combining this with pSYNGP it is possible to achieve titre of at least 105 I.U./ml that is only 5-fold lower than the maximum levels achieved in the presence of Rev.

### Example 2

### EIAV

Codon-Optimized EIAV Gag-Pol Expression Cassettes

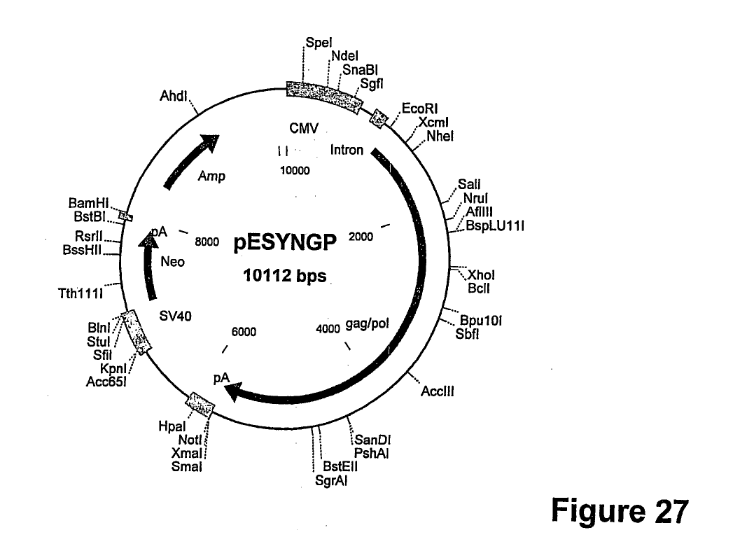

The issue of whether the codon-optimization process would alter the properties of the gag-pol gene of the non-primate lentivirus EIAV was examined. The sequence is of the codon-optimized gene is shown from nt1103 to 5760 of SEQ ID NO:5 (FIG. 9). The wild type and the codon-optimized sequences are denoted WT and CO, respectively. The codon usage was changed to that of highly expressed mammalian genes. pESYNGP (FIG. 27 and SEQ ID NO:5) was made by transferring an XbaI-NotI fragment from a plasmid containing a codon-optimized EIAV gag/pol gene, synthesised by Operon Technologies Inc., Alameda, Calif., into pCIneo (Promega). The gene was supplied in a proprietary plasmid backbone, GeneOp. The fragment transferred to pCIneo includes sequences flanking the codon-optimized EIAV gag/pol ORF: tctagaGAATTCGCCACCATG- EIAV gag/pol- TGAACCCGGGgcggccgc. The ATG start and TGA stop codons are shown in bold and the recognition sequences for XbaI and NotI sites in lower case.

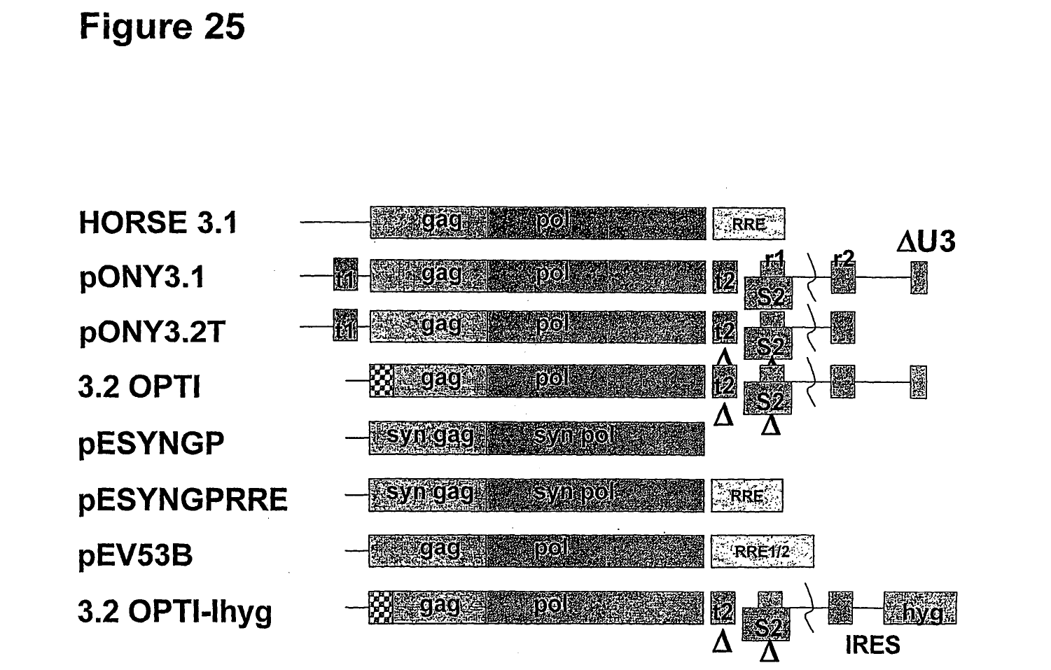

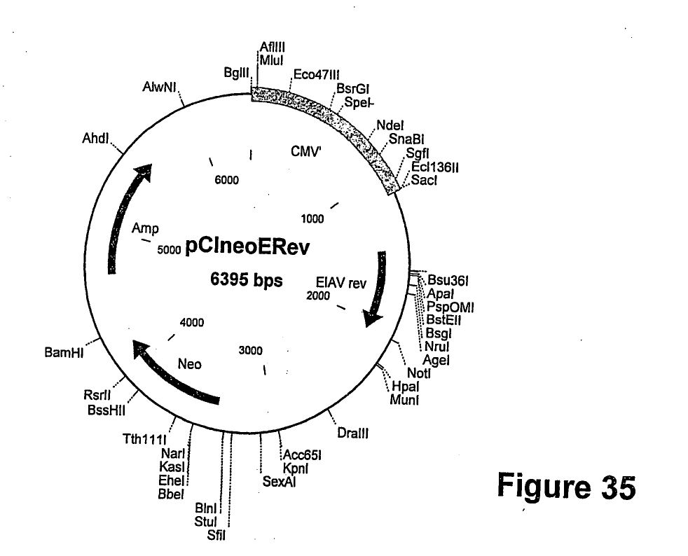

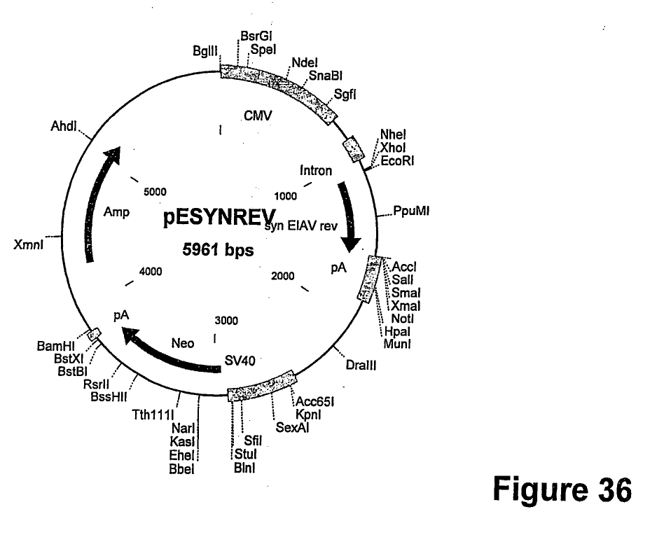

The expression of Gag/Pol from the codon-optimized gene was assessed with respect to that from various wild type EIAV gag/pol expression constructs by transient transfection of HEK 293T cells (FIG. 25). Transfections were carried out using the calcium phosphate technique, using equal moles of each Gag/Pol expression plasmid together with a plasmid which expressed EIAV Rev either from the wild type sequence or from a codon-optimized version of the gene: pClneoEREV (WO 99/32646) (FIG. 35 and SEQ ID NO:13) or pESYNREV (FIG. 36 and SEQ ID NO:14), respectively. pESYNREV is a pClneo-based plasmid (Promega) which was made by introducing the EcoRI to SalI fragment from a synthetic EIAV REV plasmid, made by Operon Technologies Alameda, Calif. The plasmid backbone was the proprietary plasmid GeneOp in which was inserted a codon-optimized EIAV REV gene flanked by EcoRI and SalI recognition sequences and a Kozak consensus sequence to drive efficient translation of the gene. The mass of DNA on each transfection was equalised by addition of pClneo plasmid. In transfections in which a Rev expression plasmid was omitted, a similar mass of pClneo (Promega) was used instead (lanes labelled pCIneo). Cytoplasmic extracts were prepared 48 hours post transfection and 15 μg amounts of protein were fractionated by SDS-PAGE and then transferred to Hybond ECL. The Western blot was probed with a polyclonal antisera from an EIAV-infected horse and then with a secondary antibody, anti-horse horse-radish peroxidase conjugate. Development of the blot was carried out using the ECL kit (Amersham). Positive controls for the blotting and development procedure, and cytoplasmic extract from untransfected HEK 293T cells are as indicated. The positions of various EIAV proteins are indicated.

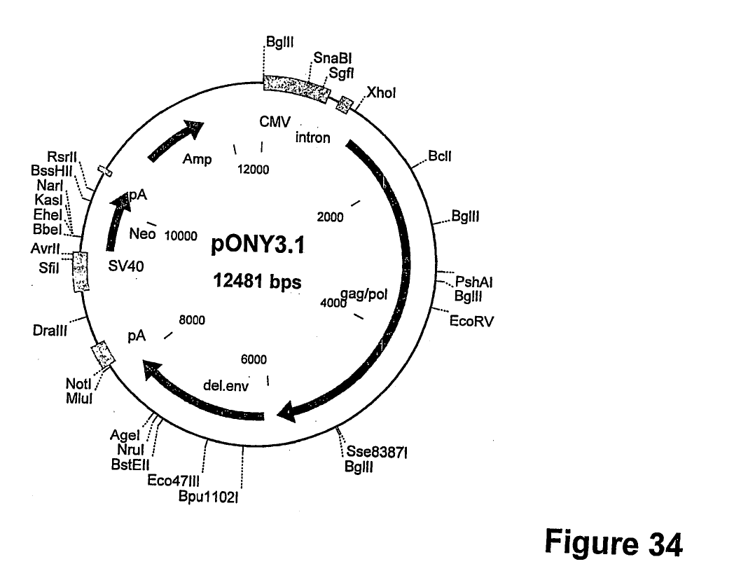

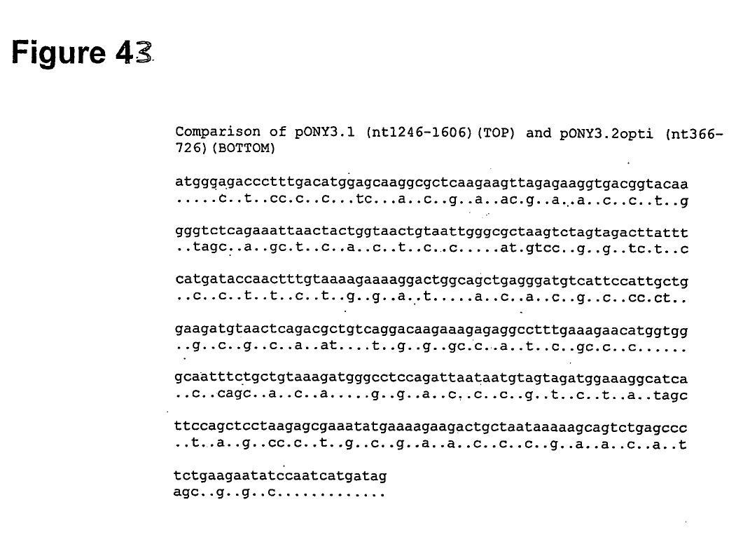

Expression from wild type gag/pol was achieved from various plasmids (see FIG. 25). pONY3.2T is a derivative of pONY3.1 (WO 99/32646) (FIG. 34 and SEQ ID NO:12) in which mutations which ablate expression of Tat and S2 have been made. In addition, the EIAV sequence is truncated downstream of the second exon of rev. Specifically, expression of Tat is ablated by an 83 nt deletion in exon 2 of tat which corresponds with respect to the wild type EIAV sequence, Acc. No. U01866, to deletion of nt 5234-5316 inclusive. S2 ORF expression is ablated by a 5 Int deletion, corresponding to nt 5346-5396 of Acc. No. U01866. The EIAV sequence is deleted downstream of a position corresponding to nt 7815 of Acc. No. U01866. These alterations do not alter rev, hence expression of this gene is expressed as for pONY3.1. pONY3.2 OPTI is a derivative of pONY3.1 which has the same deletions for ablation of Tat and S2 expression as described above. In addition, the first 372 nt of gag have been ‘codon-optimized’ for expression in human cells. The sequence of the wild type and codon-optimized sequences present in pONY3.2 OPTI in this region are compared in FIG. 43. Base differences between the sequences are indicated. The region which was codon-optimized represents the region of overlap between the vector and wild-type gag/pol expression constructs. Reduction of homology within this region would be expected to improve the safety profile of the vector system due to the reduced chances of recombination between the vector genome and the gag/pol transcripts. 3.2 OPTI-Ihyg is a derivative of 3.2 OPTI in which the SnaBI-NotI fragment of 3.2 OPTI is transferred to pIRESIhygro (Clontech) prepared for ligation by digestion with the same sites. The gag/pol gene is thus placed upstream of the IRES hygromycin phosphotransferase. Of note is the fact that the resulting construct contains the intron from pCIneo, not from pIRES1hygro. pEV53B is a derivative of PEV53A (WO98/51810) in which the EIAV-derived sequence upstream of the Gag initiation codon is reduced to include only the major splice donor and surrounding seqeunces: CAG/GTAAGATG, where the Gag initiation codon is shown in bold face.

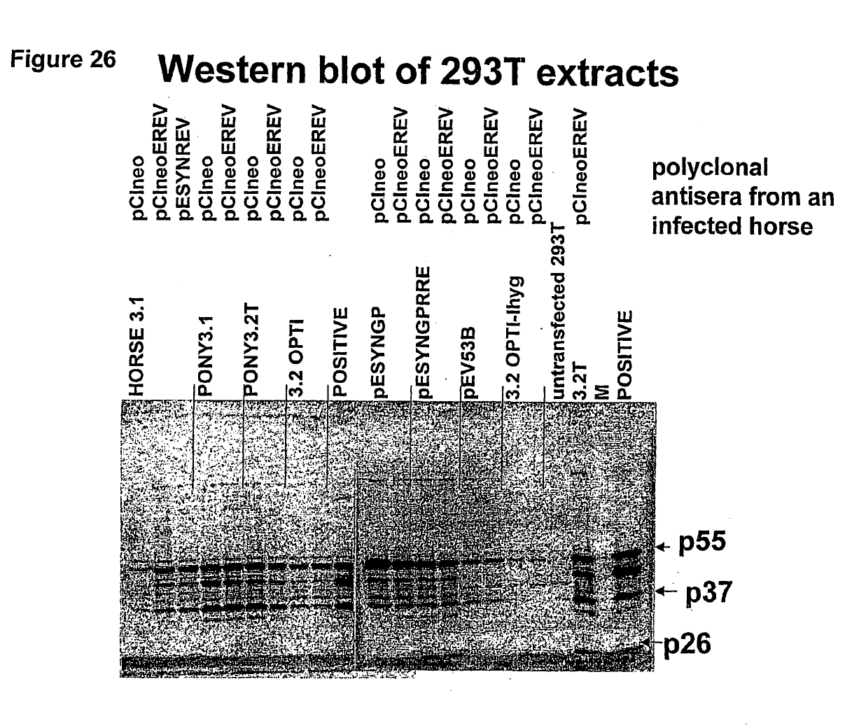

The results (FIG. 26) shown the Rev-dependence of Gag/Pol expression from pHORSE3.1 (WO 99/32646), which has an EIAV derived leader sequence starting just downstream of the primer binding site and an RRE placed downstream of gag/pol composed of the two EIAV sequences reported to have RRE activity. Expression was enhanced by the same amount when Rev expression was driven by wild type (pClneoERev) (FIG. 35) or codon-optimized (pESYNREV) (FIG. 36) genes. This result confirms the functionality of the codon-optimized Rev expression plasmid.

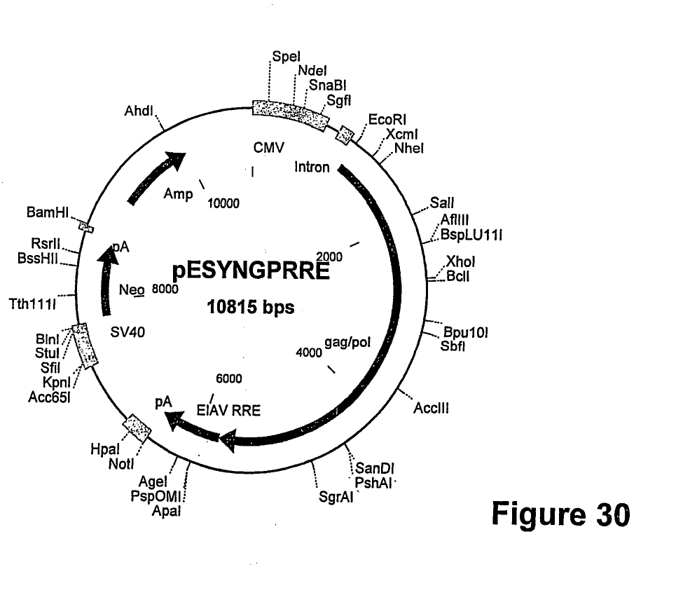

In contrast to expression of Gag/Pol from pONY3.1, expression from pESYNGP was not influenced by the presence of Rev, however it was slightly lower than from pONY3.1 or pON3.2T. Expression from pESYNGPRRE (FIG. 30 and SEQ ID NO:7), in which the EIAV RRE sequence present in pHORSE3.1 is placed downstream of gag/pol, appeared slightly lower than from pESYNGP. The levels of expression from 3.2 OPTI and 3.2 OPTI-Ihyg were significantly lower than from pESYNGP or pONY3.1, even in the presence of Rev. This result suggested that there may be determinants of Gag/Pol expression within the first 372 nt of the gag and showed that 3.2 OPTI was unlikely to be useful as a basis for EIAV vector production. Furthermore it demonstrates that codon-optimization of only certain regions of the whole gag/pol gene may not lead to high levels of Rev-independent expression.

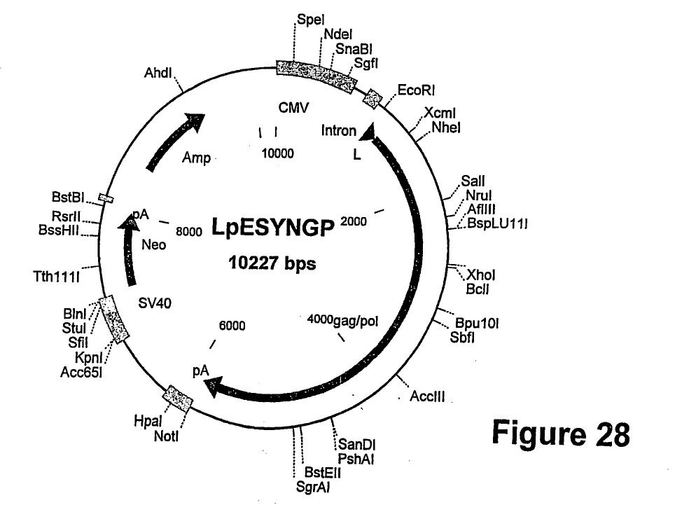

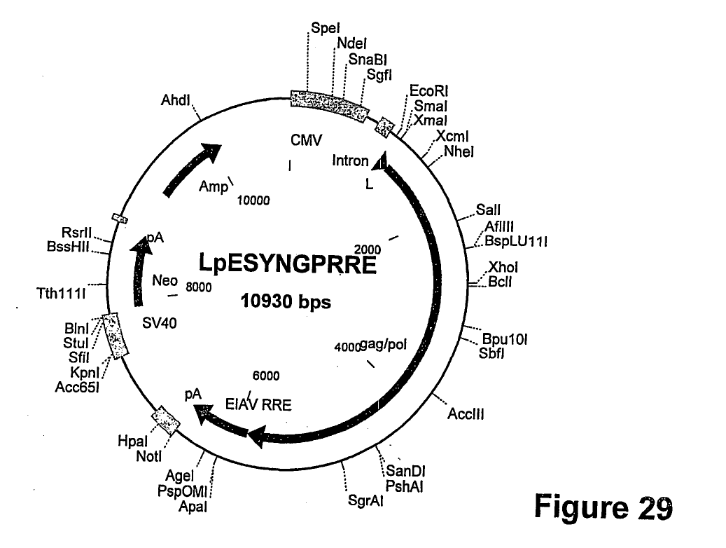

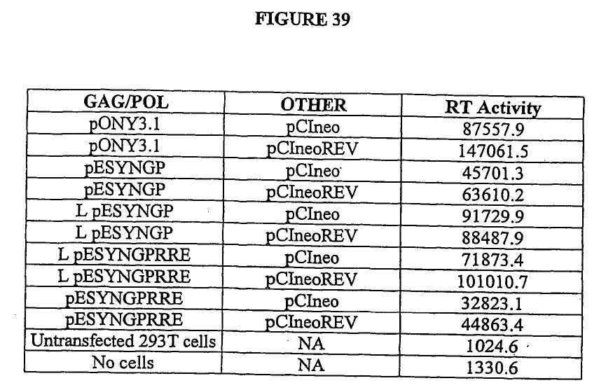

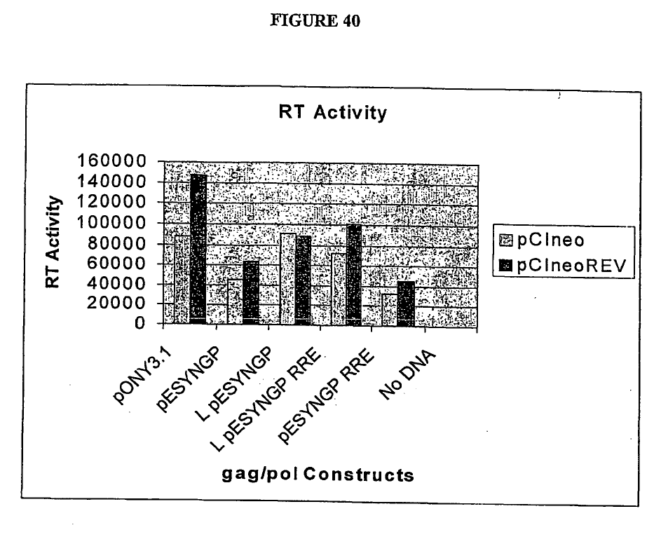

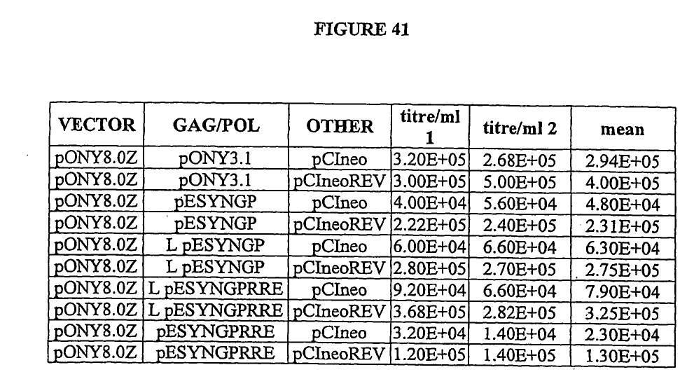

It was previously demonstrated (Mitrophanous K, Yoon S, Rohil J, Patil D, Wilkes F, Kim V, Kingsman S, Kingsman A, Mazarakis N, 1999. Gene Ther. 6 (11): 1808-18) that the 5′ leader (121 bp upstream of the ATG start codon) and the RRE sequence (Mitrophanous K, Yoon S, Rohil J, Patil D, Wilkes F, Kim V, Kingsman S, Kingsman A, Mazarakis N, 1999. Gene Ther. 6 (11): 1808-18) are important for high expression of the wild type EIAV gag-pol. Three constructs were made that contained either the leader sequence (LpESYNGP), the leader and RRE sequences (LpESYNGPRRE) or the RRE sequence (pESYNGPRRE). The sequences of these constructs are shown in SEQ ID NOS:6-8 and FIGS. 28-30. They were transfected into 293T cells in either the presence or absence of Rev expression plasmid. The cell supernatant was then measured for reverse transcriptase activity (RT), using a conventional RT assay, to evaluate which construct generated the highest amount of gag-pol mRNA. The results are shown in FIGS. 39 and 40. It is clear from these results that the 5′ leader leads to an increase in RT activity. The ability of these Gag/Pol expression constructs to support formation of infectious vector particles was also tested by transient transfection of HEK 293 cells. The results of this analysis of show that all of the constructs could provide functional EIAV Gag/Pol, and show the Rev dependence of titre with the pONY8.0Z vector genome plasmid, which does not encode any EIAV proteins (FIG. 41).

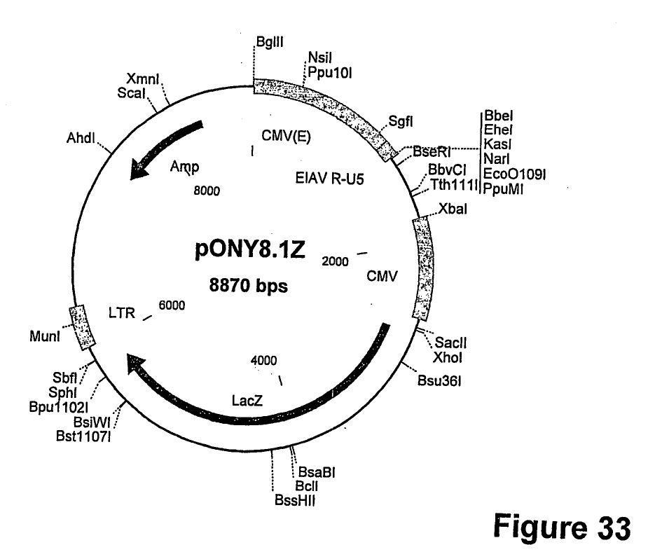

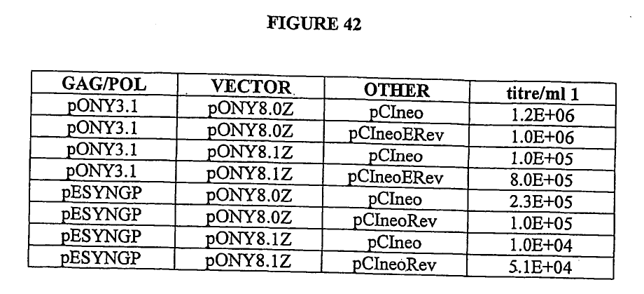

The ability of pESYNGP to act in concert with a minimal EIAV vector genome plasmid pONY8.1Z (FIG. 33, SEQ ID NO:11) was evaluated (FIG. 42). The result shows that the titres obtained with pESYNGP and pONY8.1Z are about 10-fold lower than from pONY3.1 and pONY8.1Z. This reduced titre reflects the lack of Rev protein in the system rather than a deficiency of Gag/Pol production, which was already shown as being independent of Rev expression.

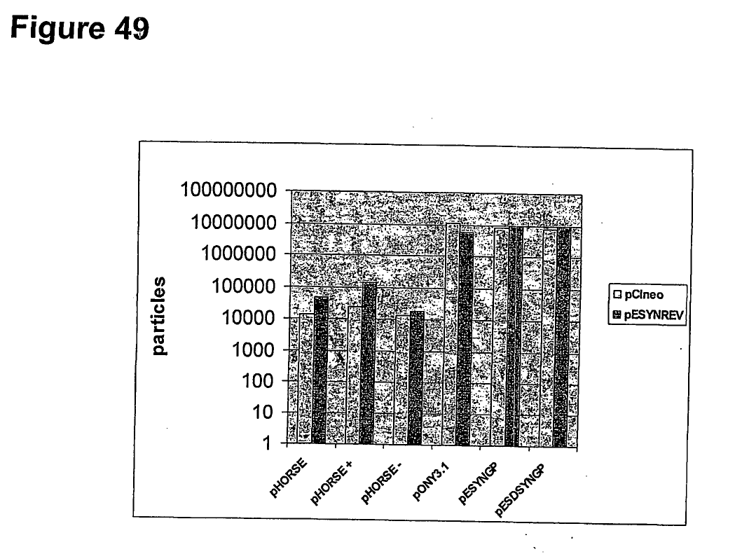

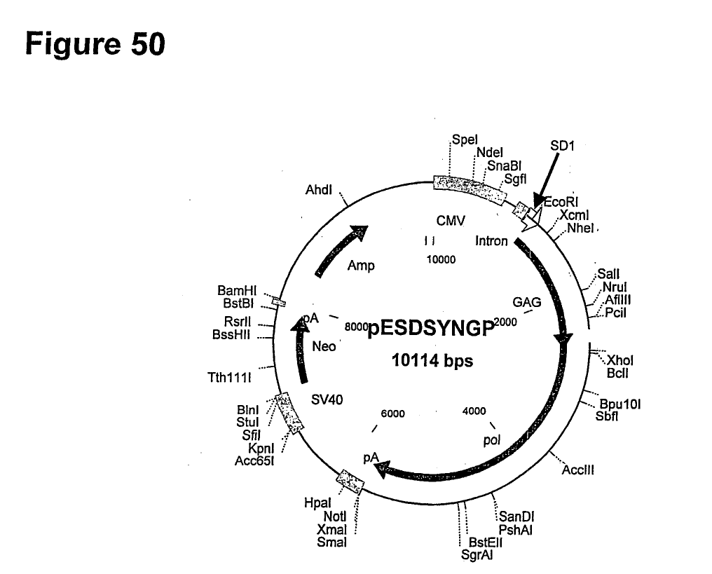

Expression of EIAV Gag/Pol was also tested from pESDSYNGP (FIG. 50 and SEQ ID NO:18) in which the Kozak consensus sequence of Gag is replaced by the natural EIAV splice donor. pESDSYNGP was made from pESYNGP by exchange of the 306 bp EcoRI-NheI fragment, which runs from just upstream of the start codon for gag/pol to approximately 300 base pairs inside the gag/pol ORF with a 308 bp EcoRI-NheI fragment derived by digestion of a PCR product made using pESYNGP as template and using the following primers: SD FOR [GGCTAGAGAATTCCAGGTAAGATGGGCGATCCCCTCACCTGG] and SD REV [TTGGGTACTCCTCGCTAGGTTC]. This manipulation replaces the Kozak concensus sequence upstream of the ATG in pESYNGP with the splice donor found in EIAV. The sequence between the EcoRI site and the ATG of gag/pol is thus CAGGTAAG, exactly as found in the natural viral sequence. Therefore the mRNA is deleted with respect to sequences upstream but not downstream of the splice donor. The performance of pESDSYNGP was assessed relative to pESYNGP and other expression plasmids by measurement of reverse transcriptase activity in supernatants from transiently transfected HEK 293T cells using a Taqman-based version of the product enhanced reverse transcriptase (PERT) assay. In this method, reverse transcriptase associated with vector particles is released by mild detergent treatment and used to synthesize cDNA using MS2 bacteriophage RNA as template. MS2 RNA template and primer are present in excess hence the amount of cDNA is proportional to the amount of RT released from the particles. Therefore, the amount of cDNA synthesised is proportional to the number of particles. MS2 cDNA is then quantitated using Taqman technology. The assay is carried out on test samples in parallel with a vector stock of known titre and estimated particle content. The use of the standard allows creation of a ‘standard curve’ and allows the relative RT content of various samples to be calculated. The results of this analysis are shown in FIG. 49. The results show that Gag/Pol expression is virtually identical from pESYNGP and pESDSYNGP. The results also indicate that expression is not significantly enhanced by Rev. The activity of the Rev expression plasmid is confirmed by the result obtained with pHORSE+, in which there is an RRE downstream of the wild type EIAV gag/pol, and that shows a 6-fold enhancement of expression in the presence of Rev. It was also noted that the expression from pHORSE was enhanced 3-fold in the presence of Rev. Since this construct has no RRE it suggests that Rev may be having a non-specific enhancing effect on expression, possibly as a result of being expressed at high levels in this experimental system.

The ability of pESYNGP to participate in the formation of infectious viral vector particles, when co-transfected with plasmids for the vector genome and envelope was assessed by transient transfection of HEK 293T, as described previously (Kim, V. N., K. Mitrophanous, S. M. Kingsman, and K. A. J. 1998. J Virol 72: 811-816, Soneoka, Y., P. M. Cannon, E. E. Ramsdale, J. C. Griffiths, G. Romano, S. M. Kingsman, and A. J. Kingsman. 1995. Nucleic Acids Res. 23:628-33). Briefly, 293T cells were seeded on 6 cm dishes (1.2×10⁶/dish) and 24 hours later they were transfected by the calcium phosphate procedure. The medium was replaced 12 hours post-transfection and supernatants were harvested 48 hours post-transfection, filtered (0.45 μm filters) and titered by transduction of D17, canine osteosarcoma cells, in the presence of 8 μg/ml Polybrene (Sigma). Cells were seeded at 0.9×10⁵/well in 12 well plates 24 hours prior to use in titration assays. Dilutions of supernatant were made in complete media (DMEM/10% FBS) and 0.5 ml aliquots plated out onto the D17 cells. 4 hours after addition of the vector the media was supplemented with a further 1 ml of media. Transduction was assessed by X-gal staining of cells 48 hours after addition of viral dilutions.

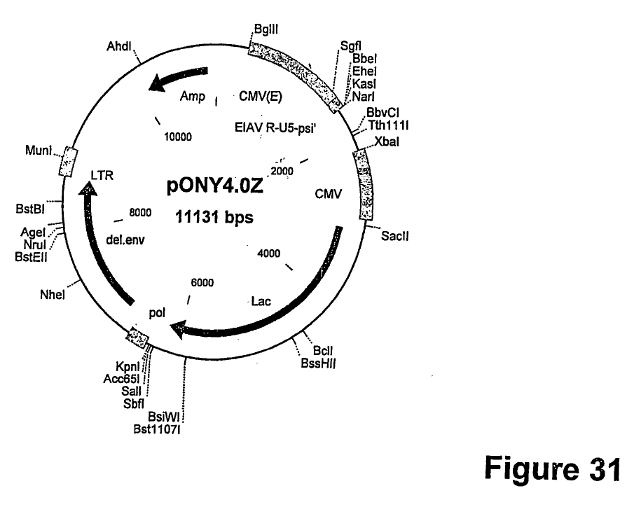

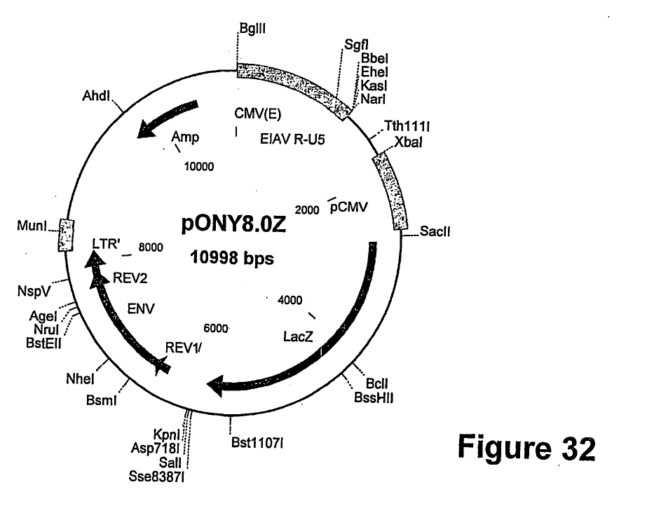

The vector genomes used for these experiments were pONY4.0Z (FIG. 31 and SEQ ID NO:9) and pONY8.0Z (FIG. 32 and SEQ ID NO:10).

pONY4.0Z (WO99/32646) was derived from pONY2.11Z by replacement of the U3 region in the 5′LTR with the cytomegalovirus immediate early promoter (pCMV). This was carried out in such a way that the first base of the transcript derived from this CMV promoter corresponds to the first base of the R region. This manipulation results in the production of high levels of vector genome in transduced cells, particularly HEK 293T cells, and has been described previously (Soneoka, Y., P. M. Cannon, E. E. Ramsdale, J. C. Griffiths, G. Romano, S. M. Kingsman, and A. J. Kingsman. 1995. Nucleic Acids Res. 23:628-33). pONY4.0Z expresses all EIAV proteins except for envelope, expression of which is ablated by a deletion of 736 nt between the HindIII sites present in env.
pONY8.0Z was derived from pONY4.0Z by introducing mutations which 1) prevented expression of TAT by an 83 nt deletion in the exon 2 of tat) prevented S2 ORF expression by a 51 nt deletion 3) prevented REV expression by deletion of a single base within exon 1 of rev and 4) prevented expression of the N-terminal portion of gag by insertion of T in ATG start codons, thereby changing the sequence to ATTG from ATG. With respect to the wild type EIAV sequence Acc. No. U01866 these correspond to deletion of nt 5234-5316 inclusive, nt 5346-5396 inclusive and nt 5538. The insertion of T residues was after nt 526 and 543.

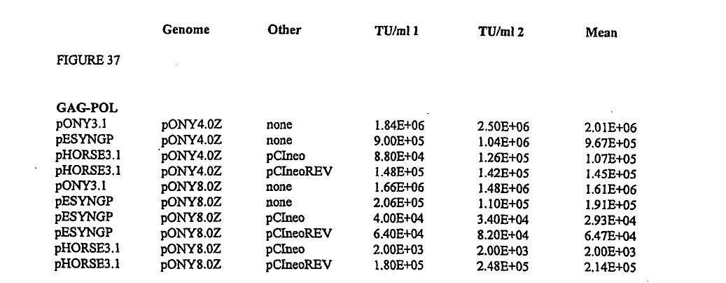

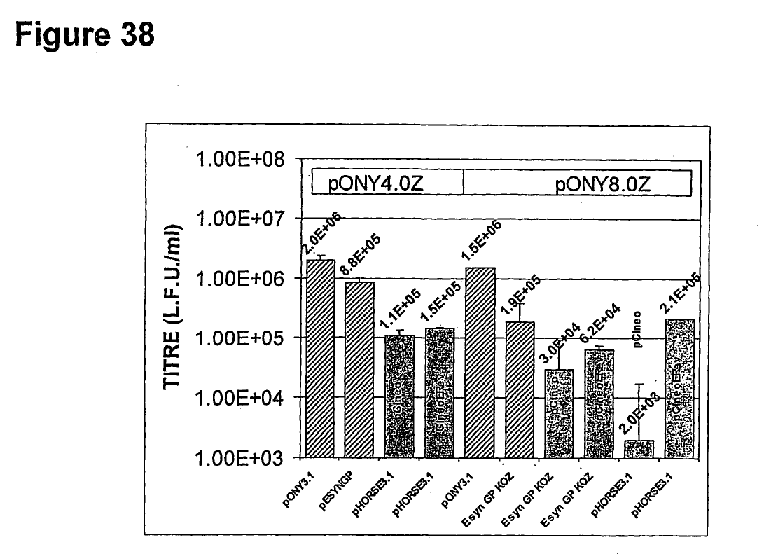

The results of this analysis are shown tabulated in FIG. 37, and graphically in FIG. 38. Transfections were carried out with only 3 plasmids (vector genome, gag/pol expression plasmid and VSV-G expression plasmid)—diagonal lined bars, or with four plasmids, which included the previous set of plasmids together with an additional plasmid encoding Rev or a similar plasmid not coding a functional protein—filled bars. The results show that high titres of vector can be achieved using pESYNGP to supply EIAV Gag/Pol. The highest titres were obtained using the Rev-expressing vector genome plasmid, pONY4.0Z, and they were only slightly lower than observed when Gag/Pol was supplied by pONY3.1. Lower titres were observed with pONY8.0Z vector genome plasmid with pESYNGP than with pONY3.1. This is due to the Rev expression requirement of pONY8.0Z. Rev is expressed by pONY3.1, but not pESYNGP. These results confirm the utility of the codon-optimized Gag/Pol expression plasmid.

Use of the Synthetic EIAV gag/pol Gene in Construction of Cell Lines Which Stably Express EIAV gag/pol.

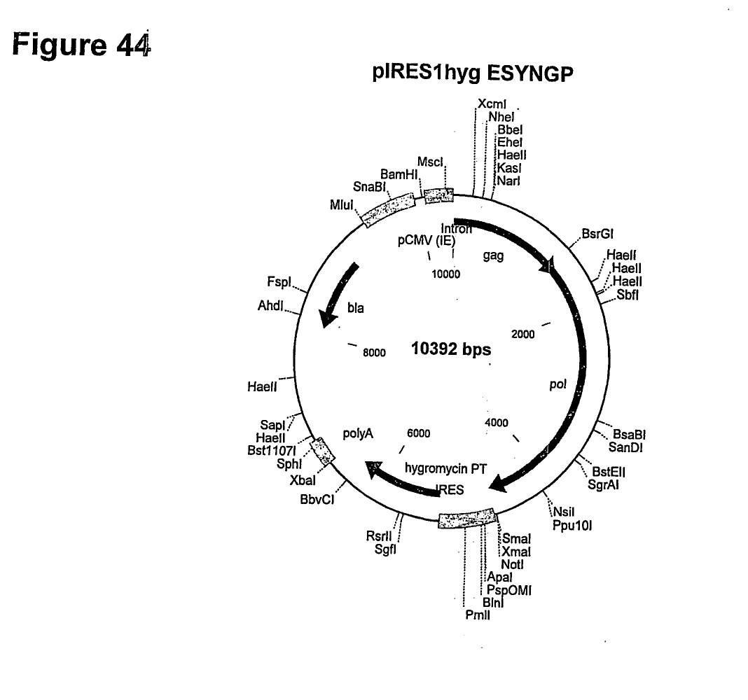

Cells lines which express high amounts of EIAV Gag/pol are required for the construction of packaging and producer cells for EIAV vectors. As a first step in their construction HEK 293 cells were stably transfected with pIRES1hyg ESYNGP (FIG. 44 and SEQ ID NO:17), in which EIAV Gag/pol expression is driven by a CMV promoter, and is linked to an ORF for expression of hygromycin phosphotransferase by an EMCV IRES. pIRESIhyg ESYNGP was made as follows. The synthetic EIAV gag/pol gene and flanking sequences was transferred from pESYNGP into pIRES1hygro expression vector (Clontech). First, pESYNGP was digested with EcoRI, and the ends filled in by treatment with T4DNA polymerase and then digested with NotI. pIRES1hygro was prepared for ligation with this fragment by digestion with NsiI, the ends trimmed flush by treatment with T4 DNA polymerase, then digested with NotI. Prior to transfection into HEK 293 cells pIRES1hyg ESYNGP was digested with AhdI which linearises the plasmid.

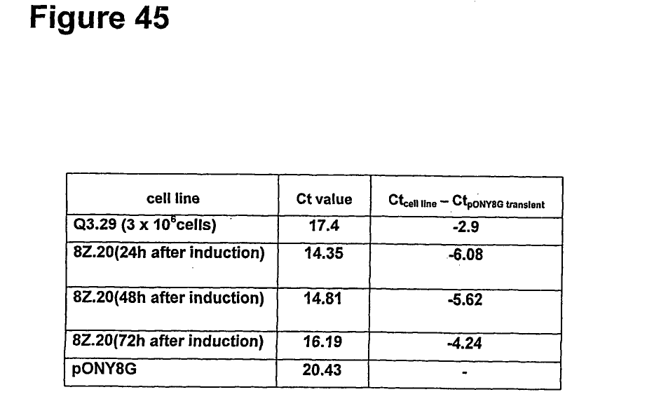

Clonal cell lines were derived by serial dilution and analysed for expression of Gag/Pol by a Taqman-based product enhanced reverse transcriptase (PERT) assay. Data for the cell line Q3.29, which expressed the highest level of Gag/Pol is shown. The analysis showed that the level of expression from the codon-optimized EIAV Gag/Pol cassette in Q3.29 was very similar to that seen for an EIAV producer line, 8Z.20, in which Gag/Pol is expressed from the pEV53B wild type expression cassette, that produced vector particles at titres of almost 10⁶ transducing units per ml. (FIG. 45). Assuming exponential amplification during the assay, a difference of Ct value of 1.0 corresponds to a difference of 2-fold in concentration of the reverse transcriptase released from the particles. Therefore the difference in Gag/Pol expression between Q3.29 and 8Z.20 cells is approximately 2-8 fold. Furthermore the Ct values observed indicate that the level of expression of Gag/Pol is significantly higher than in samples of pONY8G vector particles with a titre of 2×10⁶ transducing units per ml on D17 cells, but made by transient transfection of HEK 293T cells. These data indicate that the codon-optimized EIAV Gag/Pol construct can be used in the construction of EIAV packaging and producer lines and confirms the previous result that expression is independent of Rev expression.

The Q3.29 cell line was then tested for its ability to support production of infectious vector particles when transfected with a vector genome plasmid, pONY8.0Z, and the VSV-G envelope expression plasmid, pRV67 and the EIAV REV expression plasmid, pESYNREV. In addition, the performance of a plasmid pONY8.3G FB29 (−), which is a modified form of the pONY8G vector genome plasmid, was evaluated. PONY8G is a standard EIAV vector genome used for comparison purposes. The modifications and construction of pONY8.3G FB29 (−) (SEQ ID NO:19) are described in PCT/GB00/03837 and briefly are 1) the introduction of loxP recognition sites upstream and downstream of the vector genome cassette 2) the placement of an expression cassette for codon-optimized REV, derived from pESYNREV, and driven by the FB29 U3 promoter downstream of the vector genome cassette and orientated so that the direction of transcription was towards the vector genome cassette. The REV expression cassette is located upstream of the 3′ loxP site. Thus the pONY8.3G FB29-plasmid carries expression cassettes for the vector genome RNA and for EIAV Rev.

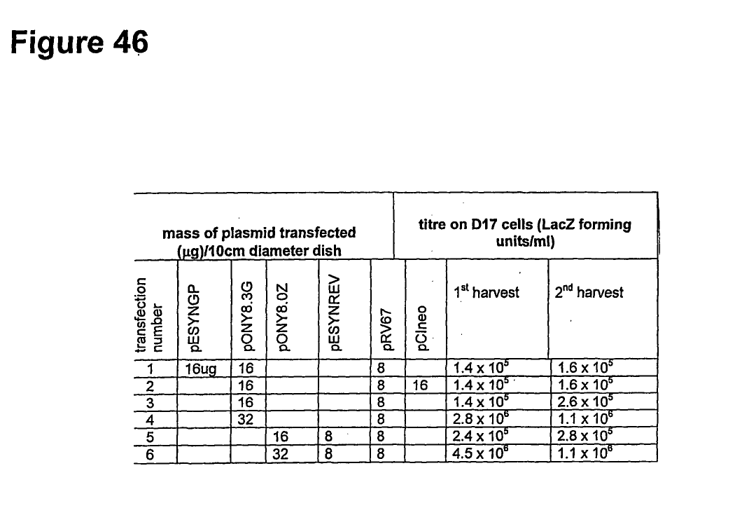

The titres were established by limiting dilution on D17 canine osteosarcoma cells and are shown in FIG. 46.

The titres obtained from transfections 2-6 were up to 4.5×10⁶ transducing units per ml indicating levels of Gag/Pol expression sufficient to support titres at least this high. The titres obtained were not higher when additional Gag/Pol was supplied (transfection 1) indicating that Gag/Pol expression was not the limitation on titre.

Improved Safety Profile Due to Gag/Pol Expression From a Codon-Optimized Expression Construct

RCR formation takes place by recombination between different components of the vector system or by recombination of vector system components with nucleotide sequences present in the producer cells. Although recombination at the DNA level during construction of producer cell lines is possible (perhaps leading to insertional activation of endogenous retroelements or retroviruses) it is thought that recombination to produce RCR occurs mainly between RNA's undergoing reverse transcription, hence occurs within the mature vector particles. In consequence, recombination will be more likely to occur between RNA's which contain packaging signals, such as the vector genome and the gag/pol mRNA. Usually however the gag/pol transcript is modified so that it is deleted with respect to some or all defined packaging elements, thereby reducing the chances of its involvement in recombination.

The codon-optimization process used to create the HIV and EIAV Gag/Pol expression plasmid, pSYNGP and pESYNGP, also results in disruption of sequences and structures that direct packaging as a result of introducing changes at approximately every 3^{rd} nucleotide position. Evidence for the lower level of incorporation of the codon-optimized RNA derived from pESYNGP into virions was obtained.

The packaging of mRNA's derived from a wild type gag/pol pEV53B expression cassette, and from the codon-optimized EIAV gag/pol expression cassette, pESYNGP, was compared. Medium was collected from a HEK 293 based cell-lines which were stably transfected with either pEV53B (cell line B-241), or with pESYNGP. Both cell lines produce vector particles which do not contain vector RNA and do not have envelopes. In some experiments, an EIAV vector genome plasmid (pECG3-CZW) was transfected into the cells to serve as an internal positive control for hybridisation and for the presence of particles capable of packaging RNA. pECG3-CZW is a derivative of pEC-LacZ (WO98/51810) and was made from the latter by I) reduction of gag sequences so that only the first 200 nt of gag, rather than the first 577 nt, was included and 2) inclusion of the woodchuck hepatitis virus post-transcriptional regulatory element (WHV PRE) (corresponding to nt 901-1800 of Acc. No. J04514) into the NotI site downstream of the LacZ reporter gene.

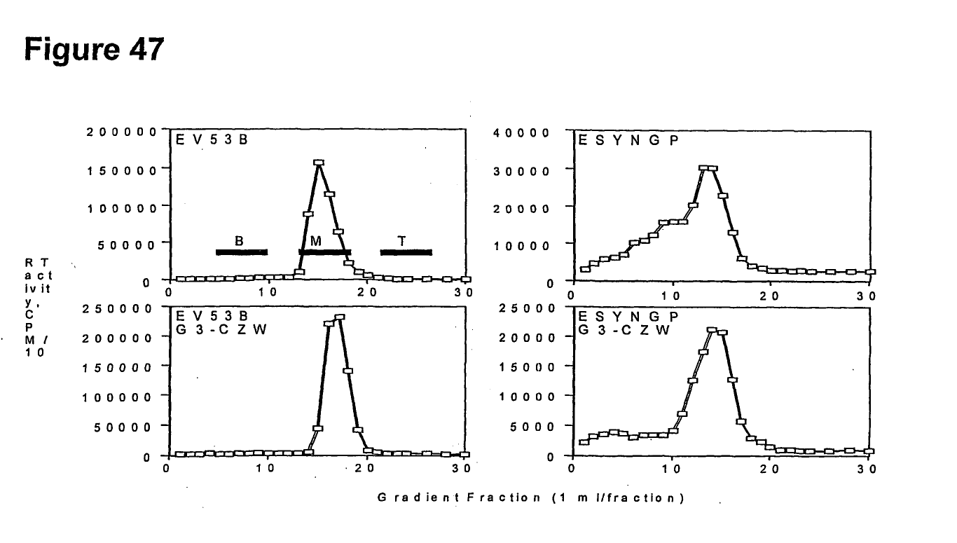

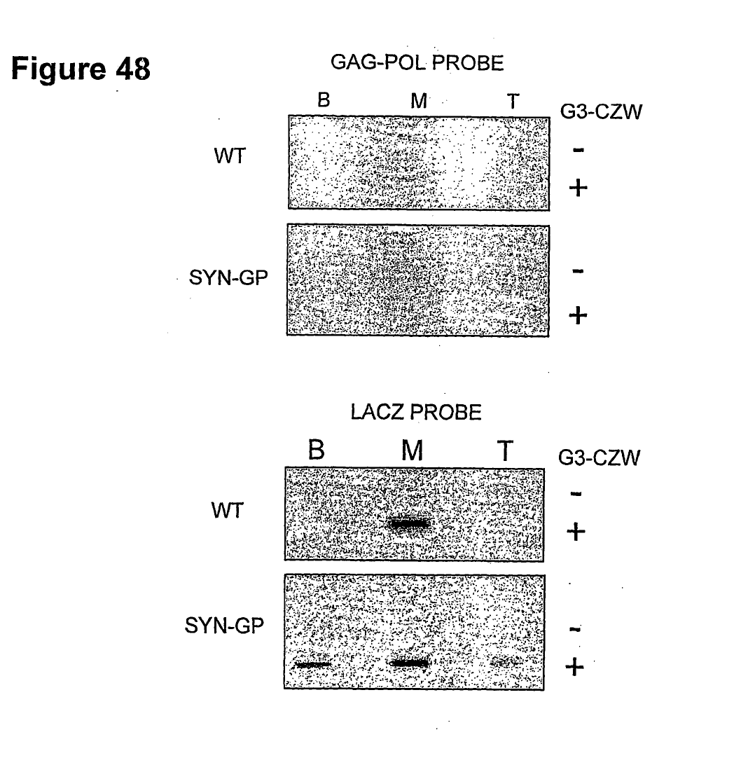

Viral particles derived from each of the cell lines were then partially purified from the medium by equilibrium density gradient centrifugation. To do this 10 ml of medium from producer cells, harvested at 24 hours after induction with sodium butyrate, was layered onto a 20-60% (w/w) sucrose gradient in TNE buffer (pH 7.4) and centrifuged for 24 hours at 25,000 rpm and 4° C. in a SW28 rotor. Fractions were collected from the bottom and 10 μl of each fraction assayed for reverse transcriptase activity to locate viral particles. The results of this analysis are shown in (FIG. 47) where the profile of reverse transcriptase activity is shown as a function of gradient fraction. In these figures, the top of the gradient is on the right. It should be noted that the levels of RT activity from the pESYNGP-expressing cell were significantly lower than from pEV53B expressing cells. To determine the RNA content of the purified virions, aliquots from the top, middle or bottorh fractions were pooled (as indicated by the bars labeled T, M and B) and the RNA from each fraction was subjected to slot-blot hybridization analysis. Using a probe specific for a common region of wild type and synthetic gag/pol, encapsidation of RNA was easily detectable in the peak fractions (M) of virions synthesized from the wild type construct (pEV53B), but was not detected from virions synthesized from the synthetic Gag/Pol construct (pESYNGP)(FIG. 48). The control for the presence of capsid capable of carrying out encapsidation was the EIAV G3-CZW vector genome which was readily detected in peak fractions from cells expressing either the wild type or synthetic gag/pol proteins. Even taking into account the different levels of expression from the wild type and synthetic Gag/Pol expression constructs this result indicates that the RNA from the codon-optimized gag/pol gene is packaged significantly less efficiently than the wild type gene and represents a significant improvement to the safety profile of the system. Of further note is that the RNA transcribed from pEV53B was packaged. This RNA is deleted with respect to sequences upstream of the splice donor sequence (CAG/GTAAG) and yet was still packaged. This points to the localization of major packaging determinants within the gag coding region and is in contrast to the collected observations on the location of the packaging signal of HIV-1.

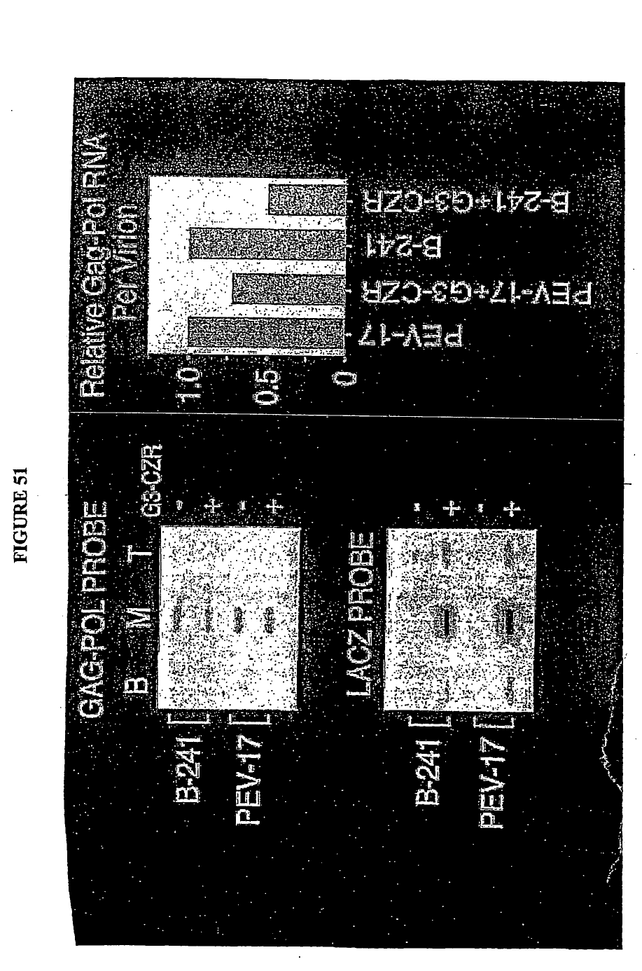

In additional experiments it has been shown that the packaging of transcripts from pEV53B is only slightly lower than from pEV53A (FIG. 51). This indicates further that major packaging sequences are located within the gag coding region. In these experiments cell line B-241 expressed pEV53B RNA and PEV-17 expressed pEV53A RNA. The EIAV vector genome used to confirm the presence of packaging competent vector particles was G3-CZR, which is the same as G3-CZW, described above, except for the replacement of the woodchuck post-transcriptional regulatory element with a sequence containing the EIAV RRE elements. Methodology was as described above.

All publications cited herein are incorporated by reference. Various modifications and variations of the described methods and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

### REFERENCES

1. Miller, N., and J. Whelan. 1997. Hum Gene Ther. 8:803-15.
2. Lewis & Emerman. 1993. J. Virol. 68:510.
3. Naldini, L., U. Blomer, P. Gallay, D. Ory, R. Mulligan, F. H. Gage, I. M. Verma, and D. Trono. 1996. Science. 272:263-7.
4. Morgenstern & Land. 1990. Nucleic Acids Res. 18: 3587-3596.
5. Chang, D. D., and P. A. Sharp. 1989. Cell. 59:789-795.
6. Wang & Semenza. 1993. proc Natl Acad. Sci. 90:430.
7. Dachsetal. 1997. Nature Med. 5:515.
8. Firth et al. 1994. proc Natl Acad. Sci. 90: 6496-6500.
9. Madan et al. 1993. proc Natl Acad. Sci. 90:3928.
10. Semenza & Wang. 1992. Mol Cell Biol. 1992. 12: 5447-5454.
11. Takenaka et al. 1989. J. Biol. Chem. 264: 2363-2367.
12. peshavaria & Day. 1991. Biochem J. 275: 427-433.
13. Inou et al. 1989. J. Biol. Chem. 264: 14954-14959.
14. Overell et al. 1988. Mol Cell Biol. 8: 1803-1808.
15. Attenello & Lee. 1984. Science. 226: 187-190.
16. Gazit et al. 1985. Cancer Res. 55: 1660-1663.
17. Yu et al. 1986. proc Natl Acad. Sci. 83: 3194-3198.
18. Dougherty & Temin. 1987. proc Natl Acad. Sci. 84: 1197-1201.
19. Hawley et al. 1987. proc Natl Acad. Sci. 84: 2406-2410.
20. Yee, J. K., A. Miyanohara, P. LaPorte, K. Bouic, J. C. Burns, and T. Friedmann. 1994. proc. Natl. Acad. Sci. USA. 91:9564-8.
21. Jolley et al. 1983. Nucleic Acids Res. 11: 1855-1872.
22. Emennan & Tenim. 1984. Cell. 39: 449-467.
23. Herman & Coffin. 1987. Science. 236: 845-848.
24. Benderetal., 1987, J Virol 61: 1639-1646.
25. pear et al., 1993, Proc Natl Acad Sci 90: 8392-8396.
26. Danos & Mulligan. 1998. proc Natl Acad. Sci. 85: 6460-6464.
27. Markowitz et al. 1988. Virology. 167: 400-406.
28. Cosset et al., 1995, J. Virol. 69: 7430-7436.
29. Mebatsion et al. 1997. Cell. 90: 841-847.
30. Marshall. 1998. Nature Biotechnology. 16: 129.
31. Nature Biotechnology. 1996. 14:556.
32. Wolff & Trubetskoy. 1998. Nature Biotechnology. 16: 421.
33. DuBridge, R. B., P. Tang, H. C. Hsia, P.-M. Leong, J. H. Miller, and M. P. Calos. 1987. Mol. Cell. Biol. 7:379-387.
34. Gey, G. O., W. D. Coffman, and M. T. Kubicek. 1952. Cancer res. 12:264.
35. Kim, S. Y., R. Byrn, J. Groopman, and D. Baltimore. 1989. J. Virol. 63:3708-3713.
36. Adachi, A., H. Gendelman, S. Koenig, T. Folks, R. Willey, A. Rabson, and M. Martin. 1986. J. Virol. 59:284-291.
37. Haas, J., E.-C. Park, and B. Seed. 1996. Current Biology. 6:315.
38. Zolotukhin, S., M. Potter, W. W. Hauswirth, J. Guy, and N. Muzyczka. 1996. A “humanized” green fluorescent protein cDNA adapted for high-level expression in mammalian cells. J. Virol. 70:4646-54.
39. Fisher, A., E. Collalti, L. Ratner, R. Gallo, and F. Wong-Staal. 1985. Nature. 316:262-265.
40. Kozak, M. 1992. [Review]. Annu. Rev. Cell Biol. 8:197-225.
41. Cassan, M., N. Delaunay, C. Vaquero, and J. P. Rousset. 1994. J. Virol. 68:1501-8.
42. parkin, N. T., M. Chamorro, and H. E. Varmus. 1992. J. Virol. 66:5147-51. 68:3888-3895.
43. Mitrophanous K, Yoon S, Rohil J, Patil D, Wilkes F, Kim V, Kingsman S, Kingsman A, Mazarakis N, 1999. Gene Ther. 6 (11): 1808-18
44. Ory, D. S., B. A. Neugeboren, and R. C. Mulligan. 1996. proc Natl Acad Sci USA. 93:11400-6.
45. Zhu, Z. H., S. S. Chen, and A. S. Huang. 1990. J Acquir Immune Defic Syndr. 3:215-9.
46. Chesebro, B., K. Wehrly, and W. Maury. 1990. J. Virol. 64:4553-7.
47. Spector, D. H., E. Wade, D. A. Wright, V. Koval, C. Clark, D. Jaquish, and S. A. Spector. 1990. J. Virol. 64:2298-2308.
48. Valsesia Wittmann, S., A. Drynda, G. Deleage, M. Aumailley, J. M. Heard, O. Danos, G. Verdier, and F. L. Cosset. 1994. J. Virol. 68:4609-19.
49. Kim, V. N., K. Mitrophanous, S. M. Kingsman, and K. A. J. 1998. J Virol 72: 811-816.
50. Soneoka, Y., P. M. Cannon, E. E. Ramsdale, J. C. Griffiths, G. Romano, S. M. Kingsman, and A. J. Kingsman. 1995. Nucleic Acids Res. 23:628-33.
51. Sagerstrom, C., and H. Sive. 1996. RNA blot analysis, p. 83-104. In P. Krieg (ed.), A laboratory guide to RNA: isolation, analysis and synthesis, vol. 1. Wiley-Liss Inc., New York.
52. Malirn, M. H., J. Hauber, S. Y. Le, J. V. Maizel, and B. R. Cullen. 1989. Nature. 338:254-7.
53. Felber, B. K., M. Hadzopoulou Cladaras, C. Cladaras, T. Copeland, and G. N. Pavlakis. 1989. proc. Natl. Acad. Sci. USA. 86:1495-1499.
54. Hadzopoulou Cladaras, M., B. K. Felber, C. Cladaras, A. Athanassopoulos, A. Tse, and G. N. Pavlakis. 1989. J. Virol. 63:1265-74.
55. Schneider, R., M. Campbell, G. Nasioulas, B. K. Felber, and G. N. Pavlakis. 1997. J. Virol. 71: 4892-903.
56. Schwartz, S., B. K. Felber, and G. N. Pavlakis. 1992. J. Virol. 66:150-159.
57. Maldarelli, F., M. A. Martin, and K. Strebel. 1991 J. Virol. 65:5732-5743.
58. Mikaelian, I., M. Krieg, M. Gait, and J. Kam. 1996. J. Mol. Biol. 257:246-264.
59. Xu, N., C.-Y. Chen, and A.-B. Shyu. 1997. Mol. Cell. Biol. 17:4611-4621.
60. Naldini, L. 1998. Curr. Opin. Biotechnol. 9:457-463.
61. parolin, C., T. Dorfman, G. Palu, H. Gottlinger, and J. Sodroski. 1994J. Virol.
62. Dull, T., R. Zufferey, M. Kelly, R. Mandel, M. Nguyen, D. Trono, and L. Naldini. 1998. J. Virol. 72:8463-8471.
63. Cui, Y., T. Iwakama, and L.-J. Chang. 1999. J. Virol. 73:6171-6176.
64. Chang, L.-J., V. Urlacher, T. Iwakama, Y. Cui, and J. Zucali. 1999. Gene Ther. 6:715-728.
65. Harrison, G., G. Miele, E. Hunter, and A. Lever. 1998. J. Virol. 72:5886-5896.
66. McBride, M. S., and A. T. Panganiban. 1997. J. Virol. 71:2050-8.
67. Lever, A., H. Gottlinger, W. Haseltine, and J. Sodroski. 1989 J. Virol. 63:4085-7.
68. Brighty, D., and M. Rosenberg. 1994. proc. Natl. Acad. Sci. USA. 91:8314-8318.
69. Arai, T., M. Takada, M. Ui, and H. Iba. 1999. Virology. 260:109-115.
70. Fomerod, M., M. Ohno, M. Yoshida, and 1. W. Mattaj. 1997. Cell. 90:1051-1060.
71. Fridell, R. A., H. P. Bogerd, and B. R. Cullen. 1996. proc. Natl. Acad. Sci. USA. 93:4421-4.
72. pollard, V., and M. Malim. 1998. Annu. Rev. Microbiol. 52:491-532.
73. Stade, K., C. S. Ford, C. Guthrie, and K. Weis. 1997. Cell. 90:1041-1050.
74. Ullman, K. S., M. Powers, A, and D. J. Forbes. 1997. Cell. 90:967-970.
75. Otero, G. C., M. E. Harris, J. E. Donello, and T. J. Hope. 1998. J. Virol. 72:7593-7597.
76. Wolff et al. 1997. Chem Biol. 4:139-147.

1-37. (canceled).

38. A method for producing a replication defective retrovirus, which comprises transfecting a producer cell with the following:
(i) a retroviral genome;
(ii) a nucleotide sequence coding for retroviral gag and pol proteins that is codon optimized for expression in the producer cell; and
(iii) nucleotide sequences encoding other essential viral packaging components not encoded by the nucleotide sequence of (ii), whereby the transfected producer cell produces the replication defective retrovirus.

39. The method of claim 1, wherein the retroviral genome further comprises a nucleotide sequence of interest (NOI).

40. The method of claim 1, wherein the retroviral particle is a lentiviral particle.

41. The method of claim 3, wherein the retroviral particle is substantially derived from HIV-1.

42. The method of claim 4, wherein the codon optimized nucleotide sequence has the sequence shown in SEQ ID NO:15.

43. The method of claim 3, wherein the retroviral particle is substantially derived from EIAV.

44. The method of claim 6, wherein the codon optimized nucleotide sequence has the sequence shown in SEQ ID NO:16.

45. The method of claim 1, wherein the nucleotide sequences of (iii) comprise a nucleotide sequence encoding an env protein.

46. The method of claim 1, wherein one or more of the nucleotide sequences of (i), (ii), and (iii) contain one or more functional accessory genes.

47. The method of claim 1, wherein the nucleotide sequence (i), (ii), and (iii) are devoid of any functional accessory genes.

48. A method for preventing packaging of a retroviral genome in a target cell which comprises the steps of:
(a) transfecting a producer cell with the following to produce retroviral particles:
(i) a retroviral genome;
(ii) a nucleotide sequence coding for retroviral gag and pol proteins that is codon optimized for expression in the producer cell; and
(iii) nucleotide sequences encoding other essential viral packaging components not encoded by one or more of the nucleotide sequences of (ii); and
(b) transfecting a target cell with the retroviral particles of step (a), whereby the retroviral genome in the retroviral particles are prevented from packaging in the target cell.

49. A method for preventing recombination between a retroviral vector genome and a nucleotide sequence encoding a viral polypeptide required for the assembly of the viral genome into retroviral particles, which comprises transfecting a producer cell with the following:
(i) a retroviral genome comprising at least part of a gag nucleotide sequence;
(ii) a nucleotide sequence coding for retroviral gag and pol proteins that is codon optimized for expression in the producer cell; and
(iii) nucleotide sequences encoding other essential viral packaging components not encoded by the nucleotide sequence of (ii).

50. A viral vector system comprising:
(i) a nucleotide sequence of interest; and
(ii) a nucleotide sequence encoding retroviral gag and pot proteins that is codon optimized for expression in the producer cell.

51. The system of claim 13, wherein the viral vector is a retroviral vector.

52. The system of claim 14, wherein the retroviral vector is a lentiviral vector.

53. The system of claim 15, wherein the lentiviral vector is substantially derived from HIV-1 or EIAV.

54. The system of claim 13, wherein the nucleotide sequences of (i) and (ii) also include a nucleotide sequence encoding an envelope protein.

55. The system of claim 17, wherein the nucleotide sequence encoding the envelope protein is codon optimized.

56. The system of claim 13, wherein the nucleotide of interest is selected from the group consisting of a therapeutic gene, a marker gene and a selection gene.

57. The system of claim 13 which comprises one or more functional accessory genes.

58. The system of claim 13 which is devoid of any functional accessory genes.

59. The system of claim 13, wherein the nucleotide sequence of (ii) has the sequence of SEQ ID NO:15 or SEQ ID NO:16.

60. A viral production system comprising:
(i) a viral genome comprising at least one nucleotide sequence of interest; and
(ii) a nucleotide sequence encoding a viral polypeptide required for the assembly of the viral genome into viral particles, wherein the nucleotide sequence encodes retroviral gal and pol proteins that is codon optimized for expression in the producer cell.

61. A method for producing a viral particle, which comprises introducing into a producer cell:
(i) a viral genome comprising at least one nucleotide sequence of interest;
(ii) a nucleotide sequence encoding a viral polypeptide required for the assembly of the viral genome into viral particles, wherein the nucleotide sequence encodes retroviral gag and pol proteins that is codon optimized for expression in the producer cell.
(iii) nucleotide sequences encoding other essential viral packaging components not encoded by one or more of the nucleotide sequences of (ii).

62. A viral particle produced by the method of claim 24.

63. A pharmaceutical composition comprising the viral system of claim 13 together with a pharmaceutically acceptable carrier or diluent.

64. A pharmaceutical composition comprising the viral particle of claim 24 together with a pharmaceutically acceptable carrier or diluent.

65. An isolated nucleic acid comprising the nucleotide sequence of SEQ ID NO:15.

66. An isolated nucleic acid comprising the nucleotide sequence of SEQ ID NO:16.


SEQUENCE LISTING (37 sequences)

SEQ ID NO: 1 - DNA, 4307 bp, Human immunodeficiency virus
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatactgacaatggcagcaatttcaccggtgctacggttagggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaattcactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattag

SEQ ID NO: 2 - DNA, 9772 bp, Artificial Sequence
  Description of Artificial Sequence pSYNGP
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcgccaccatgggcgcccgcgccagcgtgctgtcgggcggcgagctggaccgctgggagaagatccgcctgcgccccggcggcaaaaagaagtacaagctgaagcacatcgtgtgggccagccgcgaactggagcgcttcgccgtgaaccccgggctcctggagaccagcgaggggtgccgccagatcctcggccaactgcagcccagcctgcaaaccggcagcgaggagctgcgcagcctgtacaacaccgtggccacgctgtactgcgtccaccagcgcatcgaaatcaaggatacgaaagaggccctggataaaatcgaagaggaacagaataagagcaaaaagaaggcccaacaggccgccgcggacaccggacacagcaaccaggtcagccagaactaccccatcgtgcagaacatccaggggcagatggtgcaccaggccatctccccccgcacgctgaacgcctgggtgaaggtggtggaagagaaggcttttagcccggaggtgatacccatgttctcagccctgtcagagggagccaccccccaagatctgaacaccatgctcaacacagtggggggacaccaggccgccatgcagatgctgaaggagaccatcaatgaggaggctgccgaatgggatcgtgtgcatccggtgcacgcagggcccatcgcaccgggccagatgcgtgagccacggggctcagacatcgccggaacgactagtacccttcaggaacagatcggctggatgaccaacaacccacccatcccggtgggagaaatctacaaacgctggatcatcctgggcctgaacaagatcgtgcgcatgtatagccctaccagcatcctggacatccgccaaggcccgaaggaaccctttcgcgactacgtggaccggttctacaaaacgctccgcgccgagcaggctagccaggaggtgaagaactggatgaccgaaaccctgctggtccagaacgcgaacccggactgcaagacgatcctgaaggccctgggcccagcggctaccctagaggaaatgatgaccgcctgtcagggagtgggcggacccggccacaaggcacgcgtcctggctgaggccatgagccaggtgaccaactccgctaccatcatgatgcagcgcggcaactttcggaaccaacgcaagatcgtcaagtgcttcaactgtggcaaagaagggcacacagcccgcaactgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcagctcaaggaggctctcctggacaccggagcagacgacaccgtgctggaggagatgtcgttgccaggccgctggaagccgaagatgatcgggggaatcggcggtttcatcaaggtgcgccagtatgaccagatcctcatcgaaatctgcggccacaaggctatcggtaccgtgctggtgggccccacacccgtcaacatcatcggacgcaacctgttgacgcagatcggttgcacgctgaacttccccattagccctatcgagacggtaccggtgaagctgaagcccgggatggacggcccgaaggtcaagcaatggccattgacagaggagaagatcaaggcactggtggagatttgcacagagatggaaaaggaagggaaaatctccaagattgggcctgagaacccgtacaacacgccggtgttcgcaatcaagaagaaggactcgacgaaatggcgcaagctggtggacttccgcgagctgaacaagcgcacgcaagacttctgggaggttcagctgggcatcccgcaccccgcagggctgaagaagaagaaatccgtgaccgtactggatgtgggtgatgcctacttctccgttcccctggacgaagacttcaggaagtacactgccttcacaatcccttcgatcaacaacgagacaccggggattcgatatcagtacaacgtgctgccccagggctggaaaggctctcccgcaatcttccagagtagcatgaccaaaatcctggagcctttccgcaaacagaaccccgacatcgtcatctatcagtacatggatgacttgtacgtgggctctgatctagagatagggcagcaccgcaccaagatcgaggagctgcgccagcacctgttgaggtggggactgaccacacccgacaagaagcaccagaaggagcctcccttcctctggatgggttacgagctgcaccctgacaaatggaccgtgcagcctatcgtgctgccagagaaagacagctggactgtcaacgacatacagaagctggtggggaagttgaactgggccagtcagatttacccagggattaaggtgaggcagctgtgcaaactcctccgcggaaccaaggcactcacagaggtgatccccctaaccgaggaggccgagctcgaactggcagaaaaccgagagatcctaaaggagcccgtgcacggcgtgtactatgacccctccaaggacctgatcgccgagatccagaagcaggggcaaggccagtggacctatcagatttaccaggagcccttcaagaacctgaagaccggcaagtacgcccggatgaggggtgcccacactaacgacgtcaagcagctgaccgaggccgtgcagaagatcaccaccgaaagcatcgtgatctggggaaagactcctaagttcaagctgcccatccagaaggaaacctgggaaacctggtggacagagtattggcaggccacctggattcctgagtgggagttcgtcaacacccctcccctggtgaagctgtggtaccagctggagaaggagcccatagtgggcgccgaaaccttctacgtggatggggccgctaacagggagactaagctgggcaaagccggatacgtcactaaccggggcagacagaaggttgtcaccctcactgacaccaccaaccagaagactgagctgcaggccatttacctcgctttgcaggactcgggcctggaggtgaacatcgtgacagactctcagtatgccctgggcatcattcaagcccagccagaccagagtgagtccgagctggtcaatcagatcatcgagcagctgatcaagaaggaaaaggtctatctggcctgggtacccgcccacaaaggcattggcggcaatgagcaggtcgacaagctggtctcggctggcatcaggaaggtgctattcctggatggcatcgacaaggcccaggacgagcacgagaaataccacagcaactggcgggccatggctagcgacttcaacctgccccctgtggtggccaaagagatcgtggccagctgtgacaagtgtcagctcaagggcgaagccatgcatggccaggtggactgtagccccggcatctggcaactcgattgcacccatctggagggcaaggttatcctggtagccgtccatgtggccagtggctacatcgaggccgaggtcattcccgccgaaacagggcaggagacagcctacttcctcctgaagctggcaggccggtggccagtgaagaccatccatactgacaatggcagcaatttcaccagtgctacggttaaggccgcctgctggtgggcgggaatcaagcaggagttcgggatcccctacaatccccagagtcagggcgtcgtcgagtctatgaataaggagttaaagaagattatcggccaggtcagagatcaggctgagcatctcaagaccgcggtccaaatggcggtattcatccacaatttcaagcggaagggggggattggggggtacagtgcgggggagcggatcgtggacatcatcgcgaccgacatccagactaaggagctgcaaaagcagattaccaagattcagaatttccgggtctactacagggacagcagaaatcccctctggaaaggcccagcgaagctcctctggaagggtgagggggcagtagtgatccaggataatagcgacatcaaggtggtgcccagaagaaaggcgaagatcattagggattatggcaaacagatggcgggtgatgattgcgtggcgagcagacaggatgaggattaggaattgggctagagcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 3 - DNA, 2571 bp, Human immunodeficiency virus
atgagagtgaaggggatcaggaggaattatcagcactggtggggatggggcacgatgctccttgggttattaatgatctgtagtgctacagaaaaattgtgggtcacagtctattatggggtacctgtgtggaaagaagcaaccaccactctattttgtgcatcagatgctaaagcatatgatacagaggtacataatgtttgggccacacaagcctgtgtacccacagaccccaacccacaagaagtagaattggtaaatgtgacagaaaattttaacatgtggaaaaataacatggtagaacagatgcatgaggatataatcagtttatgggatcaaagcctaaagccatgtgtaaaattaaccccactctgtgttactttaaattgcactgatttgaggaatactactaataccaataatagtactgctaataacaatagtaatagcgagggaacaataaagggaggagaaatgaaaaactgctctttcaatatcaccacaagcataagagataagatgcagaaagaatatgcacttctttataaacttgatatagtatcaatagataatgatagtaccagctataggttgataagttgtaatacctcagtcattacacaagcttgtccaaagatatcctttgagccaattcccatacactattgtgccccggctggttttgcgattctaaaatgtaacgataaaaagttcagtggaaaaggatcatgtaaaaatgtcagcacagtacaatgtacacatggaattaggccagtagtatcaactcaactgctgttaaatggcagtctagcagaagaagaggtagtaattagatctgagaatttcactgataatgctaaaaccatcatagtacatctgaatgaatctgtacaaattaattgtacaagacccaactacaataaaagaaaaaggatacatataggaccagggagagcattttatacaacaaaaaatataataggaactataagacaagcacattgtaacattagtagagcaaaatggaatgacactttaagacagatagttagcaaattaaaagaacaatttaagaataaaacaatagtctttaatcaatcctcaggaggggacccagaaattgtaatgcacagttttaattgtggaggggaatttttctactgtaatacatcaccactgtttaatagtacttggaatggtaataatacttggaataatactacagggtcaaataacaatatcacacttcaatgcaaaataaaacaaattataaacatgtggcaggaagtaggaaaagcaatgtatgcccctcccattgaaggacaaattagatgttcatcaaatattacagggctactattaacaagagatggtggtaaggacacggacacgaacgacaccgagatcttcagacctggaggaggagatatgagggacaattggagaagtgaattatataaatataaagtagtaacaattgaaccattaggagtagcacccaccaaggcaaagagaagagtggtgcagagagaaaaaagagcagcgataggagctctgttccttgggttcttaggagcagcaggaagcactatgggcgcagcgtcagtgacgctgacggtacaggccagactattattgtctggtatagtgcaacagcagaacaatttgctgagggccattgaggcgcaacagcatatgttgcaactcacagtctggggcatcaagcagctccaggcaagagtcctggctgtggaaagatacctaaaggatcaacagctcctggggttttggggttgctctggaaaactcatttgcaccactactgtgccttggaatgctagttggagtaataaatctctggatgatatttggaataacatgacctggatgcagtgggaaagagaaattgacaattacacaagcttaatatactcattactagaaaaatcgcaaacccaacaagaaaagaatgaacaagaattattggaattggataaatgggcaagtttgtggaattggtttgacataacaaattggctgtggtatataaaaatattcataatgatagtaggaggcttggtaggtttaagaatagtttttgctgtactttctatagtgaatagagttaggcagggatactcaccattgtcgttgcagacccgccccccagttccgaggggacccgacaggcccgaaggaatcgaagaagaaggtggagagagagacagagacacatccggtcgattagtgcatggattcttagcaattatctgggtcgacctgcggagcctgttcctcttcagctaccaccacagagacttactcttgattgcagcgaggattgtggaacttctgggacgcagggggtgggaagtcctcaaatattggtggaatctcctacagtattggagtcaggaactaaagagtagtgctgttagcttgcttaatgccacagctatagcagtagctgaggggacagatagggttatagaagtactgcaaagagctggtagagctattctccacatacctacaagaataagacagggcttggaaagggctttgctataa

SEQ ID NO: 4 - DNA, 2571 bp, Artificial Sequence
  Description of Artificial Sequence SYNgp-160mn - codon optimised env sequence
atgagggtgaaggggatccgccgcaactaccagcactggtggggctggggcacgatgctcctggggctgctgatgatctgcagcgccaccgagaagctgtgggtgaccgtgtactacggcgtgcccgtgtggaaggaggccaccaccaccctgttctgcgccagcgacgccaaggcgtacgacaccgaggtgcacaacgtgtgggccacccaggcgtgcgtgcccaccgaccccaacccccaggaggtggagctcgtgaacgtgaccgagaacttcaacatgtggaagaacaacatggtggagcagatgcatgaggacatcatcagcctgtgggaccagagcctgaagccctgcgtgaagctgacccccctgtgcgtgaccctgaactgcaccgacctgaggaacaccaccaacaccaacaacagcaccgccaacaacaacagcaacagcgagggcaccatcaagggcggcgagatgaagaactgcagcttcaacatcaccaccagcatccgcgacaagatgcagaaggagtacgccctgctgtacaagctggatatcgtgagcatcgacaacgacagcaccagctaccgcctgatctcctgcaacaccagcgtgatcacccaggcctgccccaagatcagcttcgagcccatccccatccactactgcgcccccgccggcttcgccatcctgaagtgcaacgacaagaagttcagcggcaagggcagctgcaagaacgtgagcaccgtgcagtgcacccacggcatccggccggtggtgagcacccagctcctgctgaacggcagcctggccgaggaggaggtggtgatccgcagcgagaacttcaccgacaacgccaagaccatcatcgtgcacctgaatgagagcgtgcagatcaactgcacgcgtcccaactacaacaagcgcaagcgcatccacatcggccccgggcgcgccttctacaccaccaagaacatcatcggcaccatccgccaggcccactgcaacatctctagagccaagtggaacgacaccctgcgccagatcgtgagcaagctgaaggagcagttcaagaacaagaccatcgtgttcaaccagagcagcggcggcgaccccgagatcgtgatgcacagcttcaactgcggcggcgaattcttctactgcaacaccagccccctgttcaacagcacctggaacggcaacaacacctggaacaacaccaccggcagcaacaacaatattaccctccagtgcaagatcaagcagatcatcaacatgtggcaggaggtgggcaaggccatgtacgccccccccatcgagggccagatccggtgcagcagcaacatcaccggtctgctgctgacccgcgacggcggcaaggacaccgacaccaacgacaccgaaatcttccgccccggcggcggcgacatgcgcgacaactggagatctgagctgtacaagtacaaggtggtgacgatcgagcccctgggcgtggcccccaccaaggccaagcgccgcgtggtgcagcgcgagaagcgggccgccatcggcgccctgttcctgggcttcctgggggcggcgggcagcaccatgggggccgccagcgtgaccctgaccgtgcaggcccgcctgctcctgagcggcatcgtgcagcagcagaacaacctcctccgcgccatcgaggcccagcagcatatgctccagctcaccgtgtggggcatcaagcagctccaggcccgcgtgctggccgtggagcgctacctgaaggaccagcagctcctgggcttctggggctgctccggcaagctgatctgcaccaccacggtaccctggaacgcctcctggagcaacaagagcctggacgacatctggaacaacatgacctggatgcagtgggagcgcgagatcgataactacaccagcctgatctacagcctgctggagaagagccagacccagcaggagaagaacgagcaggagctgctggagctggacaagtgggcgagcctgtggaactggttcgacatcaccaactggctgtggtacatcaaaatcttcatcatgattgtgggcggcctggtgggcctccgcatcgtgttcgccgtgctgagcatcgtgaaccgcgtgcgccagggctacagccccctgagcctccagacccggccccccgtgccgcgcgggcccgaccgccccgagggcatcgaggaggagggcggcgagcgcgaccgcgacaccagcggcaggctcgtgcacggcttcctggcgatcatctgggtcgacctccgcagcctgttcctgttcagctaccaccaccgcgacctgctgctgatcgccgcccgcatcgtggaactcctaggccgccgcggctgggaggtgctgaagtactggtggaacctcctccagtattggagccaggagctgaagtccagcgccgtgagcctgctgaacgccaccgccatcgccgtggccgagggcaccgaccgcgtgatcgaggtgctccagagggccgggagggcgatcctgcacatccccacccgcatccgccaggggctcgagagggcgctgctgtaa

SEQ ID NO: 5 - DNA, 10112 bp, Artificial Sequence
  Description of Artificial Sequence pESYNGP
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagagaattcgccaccatgggcgatcccctcacctggtccaaagccctgaagaaactggaaaaagtcaccgttcagggtagccaaaagcttaccacaggcaattgcaactgggcattgtccctggtggatcttttccacgacactaatttcgttaaggagaaagattggcaactcagagacgtgatccccctcttggaggacgtgacccaaacattgtctgggcaggagcgcgaagctttcgagcgcacctggtgggccatcagcgcagtcaaaatggggctgcaaatcaacaacgtggttgacggtaaagctagctttcaactgctccgcgctaagtacgagaagaaaaccgccaacaagaaacaatccgaacctagcgaggagtacccaattatgatcgacggcgccggcaataggaacttccgcccactgactcccaggggctataccacctgggtcaacaccatccagacaaacggacttttgaacgaagcctcccagaacctgttcggcatcctgtctgtggactgcacctccgaagaaatgaatgcttttctcgacgtggtgccaggacaggctggacagaaacagatcctgctcgatgccattgacaagatcgccgacgactgggataatcgccaccccctgccaaacgcccctctggtggctcccccacaggggcctatccctatgaccgctaggttcattaggggactgggggtgccccgcgaacgccagatggagccagcatttgaccaatttaggcagacctacagacagtggatcatcgaagccatgagcgaggggattaaagtcatgatcggaaagcccaaggcacagaacatcaggcagggggccaaggaaccataccctgagtttgtcgacaggcttctgtcccagattaaatccgaaggccaccctcaggagatctccaagttcttgacagacacactgactatccaaaatgcaaatgaagagtgcagaaacgccatgaggcacctcagacctgaagataccctggaggagaaaatgtacgcatgtcgcgacattggcactaccaagcaaaagatgatgctgctcgccaaggctctgcaaaccggcctggctggtccattcaaaggaggagcactgaagggaggtccattgaaagctgcacaaacatgttataattgtgggaagccaggacatttatctagtcaatgtagagcacctaaagtctgttttaaatgtaaacagcctggacatttctcaaagcaatgcagaagtgttccaaaaaacgggaagcaaggggctcaagggaggccccagaaacaaactttcccgatacaacagaagagtcagcacaacaaatctgttgtacaagagactcctcagactcaaaatctgtacccagatctgagcgaaataaaaaaggaatacaatgtcaaggagaaggatcaagtagaggatctcaacctggacagtttgtgggagtaacatacaatctcgagaagaggcccactaccatcgtcctgatcaatgacacccctcttaatgtgctgctggacaccggagccgacaccagcgttctcactactgctcactataacagactgaaatacagaggaaggaaataccagggcacaggcatcatcggcgttggaggcaacgtcgaaaccttttccactcctgtcaccatcaaaaagaaggggagacacattaaaaccagaatgctggtcgccgacatccccgtcaccatccttggcagagacattctccaggacctgggcgctaaactcgtgctggcacaactgtctaaggaaatcaagttccgcaagatcgagctgaaagagggcacaatgggtccaaaaatcccccagtggcccctgaccaaagagaagcttgagggcgctaaggaaatcgtgcagcgcctgctttctgagggcaagattagcgaggccagcgacaataacccttacaacagccccatctttgtgattaagaaaaggagcggcaaatggagactcctgcaggacctgagggaactcaacaagaccgtccaggtcggaactgagatctctcgcggactgcctcaccccggcggcctgattaaatgcaagcacatgacagtccttgacattggagacgcttattttaccatccccctcgatcctgaatttcgcccctatactgcttttaccatccccagcatcaatcaccaggagcccgataaacgctatgtgtggaagtgcctcccccagggatttgtgcttagcccctacatttaccagaagacacttcaagagatcctccaacctttccgcgaaagatacccagaggttcaactctaccaatatatggacgacctgttcatggggtccaacgggtctaagaagcagcacaaggaactcatcatcgaactgagggcaatcctcctggagaaaggcttcgagacacccgacgacaagctgcaagaagttcctccatatagctggctgggctaccagctttgccctgaaaactggaaagtccagaagatgcagttggatatggtcaagaacccaacactgaacgacgtccagaagctcatgggcaatattacctggatgagctccggaatccctgggcttaccgttaagcacattgccgcaactacaaaaggatgcctggagttgaaccagaaggtcatttggacagaggaagctcagaaggaactggaggagaataatgaaaagattaagaatgctcaagggctccaatactacaatcccgaagaagaaatgttgtgcgaggtcgaaatcactaagaactacgaagccacctatgtcatcaaacagtcccaaggcatcttgtgggccggaaagaaaatcatgaaggccaacaaaggctggtccaccgttaaaaatctgatgctcctgctccagcacgtcgccaccgagtctatcacccgcgtcggcaagtgccccaccttcaaagttcccttcactaaggagcaggtgatgtgggagatgcaaaaaggctggtactactcttggcttcccgagatcgtctacacccaccaagtggtgcacgacgactggagaatgaagcttgtcgaggagcccactagcggaattacaatctataccgacggcggaaagcaaaacggagagggaatcgctgcatacgtcacatctaacggccgcaccaagcaaaagaggctcggccctgtcactcaccaggtggctgagaggatggctatccagatggcccttgaggacactagagacaagcaggtgaacattgtgactgacagctactactgctggaaaaacatcacagagggccttggcctggagggaccccagtctccctggtggcctatcatccagaatatccgcgaaaaggaaattgtctatttcgcctgggtgcctggacacaaaggaatttacggcaaccaactcgccgatgaagccgccaaaattaaagaggaaatcatgcttgcctaccagggcacacagattaaggagaagagagacgaggacgctggctttgacctgtgtgtgccatacgacatcatgattcccgttagcgacacaaagatcattccaaccgatgtcaagatccaggtgccacccaattcatttggttgggtgaccggaaagtccagcatggctaagcagggtcttctgattaacgggggaatcattgatgaaggatacaccggcgaaatccaggtgatctgcacaaatatcggcaaaagcaatattaagcttatcgaagggcagaagttcgctcaactcatcatcctccagcaccacagcaattcaagacaaccttgggacgaaaacaagattagccagagaggtgacaagggcttcggcagcacaggtgtgttctgggtggagaacatccaggaagcacaggacgagcacgagaattggcacacctcccctaagattttggcccgcaattacaagatcccactgactgtggctaagcagatcacacaggaatgcccccactgcaccaaacaaggttctggccccgccggctgcgtgatgaggtcccccaatcactggcaggcagattgcacccacctcgacaacaaaattatcctgaccttcgtggagagcaattccggctacatccacgcaacactcctctccaaggaaaatgcattgtgcacctccctcgcaattctggaatgggccaggctgttctctccaaaatccctgcacaccgacaacggcaccaactttgtggctgaacctgtggtgaatctgctgaagttcctgaaaatcgcccacaccactggcattccctatcaccctgaaagccagggcattgtcgagagggccaacagaactctgaaagaaaagatccaatctcacagagacaatacacagacattggaggccgcacttcagctcgcccttatcacctgcaacaaaggaagagaaagcatgggcggccagaccccctgggaggtcttcatcactaaccaggcccaggtcatccatgaaaagctgctcttgcagcaggcccagtcctccaaaaagttctgcttttataagatccccggtgagcacgactggaaaggtcctacaagagttttgtggaaaggagacggcgcagttgtggtgaacgatgagggcaaggggatcatcgctgtgcccctgacacgcaccaagcttctcatcaagccaaactgaacccggggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 6 - DNA, 10227 bp, Artificial Sequence
  Description of Artificial Sequence LpESYNGP
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagagaattcgagaggggcgcagaccctacctgttgaacctggctgatcgtaggatccccgggacagcagaggagaacttacagaagtcttctggaggtgttcctggccagaacacaggaggacaggtaagatgggcgatcccctcacctggtccaaagccctgaagaaactggaaaaagtcaccgttcagggtagccaaaagcttaccacaggcaattgcaactgggcattgtccctggtggatcttttccacgacactaatttcgttaaggagaaagattggcaactcagagacgtgatccccctcttggaggacgtgacccaaacattgtctgggcaggagcgcgaagctttcgagcgcacctggtgggccatcagcgcagtcaaaatggggctgcaaatcaacaacgtggttgacggtaaagctagctttcaactgctccgcgctaagtacgagaagaaaaccgccaacaagaaacaatccgaacctagcgaggagtacccaattatgatcgacggcgccggcaataggaacttccgcccactgactcccaggggctataccacctgggtcaacaccatccagacaaacggacttttgaacgaagcctcccagaacctgttcggcatcctgtctgtggactgcacctccgaagaaatgaatgcttttctcgacgtggtgccaggacaggctggacagaaacagatcctgctcgatgccattgacaagatcgccgacgactgggataatcgccaccccctgccaaacgcccctctggtggctcccccacaggggcctatccctatgaccgctaggttcattaggggactgggggtgccccgcgaacgccagatggagccagcatttgaccaatttaggcagacctacagacagtggatcatcgaagccatgagcgaggggattaaagtcatgatcggaaagcccaaggcacagaacatcaggcagggggccaaggaaccataccctgagtttgtcgacaggcttctgtcccagattaaatccgaaggccaccctcaggagatctccaagttcttgacagacacactgactatccaaaatgcaaatgaagagtgcagaaacgccatgaggcacctcagacctgaagataccctggaggagaaaatgtacgcatgtcgcgacattggcactaccaagcaaaagatgatgctgctcgccaaggctctgcaaaccggcctggctggtccattcaaaggaggagcactgaagggaggtccattgaaagctgcacaaacatgttataattgtgggaagccaggacatttatctagtcaatgtagagcacctaaagtctgttttaaatgtaaacagcctggacatttctcaaagcaatgcagaagtgttccaaaaaacgggaagcaaggggctcaagggaggccccagaaacaaactttcccgatacaacagaagagtcagcacaacaaatctgttgtacaagagactcctcagactcaaaatctgtacccagatctgagcgaaataaaaaaggaatacaatgtcaaggagaaggatcaagtagaggatctcaacctggacagtttgtgggagtaacatacaatctcgagaagaggcccactaccatcgtcctgatcaatgacacccctcttaatgtgctgctggacaccggagccgacaccagcgttctcactactgctcactataacagactgaaatacagaggaaggaaataccagggcacaggcatcatcggcgttggaggcaacgtcgaaaccttttccactcctgtcaccatcaaaaagaaggggagacacattaaaaccagaatgctggtcgccgacatccccgtcaccatccttggcagagacattctccaggacctgggcgctaaactcgtgctggcacaactgtctaaggaaatcaagttccgcaagatcgagctgaaagagggcacaatgggtccaaaaatcccccagtggcccctgaccaaagagaagcttgagggcgctaaggaaatcgtgcagcgcctgctttctgagggcaagattagcgaggccagcgacaataacccttacaacagccccatctttgtgattaagaaaaggagcggcaaatggagactcctgcaggacctgagggaactcaacaagaccgtccaggtcggaactgagatctctcgcggactgcctcaccccggcggcctgattaaatgcaagcacatgacagtccttgacattggagacgcttattttaccatccccctcgatcctgaatttcgcccctatactgcttttaccatccccagcatcaatcaccaggagcccgataaacgctatgtgtggaagtgcctcccccagggatttgtgcttagcccctacatttaccagaagacacttcaagagatcctccaacctttccgcgaaagatacccagaggttcaactctaccaatatatggacgacctgttcatggggtccaacgggtctaagaagcagcacaaggaactcatcatcgaactgagggcaatcctcctggagaaaggcttcgagacacccgacgacaagctgcaagaagttcctccatatagctggctgggctaccagctttgccctgaaaactggaaagtccagaagatgcagttggatatggtcaagaacccaacactgaacgacgtccagaagctcatgggcaatattacctggatgagctccggaatccctgggcttaccgttaagcacattgccgcaactacaaaaggatgcctggagttgaaccagaaggtcatttggacagaggaagctcagaaggaactggaggagaataatgaaaagattaagaatgctcaagggctccaatactacaatcccgaagaagaaatgttgtgcgaggtcgaaatcactaagaactacgaagccacctatgtcatcaaacagtcccaaggcatcttgtgggccggaaagaaaatcatgaaggccaacaaaggctggtccaccgttaaaaatctgatgctcctgctccagcacgtcgccaccgagtctatcacccgcgtcggcaagtgccccaccttcaaagttcccttcactaaggagcaggtgatgtgggagatgcaaaaaggctggtactactcttggcttcccgagatcgtctacacccaccaagtggtgcacgacgactggagaatgaagcttgtcgaggagcccactagcggaattacaatctataccgacggcggaaagcaaaacggagagggaatcgctgcatacgtcacatctaacggccgcaccaagcaaaagaggctcggccctgtcactcaccaggtggctgagaggatggctatccagatggcccttgaggacactagagacaagcaggtgaacattgtgactgacagctactactgctggaaaaacatcacagagggccttggcctggagggaccccagtctccctggtggcctatcatccagaatatccgcgaaaaggaaattgtctatttcgcctgggtgcctggacacaaaggaatttacggcaaccaactcgccgatgaagccgccaaaattaaagaggaaatcatgcttgcctaccagggcacacagattaaggagaagagagacgaggacgctggctttgacctgtgtgtgccatacgacatcatgattcccgttagcgacacaaagatcattccaaccgatgtcaagatccaggtgccacccaattcatttggttgggtgaccggaaagtccagcatggctaagcagggtcttctgattaacgggggaatcattgatgaaggatacaccggcgaaatccaggtgatctgcacaaatatcggcaaaagcaatattaagcttatcgaagggcagaagttcgctcaactcatcatcctccagcaccacagcaattcaagacaaccttgggacgaaaacaagattagccagagaggtgacaagggcttcggcagcacaggtgtgttctgggtggagaacatccaggaagcacaggacgagcacgagaattggcacacctcccctaagattttggcccgcaattacaagatcccactgactgtggctaagcagatcacacaggaatgcccccactgcaccaaacaaggttctggccccgccggctgcgtgatgaggtcccccaatcactggcaggcagattgcacccacctcgacaacaaaattatcctgaccttcgtggagagcaattccggctacatccacgcaacactcctctccaaggaaaatgcattgtgcacctccctcgcaattctggaatgggccaggctgttctctccaaaatccctgcacaccgacaacggcaccaactttgtggctgaacctgtggtgaatctgctgaagttcctgaaaatcgcccacaccactggcattccctatcaccctgaaagccagggcattgtcgagagggccaacagaactctgaaagaaaagatccaatctcacagagacaatacacagacattggaggccgcacttcagctcgcccttatcacctgcaacaaaggaagagaaagcatgggcggccagaccccctgggaggtcttcatcactaaccaggcccaggtcatccatgaaaagctgctcttgcagcaggcccagtcctccaaaaagttctgcttttataagatccccggtgagcacgactggaaaggtcctacaagagttttgtggaaaggagacggcgcagttgtggtgaacgatgagggcaaggggatcatcgctgtgcccctgacacgcaccaagcttctcatcaagccaaactgaacccggggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 7 - DNA, 10815 bp, Artificial Sequence
  Description of Artificial Sequence pESYNGPRRE
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagagaattcgccaccatgggcgatcccctcacctggtccaaagccctgaagaaactggaaaaagtcaccgttcagggtagccaaaagcttaccacaggcaattgcaactgggcattgtccctggtggatcttttccacgacactaatttcgttaaggagaaagattggcaactcagagacgtgatccccctcttggaggacgtgacccaaacattgtctgggcaggagcgcgaagctttcgagcgcacctggtgggccatcagcgcagtcaaaatggggctgcaaatcaacaacgtggttgacggtaaagctagctttcaactgctccgcgctaagtacgagaagaaaaccgccaacaagaaacaatccgaacctagcgaggagtacccaattatgatcgacggcgccggcaataggaacttccgcccactgactcccaggggctataccacctgggtcaacaccatccagacaaacggacttttgaacgaagcctcccagaacctgttcggcatcctgtctgtggactgcacctccgaagaaatgaatgcttttctcgacgtggtgccaggacaggctggacagaaacagatcctgctcgatgccattgacaagatcgccgacgactgggataatcgccaccccctgccaaacgcccctctggtggctcccccacaggggcctatccctatgaccgctaggttcattaggggactgggggtgccccgcgaacgccagatggagccagcatttgaccaatttaggcagacctacagacagtggatcatcgaagccatgagcgaggggattaaagtcatgatcggaaagcccaaggcacagaacatcaggcagggggccaaggaaccataccctgagtttgtcgacaggcttctgtcccagattaaatccgaaggccaccctcaggagatctccaagttcttgacagacacactgactatccaaaatgcaaatgaagagtgcagaaacgccatgaggcacctcagacctgaagataccctggaggagaaaatgtacgcatgtcgcgacattggcactaccaagcaaaagatgatgctgctcgccaaggctctgcaaaccggcctggctggtccattcaaaggaggagcactgaagggaggtccattgaaagctgcacaaacatgttataattgtgggaagccaggacatttatctagtcaatgtagagcacctaaagtctgttttaaatgtaaacagcctggacatttctcaaagcaatgcagaagtgttccaaaaaacgggaagcaaggggctcaagggaggccccagaaacaaactttcccgatacaacagaagagtcagcacaacaaatctgttgtacaagagactcctcagactcaaaatctgtacccagatctgagcgaaataaaaaaggaatacaatgtcaaggagaaggatcaagtagaggatctcaacctggacagtttgtgggagtaacatacaatctcgagaagaggcccactaccatcgtcctgatcaatgacacccctcttaatgtgctgctggacaccggagccgacaccagcgttctcactactgctcactataacagactgaaatacagaggaaggaaataccagggcacaggcatcatcggcgttggaggcaacgtcgaaaccttttccactcctgtcaccatcaaaaagaaggggagacacattaaaaccagaatgctggtcgccgacatccccgtcaccatccttggcagagacattctccaggacctgggcgctaaactcgtgctggcacaactgtctaaggaaatcaagttccgcaagatcgagctgaaagagggcacaatgggtccaaaaatcccccagtggcccctgaccaaagagaagcttgagggcgctaaggaaatcgtgcagcgcctgctttctgagggcaagattagcgaggccagcgacaataacccttacaacagccccatctttgtgattaagaaaaggagcggcaaatggagactcctgcaggacctgagggaactcaacaagaccgtccaggtcggaactgagatctctcgcggactgcctcaccccggcggcctgattaaatgcaagcacatgacagtccttgacattggagacgcttattttaccatccccctcgatcctgaatttcgcccctatactgcttttaccatccccagcatcaatcaccaggagcccgataaacgctatgtgtggaagtgcctcccccagggatttgtgcttagcccctacatttaccagaagacacttcaagagatcctccaacctttccgcgaaagatacccagaggttcaactctaccaatatatggacgacctgttcatggggtccaacgggtctaagaagcagcacaaggaactcatcatcgaactgagggcaatcctcctggagaaaggcttcgagacacccgacgacaagctgcaagaagttcctccatatagctggctgggctaccagctttgccctgaaaactggaaagtccagaagatgcagttggatatggtcaagaacccaacactgaacgacgtccagaagctcatgggcaatattacctggatgagctccggaatccctgggcttaccgttaagcacattgccgcaactacaaaaggatgcctggagttgaaccagaaggtcatttggacagaggaagctcagaaggaactggaggagaataatgaaaagattaagaatgctcaagggctccaatactacaatcccgaagaagaaatgttgtgcgaggtcgaaatcactaagaactacgaagccacctatgtcatcaaacagtcccaaggcatcttgtgggccggaaagaaaatcatgaaggccaacaaaggctggtccaccgttaaaaatctgatgctcctgctccagcacgtcgccaccgagtctatcacccgcgtcggcaagtgccccaccttcaaagttcccttcactaaggagcaggtgatgtgggagatgcaaaaaggctggtactactcttggcttcccgagatcgtctacacccaccaagtggtgcacgacgactggagaatgaagcttgtcgaggagcccactagcggaattacaatctataccgacggcggaaagcaaaacggagagggaatcgctgcatacgtcacatctaacggccgcaccaagcaaaagaggctcggccctgtcactcaccaggtggctgagaggatggctatccagatggcccttgaggacactagagacaagcaggtgaacattgtgactgacagctactactgctggaaaaacatcacagagggccttggcctggagggaccccagtctccctggtggcctatcatccagaatatccgcgaaaaggaaattgtctatttcgcctgggtgcctggacacaaaggaatttacggcaaccaactcgccgatgaagccgccaaaattaaagaggaaatcatgcttgcctaccagggcacacagattaaggagaagagagacgaggacgctggctttgacctgtgtgtgccatacgacatcatgattcccgttagcgacacaaagatcattccaaccgatgtcaagatccaggtgccacccaattcatttggttgggtgaccggaaagtccagcatggctaagcagggtcttctgattaacgggggaatcattgatgaaggatacaccggcgaaatccaggtgatctgcacaaatatcggcaaaagcaatattaagcttatcgaagggcagaagttcgctcaactcatcatcctccagcaccacagcaattcaagacaaccttgggacgaaaacaagattagccagagaggtgacaagggcttcggcagcacaggtgtgttctgggtggagaacatccaggaagcacaggacgagcacgagaattggcacacctcccctaagattttggcccgcaattacaagatcccactgactgtggctaagcagatcacacaggaatgcccccactgcaccaaacaaggttctggccccgccggctgcgtgatgaggtcccccaatcactggcaggcagattgcacccacctcgacaacaaaattatcctgaccttcgtggagagcaattccggctacatccacgcaacactcctctccaaggaaaatgcattgtgcacctccctcgcaattctggaatgggccaggctgttctctccaaaatccctgcacaccgacaacggcaccaactttgtggctgaacctgtggtgaatctgctgaagttcctgaaaatcgcccacaccactggcattccctatcaccctgaaagccagggcattgtcgagagggccaacagaactctgaaagaaaagatccaatctcacagagacaatacacagacattggaggccgcacttcagctcgcccttatcacctgcaacaaaggaagagaaagcatgggcggccagaccccctgggaggtcttcatcactaaccaggcccaggtcatccatgaaaagctgctcttgcagcaggcccagtcctccaaaaagttctgcttttataagatccccggtgagcacgactggaaaggtcctacaagagttttgtggaaaggagacggcgcagttgtggtgaacgatgagggcaaggggatcatcgctgtgcccctgacacgcaccaagcttctcatcaagccaaactgaacccgacgaatcccagggggaatctcaacccctattacccaacagtcagaaaaatctaagtgtgaggagaacacaatgtttcaaccttattgttataataatgacagtaagaacagcatggcagaatcgaaggaagcaagagaccaagaaatgaacctgaaagaagaatctaaagaagaaaaaagaagaaatgactggtggaaaataggtatgtttctgttatgcttagccagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgactgttgcattaaagcccaagaaggaactctcgctatcccttgctgtggatttcccttatggctattttgggggtcggggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 8 - DNA, 10930 bp, Artificial Sequence
  Description of Artificial Sequence LpESYNGPRRE
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagagaattcgagaggggcgcagaccctacctgttgaacctggctgatcgtaggatccccgggacagcagaggagaacttacagaagtcttctggaggtgttcctggccagaacacaggaggacaggtaagatgggcgatcccctcacctggtccaaagccctgaagaaactggaaaaagtcaccgttcagggtagccaaaagcttaccacaggcaattgcaactgggcattgtccctggtggatcttttccacgacactaatttcgttaaggagaaagattggcaactcagagacgtgatccccctcttggaggacgtgacccaaacattgtctgggcaggagcgcgaagctttcgagcgcacctggtgggccatcagcgcagtcaaaatggggctgcaaatcaacaacgtggttgacggtaaagctagctttcaactgctccgcgctaagtacgagaagaaaaccgccaacaagaaacaatccgaacctagcgaggagtacccaattatgatcgacggcgccggcaataggaacttccgcccactgactcccaggggctataccacctgggtcaacaccatccagacaaacggacttttgaacgaagcctcccagaacctgttcggcatcctgtctgtggactgcacctccgaagaaatgaatgcttttctcgacgtggtgccaggacaggctggacagaaacagatcctgctcgatgccattgacaagatcgccgacgactgggataatcgccaccccctgccaaacgcccctctggtggctcccccacaggggcctatccctatgaccgctaggttcattaggggactgggggtgccccgcgaacgccagatggagccagcatttgaccaatttaggcagacctacagacagtggatcatcgaagccatgagcgaggggattaaagtcatgatcggaaagcccaaggcacagaacatcaggcagggggccaaggaaccataccctgagtttgtcgacaggcttctgtcccagattaaatccgaaggccaccctcaggagatctccaagttcttgacagacacactgactatccaaaatgcaaatgaagagtgcagaaacgccatgaggcacctcagacctgaagataccctggaggagaaaatgtacgcatgtcgcgacattggcactaccaagcaaaagatgatgctgctcgccaaggctctgcaaaccggcctggctggtccattcaaaggaggagcactgaagggaggtccattgaaagctgcacaaacatgttataattgtgggaagccaggacatttatctagtcaatgtagagcacctaaagtctgttttaaatgtaaacagcctggacatttctcaaagcaatgcagaagtgttccaaaaaacgggaagcaaggggctcaagggaggccccagaaacaaactttcccgatacaacagaagagtcagcacaacaaatctgttgtacaagagactcctcagactcaaaatctgtacccagatctgagcgaaataaaaaaggaatacaatgtcaaggagaaggatcaagtagaggatctcaacctggacagtttgtgggagtaacatacaatctcgagaagaggcccactaccatcgtcctgatcaatgacacccctcttaatgtgctgctggacaccggagccgacaccagcgttctcactactgctcactataacagactgaaatacagaggaaggaaataccagggcacaggcatcatcggcgttggaggcaacgtcgaaaccttttccactcctgtcaccatcaaaaagaaggggagacacattaaaaccagaatgctggtcgccgacatccccgtcaccatccttggcagagacattctccaggacctgggcgctaaactcgtgctggcacaactgtctaaggaaatcaagttccgcaagatcgagctgaaagagggcacaatgggtccaaaaatcccccagtggcccctgaccaaagagaagcttgagggcgctaaggaaatcgtgcagcgcctgctttctgagggcaagattagcgaggccagcgacaataacccttacaacagccccatctttgtgattaagaaaaggagcggcaaatggagactcctgcaggacctgagggaactcaacaagaccgtccaggtcggaactgagatctctcgcggactgcctcaccccggcggcctgattaaatgcaagcacatgacagtccttgacattggagacgcttattttaccatccccctcgatcctgaatttcgcccctatactgcttttaccatccccagcatcaatcaccaggagcccgataaacgctatgtgtggaagtgcctcccccagggatttgtgcttagcccctacatttaccagaagacacttcaagagatcctccaacctttccgcgaaagatacccagaggttcaactctaccaatatatggacgacctgttcatggggtccaacgggtctaagaagcagcacaaggaactcatcatcgaactgagggcaatcctcctggagaaaggcttcgagacacccgacgacaagctgcaagaagttcctccatatagctggctgggctaccagctttgccctgaaaactggaaagtccagaagatgcagttggatatggtcaagaacccaacactgaacgacgtccagaagctcatgggcaatattacctggatgagctccggaatccctgggcttaccgttaagcacattgccgcaactacaaaaggatgcctggagttgaaccagaaggtcatttggacagaggaagctcagaaggaactggaggagaataatgaaaagattaagaatgctcaagggctccaatactacaatcccgaagaagaaatgttgtgcgaggtcgaaatcactaagaactacgaagccacctatgtcatcaaacagtcccaaggcatcttgtgggccggaaagaaaatcatgaaggccaacaaaggctggtccaccgttaaaaatctgatgctcctgctccagcacgtcgccaccgagtctatcacccgcgtcggcaagtgccccaccttcaaagttcccttcactaaggagcaggtgatgtgggagatgcaaaaaggctggtactactcttggcttcccgagatcgtctacacccaccaagtggtgcacgacgactggagaatgaagcttgtcgaggagcccactagcggaattacaatctataccgacggcggaaagcaaaacggagagggaatcgctgcatacgtcacatctaacggccgcaccaagcaaaagaggctcggccctgtcactcaccaggtggctgagaggatggctatccagatggcccttgaggacactagagacaagcaggtgaacattgtgactgacagctactactgctggaaaaacatcacagagggccttggcctggagggaccccagtctccctggtggcctatcatccagaatatccgcgaaaaggaaattgtctatttcgcctgggtgcctggacacaaaggaatttacggcaaccaactcgccgatgaagccgccaaaattaaagaggaaatcatgcttgcctaccagggcacacagattaaggagaagagagacgaggacgctggctttgacctgtgtgtgccatacgacatcatgattcccgttagcgacacaaagatcattccaaccgatgtcaagatccaggtgccacccaattcatttggttgggtgaccggaaagtccagcatggctaagcagggtcttctgattaacgggggaatcattgatgaaggatacaccggcgaaatccaggtgatctgcacaaatatcggcaaaagcaatattaagcttatcgaagggcagaagttcgctcaactcatcatcctccagcaccacagcaattcaagacaaccttgggacgaaaacaagattagccagagaggtgacaagggcttcggcagcacaggtgtgttctgggtggagaacatccaggaagcacaggacgagcacgagaattggcacacctcccctaagattttggcccgcaattacaagatcccactgactgtggctaagcagatcacacaggaatgcccccactgcaccaaacaaggttctggccccgccggctgcgtgatgaggtcccccaatcactggcaggcagattgcacccacctcgacaacaaaattatcctgaccttcgtggagagcaattccggctacatccacgcaacactcctctccaaggaaaatgcattgtgcacctccctcgcaattctggaatgggccaggctgttctctccaaaatccctgcacaccgacaacggcaccaactttgtggctgaacctgtggtgaatctgctgaagttcctgaaaatcgcccacaccactggcattccctatcaccctgaaagccagggcattgtcgagagggccaacagaactctgaaagaaaagatccaatctcacagagacaatacacagacattggaggccgcacttcagctcgcccttatcacctgcaacaaaggaagagaaagcatgggcggccagaccccctgggaggtcttcatcactaaccaggcccaggtcatccatgaaaagctgctcttgcagcaggcccagtcctccaaaaagttctgcttttataagatccccggtgagcacgactggaaaggtcctacaagagttttgtggaaaggagacggcgcagttgtggtgaacgatgagggcaaggggatcatcgctgtgcccctgacacgcaccaagcttctcatcaagccaaactgaacccgacgaatcccagggggaatctcaacccctattacccaacagtcagaaaaatctaagtgtgaggagaacacaatgtttcaaccttattgttataataatgacagtaagaacagcatggcagaatcgaaggaagcaagagaccaagaaatgaacctgaaagaagaatctaaagaagaaaaaagaagaaatgactggtggaaaataggtatgtttctgttatgcttagccagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgactgttgcattaaagcccaagaaggaactctcgctatcccttgctgtggatttcccttatggctattttgggggtcggggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 9 - DNA, 11131 bp, Artificial Sequence
  Description of Artificial Sequence pONY4.0Z
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggcagatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagatgggagaccctttgacatggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatctctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcggccccaagcttcagctgctcgaggatctgcggatccggggaattccccagtctcaggatccaccatgggggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatccgcagatccggctgtggaatgtgtgtcagttagggtgtggaaagtccccaggctccccagcaggcagaagtatgcaaagcatgcctgcaggaattcgatatcaagcttatcgataccgtcgacctcgagggggggcccggtacccagcttttgttccctttagtgagggttaattgcgcgggaagtatttatcactaatcaagcacaagtaatacatgagaaacttttactacagcaagcacaatcctccaaaaaattttgtttttacaaaatccctggtgaacatgattggaagggacctactagggtgctgtggaagggtgatggtgcagtagtagttaatgatgaaggaaagggaataattgctgtaccattaaccaggactaagttactaataaaaccaaattgagtattgttgcaggaagcaagacccaactaccattgtcagctgtgtttcctgaggtctctaggaattgattacctcgatgcttcattaaggaagaagaataaacaaagactgaaggcaatccaacaaggaagacaacctcaatatttgttataaggtttgatatatgggagtatttggtaaaggggtaacatggtcagcatcgcattctatgggggaatcccagggggaatctcaacccctattacccaacagtcagaaaaatctaagtgtgaggagaacacaatgtttcaaccttattgttataataatgacagtaagaacagcatggcagaatcgaaggaagcaagagaccaagaaatgaacctgaaagaagaatctaaagaagaaaaaagaagaaatgactggtggaaaataggtatgtttctgttatgcttagcaggaactactggaggaatactttggtggtatgaaggactcccacagcaacattatatagggttggtggcgatagggggaagattaaacggatctggccaatcaaatgctatagaatgctggggttccttcccggggtgtagaccatttcaaaattacttcagttatgagaccaatagaagcatgcatatggataataatactgctacattattagaagctttaaccaatataactgctctataaataacaaaacagaattagaaacatggaagttagtaaagacttctggcataactcctttacctatttcttctgaagctaacactggactaattagacataagagagattttggtataagtgcaatagtggcagctattgtagccgctactgctattgctgctagcgctactatgtcttatgttgctctaactgaggttaacaaaataatggaagtacaaaatcatacttttgaggtagaaaatagtactctaaatggtatggatttaatagaacgacaaataaagatattatatgctatgattcttcaaacacatgcagatgttcaactgttaaaggaaagacaacaggtagaggagacatttaatttaattggatgtatagaaagaacacatgtattttgtcatactggtcatccctggaatatgtcatggggacatttaaatgagtcaacacaatgggatgactgggtaagcaaaatggaagatttaaatcaagagatactaactacacttcatggagccaggaacaatttggcacaatccatgataacattcaatacaccagatagtatagctcaatttggaaaagacctttggagtcatattggaaattggattcctggattgggagcttccattataaaatatatagtgatgtttttgcttatttatttgttactaacctcttcgcctaagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgactgttgcattaaagcccaagaaggaactctcgctatcccttgctgtggatttcccttatggctattttggggactagtaattatagtaggacgcatagcaggctatggattacgtggactcgctgttataataaggatttgtattagaggcttaaatttgatatttgaaataatcagaaaaatgcttgattatattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgttaacttcctgtttttacagtatataagtgcttgtattctgacaattgggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 10 - DNA, 10998 bp, Artificial Sequence
  Description of Artificial Sequence pONY8.0Z
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatctctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcggccccaagcttcagctgctcgaggatctgcggatccggggaattccccagtctcaggatccaccatgggggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatccgcagatccggctgtggaatgtgtgtcagttagggtgtggaaagtccccaggctccccagcaggcagaagtatgcaaagcatgcctgcaggaattcgatatcaagcttatcgataccgtcgacctcgagggggggcccggtacccagcttttgttccctttagtgagggttaattgcgcgggaagtatttatcactaatcaagcacaagtaatacatgagaaacttttactacagcaagcacaatcctccaaaaaattttgtttttacaaaatccctggtgaacatgattggaagggacctactagggtgctgtggaagggtgatggtgcagtagtagttaatgatgaaggaaagggaataattgctgtaccattaaccaggactaagttactaataaaaccaaattgagtattgttgcaggaagcaagacccaactaccattgtcagctgtgtttcctgacctcaatatttgttataaggtttgatatgaatcccagggggaatctcaacccctattacccaacagtcagaaaaatctaagtgtgaggagaacacaatgtttcaaccttattgttataataatgacagtaagaacagcatggcagaatcgaaggaagcaagagaccaagaatgaacctgaaagaagaatctaaagaagaaaaaagaagaaatgactggtggaaaataggtatgtttctgttatgcttagcaggaactactggaggaatactttggtggtatgaaggactcccacagcaacattatatagggttggtggcgatagggggaagattaaacggatctggccaatcaaatgctatagaatgctggggttccttcccggggtgtagaccatttcaaaattacttcagttatgagaccaatagaagcatgcatatggataataatactgctacattattagaagctttaaccaatataactgctctataaataacaaaacagaattagaaacatggaagttagtaaagacttctggcataactcctttacctatttcttctgaagctaacactggactaattagacataagagagattttggtataagtgcaatagtggcagctattgtagccgctactgctattgctgctagcgctactatgtcttatgttgctctaactgaggttaacaaaataatggaagtacaaaatcatacttttgaggtagaaaatagtactctaaatggtatggatttaatagaacgacaaataaagatattatatgctatgattcttcaaacacatgcagatgttcaactgttaaaggaaagacaacaggtagaggagacatttaatttaattggatgtatagaaagaacacatgtattttgtcatactggtcatccctggaatatgtcatggggacatttaaatgagtcaacacaatgggatgactgggtaagcaaaatggaagatttaaatcaagagatactaactacacttcatggagccaggaacaatttggcacaatccatgataacattcaatacaccagatagtatagctcaatttggaaaagacctttggagtcatattggaaattggattcctggattgggagcttccattataaaatatatagtgatgtttttgcttatttatttgttactaacctcttcgcctaagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgactgttgcattaaagcccaagaaggaactctcgctatcccttgctgtggatttcccttatggctattttggggactagtaattatagtaggacgcatagcaggctatggattacgtggactcgctgttataataaggatttgtattagaggcttaaatttgatatttgaaataatcagaaaaatgcttgattatattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgttaacttcctgtttttacagtatataagtgcttgtattctgacaattgggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

SEQ ID NO: 11 - DNA, 8870 bp, Artificial Sequence
  Description of Artificial Sequence pONY8.1Z
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatctctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcggccccaagcttcagctgctcgaggatctgcggatccggggaattccccagtctcaggatccaccatgggggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatccgcagatccggctgtggaatgtgtgtcagttagggtgtggaaagtccccaggctccccagcaggcagaagtatgcaaagcatgcctgcaggaattcgatatcaagcttatcgataccgtcgaattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgttaacttcctgtttttacagtatataagtgcttgtattctgacaattgggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

SEQ ID NO: 12 - DNA, 12481 bp, Artificial Sequence
  Description of Artificial Sequence pONY3.1
agatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgaggtcgacggtatcgcccgaacagggacctgagaggggcgcagaccctacctgttgaacctggctgatcgtaggatccccgggacagcagaggagaacttacagaagtcttctggaggtgttcctggccagaacacaggaggacaggtaagatgggagaccctttgacatggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatccaatcatgatagatggggctggaaacagaaattttagacctctaacacctagaggatatactacttgggtgaataccatacagacaaatggtctattaaatgaagctagtcaaaacttatttgggatattatcagtagactgtacttctgaagaaatgaatgcatttttggatgtggtacctggccaggcaggacaaaagcagatattacttgatgcaattgataagatagcagatgattgggataatagacatccattaccgaatgctccactggtggcaccaccacaagggcctattcccatgacagcaaggtttattagaggtttaggagtacctagagaaagacagatggagcctgcttttgatcagtttaggcagacatatagacaatggataatagaagccatgtcagaaggcatcaaagtgatgattggaaaacctaaagctcaaaatattaggcaaggagctaaggaaccttacccagaatttgtagacagactattatcccaaataaaaagtgagggacatccacaagagatttcaaaattcttgactgatacactgactattcagaacgcaaatgaggaatgtagaaatgctatgagacatttaagaccagaggatacattagaagagaaaatgtatgcttgcagagacattggaactacaaaacaaaagatgatgttattggcaaaagcacttcagactggtcttgcgggcccatttaaaggtggagccttgaaaggagggccactaaaggcagcacaaacatgttataactgtgggaagccaggacatttatctagtcaatgtagagcacctaaagtctgttttaaatgtaaacagcctggacatttctcaaagcaatgcagaagtgttccaaaaaacgggaagcaaggggctcaagggaggccccagaaacaaactttcccgatacaacagaagagtcagcacaacaaatctgttgtacaagagactcctcagactcaaaatctgtacccagatctgagcgaaataaaaaaggaatacaatgtcaaggagaaggatcaagtagaggatctcaacctggacagtttgtgggagtaacatataatctagagaaaaggcctactacaatagtattaattaatgatactcccttaaatgtactgttagacacaggagcagatacttcagtgttgactactgcacattataataggttaaaatatagagggagaaaatatcaagggacgggaataataggagtgggaggaaatgtggaaacattttctacgcctgtgactataaagaaaaagggtagacacattaagacaagaatgctagtggcagatattccagtgactattttgggacgagatattcttcaggacttaggtgcaaaattggttttggcacagctctccaaggaaataaaatttagaaaaatagagttaaaagagggcacaatggggccaaaaattcctcaatggccactcactaaggagaaactagaaggggccaaagagatagtccaaagactattgtcagagggaaaaatatcagaagctagtgacaataatccttataattcacccatatttgtaataaaaaagaggtctggcaaatggaggttattacaagatctgagagaattaaacaaaacagtacaagtaggaacggaaatatccagaggattgcctcacccgggaggattaattaaatgtaaacacatgactgtattagatattggagatgcatatttcactatacccttagatccagagtttagaccatatacagctttcactattccctccattaatcatcaagaaccagataaaagatatgtgtggaaatgtttaccacaaggattcgtgttgagcccatatatatatcagaaaacattacaggaaattttacaaccttttagggaaagatatcctgaagtacaattgtatcaatatatggatgatttgttcatgggaagtaatggttctaaaaaacaacacaaagagttaatcatagaattaagggcgatcttactggaaaagggttttgagacaccagatgataaattacaagaagtgccaccttatagctggctaggttatcaactttgtcctgaaaattggaaagtacaaaaaatgcaattagacatggtaaagaatccaacccttaatgatgtgcaaaaattaatggggaatataacatggatgagctcagggatcccagggttgacagtaaaacacattgcagctactactaagggatgtttagagttgaatcaaaaagtaatttggacggaagaggcacaaaaagagttagaagaaaataatgagaagattaaaaatgctcaagggttacaatattataatccagaagaagaaatgttatgtgaggttgaaattacaaaaaattatgaggcaacttatgttataaaacaatcacaaggaatcctatgggcaggtaaaaagattatgaaggctaataagggatggtcaacagtaaaaaatttaatgttattgttgcaacatgtggcaacagaaagtattactagagtaggaaaatgtccaacgtttaaggtaccatttaccaaagagcaagtaatgtgggaaatgcaaaaaggatggtattattcttggctcccagaaatagtatatacacatcaagtagttcatgatgattggagaatgaaattggtagaagaacctacatcaggaataacaatatacactgatgggggaaaacaaaatggagaaggaatagcagcttatgtgaccagtaatgggagaactaaacagaaaaggttaggacctgtcactcatcaagttgctgaaagaatggcaatacaaatggcattagaggataccagagataaacaagtaaatatagtaactgatagttattattgttggaaaaatattacagaaggattaggtttagaaggaccacaaagtccttggtggcctataatacaaaatatacgagaaaaagagatagtttattttgcttgggtacctggtcacaaagggatatatggtaatcaattggcagatgaagccgcaaaaataaaagaagaaatcatgctagcataccaaggcacacaaattaaagagaaaagagatgaagatgcagggtttgacttatgtgttccttatgacatcatgatacctgtatctgacacaaaaatcatacccacagatgtaaaaattcaagttcctcctaatagctttggatgggtcactgggaaatcatcaatggcaaaacaggggttattaattaatggaggaataattgatgaaggatatacaggagaaatacaagtgatatgtactaatattggaaaaagtaatattaaattaatagagggacaaaaatttgcacaattaattatactacagcatcactcaaattccagacagccttgggatgaaaataaaatatctcagagaggggataaaggatttggaagtacaggagtattctgggtagaaaatattcaggaagcacaagatgaacatgagaattggcatacatcaccaaagatattggcaagaaattataagataccattgactgtagcaaaacagataactcaagaatgtcctcattgcactaagcaaggatcaggacctgcaggttgtgtcatgagatctcctaatcattggcaggcagattgcacacatttggacaataagataatattgacttttgtagagtcaaattcaggatacatacatgctacattattgtcaaaagaaaatgcattatgtacttcattggctattttagaatgggcaagattgttttcaccaaagtccttacacacagataacggcactaattttgtggcagaaccagttgtaaatttgttgaagttcctaaagatagcacataccacaggaataccatatcatccagaaagtcagggtattgtagaaagggcaaataggaccttgaaagagaagattcaaagtcatagagacaacactcaaacactggaggcagctttacaacttgctctcattacttgtaacaaagggagggaaagtatgggaggacagacaccatgggaagtatttatcactaatcaagcacaagtaatacatgagaaacttttactacagcaagcacaatcctccaaaaaattttgtttttacaaaatccctggtgaacatgattggaagggacctactagggtgctgtggaagggtgatggtgcagtagtagttaatgatgaaggaaagggaataattgctgtaccattaaccaggactaagttactaataaaaccaaattgagtattgttgcaggaagcaagacccaactaccattgtcagctgtgtttcctgaggtctctaggaattgattacctcgatgcttcattaaggaagaagaataaacaaagactgaaggcaatccaacaaggaagacaacctcaatatttgttataaggtttgatatatgggagtatttggtaaaggggtaacatggtcagcatcgcattctatgggggaatcccagggggaatctcaacccctattacccaacagtcagaaaaatctaagtgtgaggagaacacaatgtttcaaccttattgttataataatgacagtaagaacagcatggcagaatcgaaggaagcaagagaccaagaaatgaacctgaaagaagaatctaaagaagaaaaaagaagaaatgactggtggaaaataggtatgtttctgttatgcttagcaggaactactggaggaatactttggtggtatgaaggactcccacagcaacattatatagggttggtggcgatagggggaagattaaacggatctggccaatcaaatgctatagaatgctggggttccttcccggggtgtagaccatttcaaaattacttcagttatgagaccaatagaagcatgcatatggataataatactgctacattattagaagctttaaccaatataactgctctataaataacaaaacagaattagaaacatggaagttagtaaagacttctggcataactcctttacctatttcttctgaagctaacactggactaattagacataagagagattttggtataagtgcaatagtggcagctattgtagccgctactgctattgctgctagcgctactatgtcttatgttgctctaactgaggttaacaaaataatggaagtacaaaatcatacttttgaggtagaaaatagtactctaaatggtatggatttaatagaacgacaaataaagatattatatgctatgattcttcaaacacatgcagatgttcaactgttaaaggaaagacaacaggtagaggagacatttaatttaattggatgtatagaaagaacacatgtattttgtcatactggtcatccctggaatatgtcatggggacatttaaatgagtcaacacaatgggatgactgggtaagcaaaatggaagatttaaatcaagagatactaactacacttcatggagccaggaacaatttggcacaatccatgataacattcaatacaccagatagtatagctcaatttggaaaagacctttggagtcatattggaaattggattcctggattgggagcttccattataaaatatatagtgatgtttttgcttatttatttgttactaacctcttcgcctaagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgactgttgcattaaagcccaagaaggaactctcgctatcccttgctgtggatttcccttatggctattttggggactagtaattatagtaggacgcatagcaggctatggattacgtggactcgctgttataataaggatttgtattagaggcttaaatttgatatttgaaataatcagaaaaatgcttgattatattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgtggtacctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 13 - DNA, 6395 bp, Artificial Sequence
  Description of Artificial Sequence pCIneoERev
tgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagtaacggccgccagtgtgctggaattcggcttatggcagaatcgaaggaagcaagagaccaagaaatgaacctgaaagaagaatctaaagaagaaaaaagaagaaatgactggtggaaaatagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgaagccgaattctgcagatatccatcacactggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 14 - DNA, 5961 bp, Artificial Sequence
  Description of Artificial Sequence pESYNREV
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcgccaccatggctgagagcaaggaggccagggatcaagagatgaacctcaaggaagagagcaaagaggagaagcgccgcaacgactggtggaagatcgacccacaaggccccctggagggggaccagtggtgccgcgtgctgagacagtccctgcccgaggagaagattcctagccagacctgcatcgccagaagacacctcggccccggtcccacccagcacacaccctccagaagggataggtggattaggggccagattttgcaagccgaggtcctccaagaaaggctggaatggagaattaggggcgtgcaacaagccgctaaagagctgggagaggtgaatcgcggcatctggagggagctctacttccgcgaggaccagaggggcgatttctccgcatggggaggctaccagagggcacaagaaaggctgtggggcgagcagagcagcccccgcgtcttgaggcccggagactccaaaagacgccgcaaacacctgtgaagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 15 - DNA, 4307 bp, Artificial Sequence
  Description of Artificial Sequence Codon optimised HIV gag-pol
atgggcgcccgcgccagcgtgctgtcgggcggcgagctggaccgctgggagaagatccgcctgcgccccggcggcaaaaagaagtacaagctgaagcacatcgtgtgggccagccgcgaactggagcgcttcgccgtgaaccccgggctcctggagaccagcgaggggtgccgccagatcctcggccaactgcagcccagcctgcaaaccggcagcgaggagctgcgcagcctgtacaacaccgtggccacgctgtactgcgtccaccagcgcatcgaaatcaaggatacgaaagaggccctggataaaatcgaagaggaacagaataagagcaaaaagaaggcccaacaggccgccgcggacaccggacacagcaaccaggtcagccagaactaccccatcgtgcagaacatccaggggcagatggtgcaccaggccatctccccccgcacgctgaacgcctgggtgaaggtggtggaagagaaggcttttagcccggaggtgatacccatgttctcagccctgtcagagggagccaccccccaagatctgaacaccatgctcaacacagtggggggacaccaggccgccatgcagatgctgaaggagaccatcaatgaggaggctgccgaatgggatcgtgtgcatccggtgcacgcagggcccatcgcaccgggccagatgcgtgagccacggggctcagacatcgccggaacgactagtacccttcaggaacagatcggctggatgaccaacaacccacccatcccggtgggagaaatctacaaacgctggatcatcctgggcctgaacaagatcgtgcgcatgtatagccctaccagcatcctggacatccgccaaggcccgaaggaaccctttcgcgactacgtggaccggttctacaaaacgctccgcgccgagcaggctagccaggaggtgaagaactggatgaccgaaaccctgctggtccagaacgcgaacccggactgcaagacgatcctgaaggccctgggcccagcggctaccctagaggaaatgatgaccgcctgtcagggagtgggcggacccggccacaaggcacgcgtcctggctgaggccatgagccaggtgaccaactccgctaccatcatgatgcagcgcggcaactttcggaaccaacgcaagatcgtcaagtgcttcaactgtggcaaagaagggcacacagcccgcaactgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcagctcaaggaggctctcctggacaccggagcagacgacaccgtgctggaggagatgtcgttgccaggccgctggaagccgaagatgatcgggggaatcggcggtttcatcaaggtgcgccagtatgaccagatcctcatcgaaatctgcggccacaaggctatcggtaccgtgctggtgggccccacacccgtcaacatcatcggacgcaacctgttgacgcagatcggttgcacgctgaacttccccattagccctatcgagacggtaccggtgaagctgaagcccgggatggacggcccgaaggtcaagcaatggccattgacagaggagaagatcaaggcactggtggagatttgcacagagatggaaaaggaagggaaaatctccaagattgggcctgagaacccgtacaacacgccggtgttcgcaatcaagaagaaggactcgacgaaatggcgcaagctggtggacttccgcgagctgaacaagcgcacgcaagacttctgggaggttcagctgggcatcccgcaccccgcagggctgaagaagaagaaatccgtgaccgtactggatgtgggtgatgcctacttctccgttcccctggacgaagacttcaggaagtacactgccttcacaatcccttcgatcaacaacgagacaccggggattcgatatcagtacaacgtgctgccccagggctggaaaggctctcccgcaatcttccagagtagcatgaccaaaatcctggagcctttccgcaaacagaaccccgacatcgtcatctatcagtacatggatgacttgtacgtgggctctgatctagagatagggcagcaccgcaccaagatcgaggagctgcgccagcacctgttgaggtggggactgaccacacccgacaagaagcaccagaaggagcctcccttcctctggatgggttacgagctgcaccctgacaaatggaccgtgcagcctatcgtgctgccagagaaagacagctggactgtcaacgacatacagaagctggtggggaagttgaactgggccagtcagatttacccagggattaaggtgaggcagctgtgcaaactcctccgcggaaccaaggcactcacagaggtgatccccctaaccgaggaggccgagctcgaactggcagaaaaccgagagatcctaaaggagcccgtgcacggcgtgtactatgacccctccaaggacctgatcgccgagatccagaagcaggggcaaggccagtggacctatcagatttaccaggagcccttcaagaacctgaagaccggcaagtacgcccggatgaggggtgcccacactaacgacgtcaagcagctgaccgaggccgtgcagaagatcaccaccgaaagcatcgtgatctggggaaagactcctaagttcaagctgcccatccagaaggaaacctgggaaacctggtggacagagtattggcaggccacctggattcctgagtgggagttcgtcaacacccctcccctggtgaagctgtggtaccagctggagaaggagcccatagtgggcgccgaaaccttctacgtggatggggccgctaacagggagactaagctgggcaaagccggatacgtcactaaccggggcagacagaaggttgtcaccctcactgacaccaccaaccagaagactgagctgcaggccatttacctcgctttgcaggactcgggcctggaggtgaacatcgtgacagactctcagtatgccctgggcatcattcaagcccagccagaccagagtgagtccgagctggtcaatcagatcatcgagcagctgatcaagaaggaaaaggtctatctggcctgggtacccgcccacaaaggcattggcggcaatgagcaggtcgacaagctggtctcggctggcatcaggaaggtgctattcctggatggcatcgacaaggcccaggacgagcacgagaaataccacagcaactggcgggccatggctagcgacttcaacctgccccctgtggtggccaaagagatcgtggccagctgtgacaagtgtcagctcaagggcgaagccatgcatggccaggtggactgtagccccggcatctggcaactcgattgcacccatctggagggcaaggttatcctggtagccgtccatgtggccagtggctacatcgaggccgaggtcattcccgccgaaacagggcaggagacagcctacttcctcctgaagctggcaggccggtggccagtgaagaccatccatactgacaatggcagcaatttcaccagtgctacggttaaggccgcctgctggtgggcgggaatcaagcaggagttcgggatcccctacaatccccagagtcagggcgtcgtcgagtctatgaataaggagttaaagaagattatcggccaggtcagagatcaggctgagcatctcaagaccgcggtccaaatggcggtattcatccacaatttcaagcggaagggggggattggggggtacagtgcgggggagcggatcgtggacatcatcgcgaccgacatccagactaaggagctgcaaaagcagattaccaagattcagaatttccgggtctactacagggacagcagaaatcccctctggaaaggcccagcgaagctcctctggaagggtgagggggcagtagtgatccaggataatagcgacatcaaggtggtgcccagaagaaaggcgaagatcattagggattatggcaaacagatggcgggtgatgattgcgtggcgagcagacaggatgaggattag

SEQ ID NO: 16 - DNA, 4658 bp, Artificial Sequence
  Description of Artificial Sequence Codon optimised EIAV gag-pol
atgggcgatcccctcacctggtccaaagccctgaagaaactggaaaaagtcaccgttcagggtagccaaaagcttaccacaggcaattgcaactgggcattgtccctggtggatcttttccacgacactaatttcgttaaggagaaagattggcaactcagagacgtgatccccctcttggaggacgtgacccaaacattgtctgggcaggagcgcgaagctttcgagcgcacctggtgggccatcagcgcagtcaaaatggggctgcaaatcaacaacgtggttgacggtaaagctagctttcaactgctccgcgctaagtacgagaagaaaaccgccaacaagaaacaatccgaacctagcgaggagtacccaattatgatcgacggcgccggcaataggaacttccgcccactgactcccaggggctataccacctgggtcaacaccatccagacaaacggacttttgaacgaagcctcccagaacctgttcggcatcctgtctgtggactgcacctccgaagaaatgaatgcttttctcgacgtggtgccaggacaggctggacagaaacagatcctgctcgatgccattgacaagatcgccgacgactgggataatcgccaccccctgccaaacgcccctctggtggctcccccacaggggcctatccctatgaccgctaggttcattaggggactgggggtgccccgcgaacgccagatggagccagcatttgaccaatttaggcagacctacagacagtggatcatcgaagccatgagcgaggggattaaagtcatgatcggaaagcccaaggcacagaacatcaggcagggggccaaggaaccataccctgagtttgtcgacaggcttctgtcccagattaaatccgaaggccaccctcaggagatctccaagttcttgacagacacactgactatccaaaatgcaaatgaagagtgcagaaacgccatgaggcacctcagacctgaagataccctggaggagaaaatgtacgcatgtcgcgacattggcactaccaagcaaaagatgatgctgctcgccaaggctctgcaaaccggcctggctggtccattcaaaggaggagcactgaagggaggtccattgaaagctgcacaaacatgttataattgtgggaagccaggacatttatctagtcaatgtagagcacctaaagtctgttttaaatgtaaacagcctggacatttctcaaagcaatgcagaagtgttccaaaaaacgggaagcaaggggctcaagggaggccccagaaacaaactttcccgatacaacagaagagtcagcacaacaaatctgttgtacaagagactcctcagactcaaaatctgtacccagatctgagcgaaataaaaaaggaatacaatgtcaaggagaaggatcaagtagaggatctcaacctggacagtttgtgggagtaacatacaatctcgagaagaggcccactaccatcgtcctgatcaatgacacccctcttaatgtgctgctggacaccggagccgacaccagcgttctcactactgctcactataacagactgaaatacagaggaaggaaataccagggcacaggcatcatcggcgttggaggcaacgtcgaaaccttttccactcctgtcaccatcaaaaagaaggggagacacattaaaaccagaatgctggtcgccgacatccccgtcaccatccttggcagagacattctccaggacctgggcgctaaactcgtgctggcacaactgtctaaggaaatcaagttccgcaagatcgagctgaaagagggcacaatgggtccaaaaatcccccagtggcccctgaccaaagagaagcttgagggcgctaaggaaatcgtgcagcgcctgctttctgagggcaagattagcgaggccagcgacaataacccttacaacagccccatctttgtgattaagaaaaggagcggcaaatggagactcctgcaggacctgagggaactcaacaagaccgtccaggtcggaactgagatctctcgcggactgcctcaccccggcggcctgattaaatgcaagcacatgacagtccttgacattggagacgcttattttaccatccccctcgatcctgaatttcgcccctatactgcttttaccatccccagcatcaatcaccaggagcccgataaacgctatgtgtggaagtgcctcccccagggatttgtgcttagcccctacatttaccagaagacacttcaagagatcctccaacctttccgcgaaagatacccagaggttcaactctaccaatatatggacgacctgttcatggggtccaacgggtctaagaagcagcacaaggaactcatcatcgaactgagggcaatcctcctggagaaaggcttcgagacacccgacgacaagctgcaagaagttcctccatatagctggctgggctaccagctttgccctgaaaactggaaagtccagaagatgcagttggatatggtcaagaacccaacactgaacgacgtccagaagctcatgggcaatattacctggatgagctccggaatccctgggcttaccgttaagcacattgccgcaactacaaaaggatgcctggagttgaaccagaaggtcatttggacagaggaagctcagaaggaactggaggagaataatgaaaagattaagaatgctcaagggctccaatactacaatcccgaagaagaaatgttgtgcgaggtcgaaatcactaagaactacgaagccacctatgtcatcaaacagtcccaaggcatcttgtgggccggaaagaaaatcatgaaggccaacaaaggctggtccaccgttaaaaatctgatgctcctgctccagcacgtcgccaccgagtctatcacccgcgtcggcaagtgccccaccttcaaagttcccttcactaaggagcaggtgatgtgggagatgcaaaaaggctggtactactcttggcttcccgagatcgtctacacccaccaagtggtgcacgacgactggagaatgaagcttgtcgaggagcccactagcggaattacaatctataccgacggcggaaagcaaaacggagagggaatcgctgcatacgtcacatctaacggccgcaccaagcaaaagaggctcggccctgtcactcaccaggtggctgagaggatggctatccagatggcccttgaggacactagagacaagcaggtgaacattgtgactgacagctactactgctggaaaaacatcacagagggccttggcctggagggaccccagtctccctggtggcctatcatccagaatatccgcgaaaaggaaattgtctatttcgcctgggtgcctggacacaaaggaatttacggcaaccaactcgccgatgaagccgccaaaattaaagaggaaatcatgcttgcctaccagggcacacagattaaggagaagagagacgaggacgctggctttgacctgtgtgtgccatacgacatcatgattcccgttagcgacacaaagatcattccaaccgatgtcaagatccaggtgccacccaattcatttggttgggtgaccggaaagtccagcatggctaagcagggtcttctgattaacgggggaatcattgatgaaggatacaccggcgaaatccaggtgatctgcacaaatatcggcaaaagcaatattaagcttatcgaagggcagaagttcgctcaactcatcatcctccagcaccacagcaattcaagacaaccttgggacgaaaacaagattagccagagaggtgacaagggcttcggcagcacaggtgtgttctgggtggagaacatccaggaagcacaggacgagcacgagaattggcacacctcccctaagattttggcccgcaattacaagatcccactgactgtggctaagcagatcacacaggaatgcccccactgcaccaaacaaggttctggccccgccggctgcgtgatgaggtcccccaatcactggcaggcagattgcacccacctcgacaacaaaattatcctgaccttcgtggagagcaattccggctacatccacgcaacactcctctccaaggaaaatgcattgtgcacctccctcgcaattctggaatgggccaggctgttctctccaaaatccctgcacaccgacaacggcaccaactttgtggctgaacctgtggtgaatctgctgaagttcctgaaaatcgcccacaccactggcattccctatcaccctgaaagccagggcattgtcgagagggccaacagaactctgaaagaaaagatccaatctcacagagacaatacacagacattggaggccgcacttcagctcgcccttatcacctgcaacaaaggaagagaaagcatgggcggccagaccccctgggaggtcttcatcactaaccaggcccaggtcatccatgaaaagctgctcttgcagcaggcccagtcctccaaaaagttctgcttttataagatccccggtgagcacgactggaaaggtcctacaagagttttgtggaaaggagacggcgcagttgtggtgaacgatgagggcaaggggatcatcgctgtgcccctgacacgcaccaagcttctcatcaagccaaactga

SEQ ID NO: 17 - DNA, 10392 bp, Artificial Sequence
  Description of Artificial Sequence pIRES1hygESYNGP
aattcgccaccatgggcgatcccctcacctggtccaaagccctgaagaaactggaaaaagtcaccgttcagggtagccaaaagcttaccacaggcaattgcaactgggcattgtccctggtggatcttttccacgacactaatttcgttaaggagaaagattggcaactcagagacgtgatccccctcttggaggacgtgacccaaacattgtctgggcaggagcgcgaagctttcgagcgcacctggtgggccatcagcgcagtcaaaatggggctgcaaatcaacaacgtggttgacggtaaagctagctttcaactgctccgcgctaagtacgagaagaaaaccgccaacaagaaacaatccgaacctagcgaggagtacccaattatgatcgacggcgccggcaataggaacttccgcccactgactcccaggggctataccacctgggtcaacaccatccagacaaacggacttttgaacgaagcctcccagaacctgttcggcatcctgtctgtggactgcacctccgaagaaatgaatgcttttctcgacgtggtgccaggacaggctggacagaaacagatcctgctcgatgccattgacaagatcgccgacgactgggataatcgccaccccctgccaaacgcccctctggtggctcccccacaggggcctatccctatgaccgctaggttcattaggggactgggggtgccccgcgaacgccagatggagccagcatttgaccaatttaggcagacctacagacagtggatcatcgaagccatgagcgaggggattaaagtcatgatcggaaagcccaaggcacagaacatcaggcagggggccaaggaaccataccctgagtttgtcgacaggcttctgtcccagattaaatccgaaggccaccctcaggagatctccaagttcttgacagacacactgactatccaaaatgcaaatgaagagtgcagaaacgccatgaggcacctcagacctgaagataccctggaggagaaaatgtacgcatgtcgcgacattggcactaccaagcaaaagatgatgctgctcgccaaggctctgcaaaccggcctggctggtccattcaaaggaggagcactgaagggaggtccattgaaagctgcacaaacatgttataattgtgggaagccaggacatttatctagtcaatgtagagcacctaaagtctgttttaaatgtaaacagcctggacatttctcaaagcaatgcagaagtgttccaaaaaacgggaagcaaggggctcaagggaggccccagaaacaaactttcccgatacaacagaagagtcagcacaacaaatctgttgtacaagagactcctcagactcaaaatctgtacccagatctgagcgaaataaaaaaggaatacaatgtcaaggagaaggatcaagtagaggatctcaacctggacagtttgtgggagtaacatacaatctcgagaagaggcccactaccatcgtcctgatcaatgacacccctcttaatgtgctgctggacaccggagccgacaccagcgttctcactactgctcactataacagactgaaatacagaggaaggaaataccagggcacaggcatcatcggcgttggaggcaacgtcgaaaccttttccactcctgtcaccatcaaaaagaaggggagacacattaaaaccagaatgctggtcgccgacatccccgtcaccatccttggcagagacattctccaggacctgggcgctaaactcgtgctggcacaactgtctaaggaaatcaagttccgcaagatcgagctgaaagagggcacaatgggtccaaaaatcccccagtggcccctgaccaaagagaagcttgagggcgctaaggaaatcgtgcagcgcctgctttctgagggcaagattagcgaggccagcgacaataacccttacaacagccccatctttgtgattaagaaaaggagcggcaaatggagactcctgcaggacctgagggaactcaacaagaccgtccaggtcggaactgagatctctcgcggactgcctcaccccggcggcctgattaaatgcaagcacatgacagtccttgacattggagacgcttattttaccatccccctcgatcctgaatttcgcccctatactgcttttaccatccccagcatcaatcaccaggagcccgataaacgctatgtgtggaagtgcctcccccagggatttgtgcttagcccctacatttaccagaagacacttcaagagatcctccaacctttccgcgaaagatacccagaggttcaactctaccaatatatggacgacctgttcatggggtccaacgggtctaagaagcagcacaaggaactcatcatcgaactgagggcaatcctcctggagaaaggcttcgagacacccgacgacaagctgcaagaagttcctccatatagctggctgggctaccagctttgccctgaaaactggaaagtccagaagatgcagttggatatggtcaagaacccaacactgaacgacgtccagaagctcatgggcaatattacctggatgagctccggaatccctgggcttaccgttaagcacattgccgcaactacaaaaggatgcctggagttgaaccagaaggtcatttggacagaggaagctcagaaggaactggaggagaataatgaaaagattaagaatgctcaagggctccaatactacaatcccgaagaagaaatgttgtgcgaggtcgaaatcactaagaactacgaagccacctatgtcatcaaacagtcccaaggcatcttgtgggccggaaagaaaatcatgaaggccaacaaaggctggtccaccgttaaaaatctgatgctcctgctccagcacgtcgccaccgagtctatcacccgcgtcggcaagtgccccaccttcaaagttcccttcactaaggagcaggtgatgtgggagatgcaaaaaggctggtactactcttggcttcccgagatcgtctacacccaccaagtggtgcacgacgactggagaatgaagcttgtcgaggagcccactagcggaattacaatctataccgacggcggaaagcaaaacggagagggaatcgctgcatacgtcacatctaacggccgcaccaagcaaaagaggctcggccctgtcactcaccaggtggctgagaggatggctatccagatggcccttgaggacactagagacaagcaggtgaacattgtgactgacagctactactgctggaaaaacatcacagagggccttggcctggagggaccccagtctccctggtggcctatcatccagaatatccgcgaaaaggaaattgtctatttcgcctgggtgcctggacacaaaggaatttacggcaaccaactcgccgatgaagccgccaaaattaaagaggaaatcatgcttgcctaccagggcacacagattaaggagaagagagacgaggacgctggctttgacctgtgtgtgccatacgacatcatgattcccgttagcgacacaaagatcattccaaccgatgtcaagatccaggtgccacccaattcatttggttgggtgaccggaaagtccagcatggctaagcagggtcttctgattaacgggggaatcattgatgaaggatacaccggcgaaatccaggtgatctgcacaaatatcggcaaaagcaatattaagcttatcgaagggcagaagttcgctcaactcatcatcctccagcaccacagcaattcaagacaaccttgggacgaaaacaagattagccagagaggtgacaagggcttcggcagcacaggtgtgttctgggtggagaacatccaggaagcacaggacgagcacgagaattggcacacctcccctaagattttggcccgcaattacaagatcccactgactgtggctaagcagatcacacaggaatgcccccactgcaccaaacaaggttctggccccgccggctgcgtgatgaggtcccccaatcactggcaggcagattgcacccacctcgacaacaaaattatcctgaccttcgtggagagcaattccggctacatccacgcaacactcctctccaaggaaaatgcattgtgcacctccctcgcaattctggaatgggccaggctgttctctccaaaatccctgcacaccgacaacggcaccaactttgtggctgaacctgtggtgaatctgctgaagttcctgaaaatcgcccacaccactggcattccctatcaccctgaaagccagggcattgtcgagagggccaacagaactctgaaagaaaagatccaatctcacagagacaatacacagacattggaggccgcacttcagctcgcccttatcacctgcaacaaaggaagagaaagcatgggcggccagaccccctgggaggtcttcatcactaaccaggcccaggtcatccatgaaaagctgctcttgcagcaggcccagtcctccaaaaagttctgcttttataagatccccggtgagcacgactggaaaggtcctacaagagttttgtggaaaggagacggcgcagttgtggtgaacgatgagggcaaggggatcatcgctgtgcccctgacacgcaccaagcttctcatcaagccaaactgaacccggggcggccgcactagaggaattcgcccctctccctcccccccccctaacgttactggccgaagccgcttggaataaggccggtgtgtgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtagtcaacaaggggctgaaggatgcccagaaggtaccccattgtatgggaatctgatctggggcctcggtgcacatgctttacatgtgtttagtcgaggttaaaaaagctctaggccccccgaaccacggggacgtggttttcctttgaaaaacacgatgataagcttgccacaaccccgtaccaaagatggatagatccggaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgaacaagagctgatttcgagaacgcctcagccagcaactcgcgcgagcctagcaaggcaaatgcgagagaacggccttacgcttggtggcacagttctcgtccacagttcgctaagctcgctcggctgggtcgcgggagggccggtcgcagtgattcaggcccttctggattgtgttggtccccagggcacgattgtcatgcccacgcactcgggtgatctgactgatcccgcagattggagatcgccgcccgtgcctgccgattgggtgcagatctagagctcgctgatcagcctcgactgtgcctctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagtgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccactagtaacggccgccagtgtgctggaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgatcgagca

SEQ ID NO: 18 - DNA, 10114 bp, Artificial Sequence
  Description of Artificial Sequence pESDSYNGP
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagagaattccaggtaagatgggcgatcccctcacctggtccaaagccctgaagaaactggaaaaagtcaccgttcagggtagccaaaagcttaccacaggcaattgcaactgggcattgtccctggtggatcttttccacgacactaatttcgttaaggagaaagattggcaactcagagacgtgatccccctcttggaggacgtgacccaaacattgtctgggcaggagcgcgaagctttcgagcgcacctggtgggccatcagcgcagtcaaaatggggctgcaaatcaacaacgtggttgacggtaaagctagctttcaactgctccgcgctaagtacgagaagaaaaccgccaacaagaaacaatccgaacctagcgaggagtacccaattatgatcgacggcgccggcaataggaacttccgcccactgactcccaggggctataccacctgggtcaacaccatccagacaaacggacttttgaacgaagcctcccagaacctgttcggcatcctgtctgtggactgcacctccgaagaaatgaatgcttttctcgacgtggtgccaggacaggctggacagaaacagatcctgctcgatgccattgacaagatcgccgacgactgggataatcgccaccccctgccaaacgcccctctggtggctcccccacaggggcctatccctatgaccgctaggttcattaggggactgggggtgccccgcgaacgccagatggagccagcatttgaccaatttaggcagacctacagacagtggatcatcgaagccatgagcgaggggattaaagtcatgatcggaaagcccaaggcacagaacatcaggcagggggccaaggaaccataccctgagtttgtcgacaggcttctgtcccagattaaatccgaaggccaccctcaggagatctccaagttcttgacagacacactgactatccaaaatgcaaatgaagagtgcagaaacgccatgaggcacctcagacctgaagataccctggaggagaaaatgtacgcatgtcgcgacattggcactaccaagcaaaagatgatgctgctcgccaaggctctgcaaaccggcctggctggtccattcaaaggaggagcactgaagggaggtccattgaaagctgcacaaacatgttataattgtgggaagccaggacatttatctagtcaatgtagagcacctaaagtctgttttaaatgtaaacagcctggacatttctcaaagcaatgcagaagtgttccaaaaaacgggaagcaaggggctcaagggaggccccagaaacaaactttcccgatacaacagaagagtcagcacaacaaatctgttgtacaagagactcctcagactcaaaatctgtacccagatctgagcgaaataaaaaaggaatacaatgtcaaggagaaggatcaagtagaggatctcaacctggacagtttgtgggagtaacatacaatctcgagaagaggcccactaccatcgtcctgatcaatgacacccctcttaatgtgctgctggacaccggagccgacaccagcgttctcactactgctcactataacagactgaaatacagaggaaggaaataccagggcacaggcatcatcggcgttggaggcaacgtcgaaaccttttccactcctgtcaccatcaaaaagaaggggagacacattaaaaccagaatgctggtcgccgacatccccgtcaccatccttggcagagacattctccaggacctgggcgctaaactcgtgctggcacaactgtctaaggaaatcaagttccgcaagatcgagctgaaagagggcacaatgggtccaaaaatcccccagtggcccctgaccaaagagaagcttgagggcgctaaggaaatcgtgcagcgcctgctttctgagggcaagattagcgaggccagcgacaataacccttacaacagccccatctttgtgattaagaaaaggagcggcaaatggagactcctgcaggacctgagggaactcaacaagaccgtccaggtcggaactgagatctctcgcggactgcctcaccccggcggcctgattaaatgcaagcacatgacagtccttgacattggagacgcttattttaccatccccctcgatcctgaatttcgcccctatactgcttttaccatccccagcatcaatcaccaggagcccgataaacgctatgtgtggaagtgcctcccccagggatttgtgcttagcccctacatttaccagaagacacttcaagagatcctccaacctttccgcgaaagatacccagaggttcaactctaccaatatatggacgacctgttcatggggtccaacgggtctaagaagcagcacaaggaactcatcatcgaactgagggcaatcctcctggagaaaggcttcgagacacccgacgacaagctgcaagaagttcctccatatagctggctgggctaccagctttgccctgaaaactggaaagtccagaagatgcagttggatatggtcaagaacccaacactgaacgacgtccagaagctcatgggcaatattacctggatgagctccggaatccctgggcttaccgttaagcacattgccgcaactacaaaaggatgcctggagttgaaccagaaggtcatttggacagaggaagctcagaaggaactggaggagaataatgaaaagattaagaatgctcaagggctccaatactacaatcccgaagaagaaatgttgtgcgaggtcgaaatcactaagaactacgaagccacctatgtcatcaaacagtcccaaggcatcttgtgggccggaaagaaaatcatgaaggccaacaaaggctggtccaccgttaaaaatctgatgctcctgctccagcacgtcgccaccgagtctatcacccgcgtcggcaagtgccccaccttcaaagttcccttcactaaggagcaggtgatgtgggagatgcaaaaaggctggtactactcttggcttcccgagatcgtctacacccaccaagtggtgcacgacgactggagaatgaagcttgtcgaggagcccactagcggaattacaatctataccgacggcggaaagcaaaacggagagggaatcgctgcatacgtcacatctaacggccgcaccaagcaaaagaggctcggccctgtcactcaccaggtggctgagaggatggctatccagatggcccttgaggacactagagacaagcaggtgaacattgtgactgacagctactactgctggaaaaacatcacagagggccttggcctggagggaccccagtctccctggtggcctatcatccagaatatccgcgaaaaggaaattgtctatttcgcctgggtgcctggacacaaaggaatttacggcaaccaactcgccgatgaagccgccaaaattaaagaggaaatcatgcttgcctaccagggcacacagattaaggagaagagagacgaggacgctggctttgacctgtgtgtgccatacgacatcatgattcccgttagcgacacaaagatcattccaaccgatgtcaagatccaggtgccacccaattcatttggttgggtgaccggaaagtccagcatggctaagcagggtcttctgattaacgggggaatcattgatgaaggatacaccggcgaaatccaggtgatctgcacaaatatcggcaaaagcaatattaagcttatcgaagggcagaagttcgctcaactcatcatcctccagcaccacagcaattcaagacaaccttgggacgaaaacaagattagccagagaggtgacaagggcttcggcagcacaggtgtgttctgggtggagaacatccaggaagcacaggacgagcacgagaattggcacacctcccctaagattttggcccgcaattacaagatcccactgactgtggctaagcagatcacacaggaatgcccccactgcaccaaacaaggttctggccccgccggctgcgtgatgaggtcccccaatcactggcaggcagattgcacccacctcgacaacaaaattatcctgaccttcgtggagagcaattccggctacatccacgcaacactcctctccaaggaaaatgcattgtgcacctccctcgcaattctggaatgggccaggctgttctctccaaaatccctgcacaccgacaacggcaccaactttgtggctgaacctgtggtgaatctgctgaagttcctgaaaatcgcccacaccactggcattccctatcaccctgaaagccagggcattgtcgagagggccaacagaactctgaaagaaaagatccaatctcacagagacaatacacagacattggaggccgcacttcagctcgcccttatcacctgcaacaaaggaagagaaagcatgggcggccagaccccctgggaggtcttcatcactaaccaggcccaggtcatccatgaaaagctgctcttgcagcaggcccagtcctccaaaaagttctgcttttataagatccccggtgagcacgactggaaaggtcctacaagagttttgtggaaaggagacggcgcagttgtggtgaacgatgagggcaaggggatcatcgctgtgcccctgacacgcaccaagcttctcatcaagccaaactgaacccggggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 19 - DNA, 10384 bp, Artificial Sequence
  Description of Artificial Sequence pONY8.3G FB29
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatctctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcggccccaagcttgttgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcgacctgcaggcatgcaagcttcagctgctcgagggggggcccggtacccagcttttgttccctttagtgagggttaattgcgcgggaagtatttatcactaatcaagcacaagtaatacatgagaaacttttactacagcaagcacaatcctccaaaaaattttgtttttacaaaatccctggtgaacatgattggaagggacctactagggtgctgtggaagggtgatggtgcagtagtagttaatgatgaaggaaagggaataattgctgtaccattaaccaggactaagttactaataaaaccaaattgagtattgttgcaggaagcaagacccaactaccattgtcagctgtgtttcctgacctcaatatttgttataaggtttgatatgaatcccagggggaatctcaacccctattacccaacagtcagaaaaatctaagtgtgaggagaacacaatgtttcaaccttattgttataataatgacagtaagaacagcatggcagaatcgaaggaagcaagagaccaagaatgaacctgaaagaagaatctaaagaagaaaaaagaagaaatgactggtggaaaataggtatgtttctgttatgcttagcaggaactactggaggaatactttggtggtatgaaggactcccacagcaacattatatagggttggtggcgatagggggaagattaaacggatctggccaatcaaatgctatagaatgctggggttccttcccggggtgtagaccatttcaaaattacttcagttatgagaccaatagaagcatgcatatggataataatactgctacattattagaagctttaaccaatataactgctctataaataacaaaacagaattagaaacatggaagttagtaaagacttctggcataactcctttacctatttcttctgaagctaacactggactaattagacataagagagattttggtataagtgcaatagtggcagctattgtagccgctactgctattgctgctagcgctactatgtcttatgttgctctaactgaggttaacaaaataatggaagtacaaaatcatacttttgaggtagaaaatagtactctaaatggtatggatttaatagaacgacaaataaagatattatatgctatgattcttcaaacacatgcagatgttcaactgttaaaggaaagacaacaggtagaggagacatttaatttaattggatgtatagaaagaacacatgtattttgtcatactggtcatccctggaatatgtcatggggacatttaaatgagtcaacacaatgggatgactgggtaagcaaaatggaagatttaaatcaagagatactaactacacttcatggagccaggaacaatttggcacaatccatgataacattcaatacaccagatagtatagctcaatttggaaaagacctttggagtcatattggaaattggattcctggattgggagcttccattataaaatatatagtgatgtttttgcttatttatttgttactaacctcttcgcctaagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgactgttgcattaaagcccaagaaggaactctcgctatcccttgctgtggatttcccttatggctattttggggactagtaattatagtaggacgcatagcaggctatggattacgtggactcgctgttataataaggatttgtattagaggcttaaatttgatatttgaaataatcagaaaaatgcttgattatattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgttaacttcctgtttttacagtatataagtgcttgtattctgacaattgggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagtgatgcccgggcggccgaggcggcctacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagcccggatcgatccttatcggattttaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgaagcattaaccctcactaaagggaagcggccgcccgggtcgacttcacaggtgtttgcggcgtcttttggagtctccgggcctcaagacgcgggggctgctctgctcgccccacagcctttcttgtgccctctggtagcctccccatgcggagaaatcgcccctctggtcctcgcggaagtagagctccctccagatgccgcgattcacctctcccagctctttagcggcttgttgcacgcccctaattctccattccagcctttcttggaggacctcggcttgcaaaatctggcccctaatccacctatcccttctggagggtgtgtgctgggtgggaccggggccgaggtgtcttctggcgatgcaggtctggctaggaatcttctcctcgggcagggactgtctcagcacgcggcaccactggtccccctccagggggccttgtgggtcgatcttccaccagtcgttgcggcgcttctcctctttgctctcttccttgaggttcatctcttgatccctggcctccttgctctcagccatggtggcgaattctcgaggctagcctcccggtggtgggtcggtggtccctgggcaggggtctccagatcccggacgagcccccaaatgaaagacccccgagacgggtagtcaatcactctgaggagaccctcccaaggaacagcgagaccacgagtcggatgcaacagcaagaggatttattggatacacgggtacccgggcgactcagtctatcggaggactggcgcgccgagtgaggggttgtgagctcttttatagagctcgggaagcagaagcgcgcgaacagaagcgagaagcaggctgattggttaattcaaataaggcacagggtcatttcaggtccttgggggagcctggaaacatctgatgggtcttaagaaactgctgagggttgggccatatctggggaccatctgttcttggccccgggccggggccgaaccgcggtgaccatctgttcttggccccgggccggggccgaaactgctcaccgcagatatcctgtttggcccaacgttagctgttttcgtgtacccgcccttgatctgaacttctctattcttggtttggtatttttccatgccttgcaaaatggcgttactgcggctatcaggctaagcaatttgagatctggccgaggcggcctactctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtataacttcgtataatgtatgctatacgaagttatacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgataacttcgtataatgtatgctatacgaagttatcactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

SEQ ID NO: 20 - DNA, 21 bp, Artificial Sequence
  Description of Artificial Sequence Sequence flanking the codon-optimised EIAV gag/pol ORF
tctagagaattcgccaccatg

SEQ ID NO: 21 - DNA, 18 bp, Artificial Sequence
  Description of Artificial Sequence Sequence flanking the codon-optimised EIAV gag/pol ORF
tgaacccggggcggccgc

SEQ ID NO: 22 - DNA, 11 bp, Artificial Sequence
  Description of Artificial Sequence pEV53B
caggtaagatg

SEQ ID NO: 23 - DNA, 42 bp, Artificial Sequence
  Description of Artificial Sequence Primer
ggctagagaattccaggtaagatgggcgatcccctcacctgg

SEQ ID NO: 24 - DNA, 22 bp, Artificial Sequence
  Description of Artificial Sequence Primer
ttgggtactcctcgctaggttc

SEQ ID NO: 25 - DNA, 4307 bp, Artificial Sequence; identical to SEQ ID NO: 15
  Description of Artificial Sequence Codon optimised gag-pol sequence (pSYNGP)

SEQ ID NO: 26 - DNA, 4307 bp, Artificial Sequence
  Description of Artificial Sequence pGP-RRE3
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaagaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcagatcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaaatatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcctttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatacagacaatggcagcaatttcaccagtgctacggttaaggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattag

SEQ ID NO: 27 - DNA, 4658 bp, Equine infectious anemia virus
atgggagaccctttgacatggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatccaatcatgatagatggggctggaaacagaaattttagacctctaacacctagaggatatactacttgggtgaataccatacagacaaatggtctattaaatgaagctagtcaaaacttatttgggatattatcagtagactgtacttctgaagaaatgaatgcatttttggatgtggtacctggccaggcaggacaaaagcagatattacttgatgcaattgataagatagcagatgattgggataatagacatccattaccgaatgctccactggtggcaccaccacaagggcctattcccatgacagcaaggtttattagaggtttaggagtacctagagaaagacagatggagcctgcttttgatcagtttaggcagacatatagacaatggataatagaagccatgtcagaaggcatcaaagtgatgattggaaaacctaaagctcaaaatattaggcaaggagctaaggaaccttacccagaatttgtagacagactattatcccaaataaaaagtgagggacatccacaagagatttcaaaattcttgactgatacactgactattcagaacgcaaatgaggaatgtagaaatgctatgagacatttaagaccagaggatacattagaagagaaaatgtatgcttgcagagacattggaactacaaaacaaaagatgatgttattggcaaaagcacttcagactggtcttgcgggcccatttaaaggtggagccttgaaaggagggccactaaaggcagcacaaacatgttataactgtgggaagccaggacatttatctagtcaatgtagagcacctaaagtctgttttaaatgtaaacagcctggacatttctcaaagcaatgcagaagtgttccaaaaaacgggaagcaaggggctcaagggaggccccagaaacaaactttcccgatacaacagaagagtcagcacaacaaatctgttgtacaagagactcctcagactcaaaatctgtacccagatctgagcgaaataaaaaaggaatacaatgtcaaggagaaggatcaagtagaggatctcaacctggacagtttgtgggagtaacatataatctagagaaaaggcctactacaatagtattaattaatgatactcccttaaatgtactgttagacacaggagcagatacttcagtgttgactactgcacattataataggttaaaatatagagggagaaaatatcaagggacgggaataataggagtgggaggaaatgtggaaacattttctacgcctgtgactataaagaaaaagggtagacacattaagacaagaatgctagtggcagatattccagtgactattttgggacgagatattcttcaggacttaggtgcaaaattggttttggcacagctctccaaggaaataaaatttagaaaaatagagttaaaagagggcacaatggggccaaaaattcctcaatggccactcactaaggagaaactagaaggggccaaagagatagtccaaagactattgtcagagggaaaaatatcagaagctagtgacaataatccttataattcacccatatttgtaataaaaaagaggtctggcaaatggaggttattacaagatctgagagaattaaacaaaacagtacaagtaggaacggaaatatccagaggattgcctcacccgggaggattaattaaatgtaaacacatgactgtattagatattggagatgcatatttcactatacccttagatccagagtttagaccatatacagctttcactattccctccattaatcatcaagaaccagataaaagatatgtgtggaaatgtttaccacaaggattcgtgttgagcccatatatatatcagaaaacattacaggaaattttacaaccttttagggaaagatatcctgaagtacaattgtatcaatatatggatgatttgttcatgggaagtaatggttctaaaaaacaacacaaagagttaatcatagaattaagggcgatcttactggaaaagggttttgagacaccagatgataaattacaagaagtgccaccttatagctggctaggttatcaactttgtcctgaaaattggaaagtacaaaaaatgcaattagacatggtaaagaatccaacccttaatgatgtgcaaaaattaatggggaatataacatggatgagctcagggatcccagggttgacagtaaaacacattgcagctactactaagggatgtttagagttgaatcaaaaagtaatttggacggaagaggcacaaaaagagttagaagaaaataatgagaagattaaaaatgctcaagggttacaatattataatccagaagaagaaatgttatgtgaggttgaaattacaaaaaattatgaggcaacttatgttataaaacaatcacaaggaatcctatgggcaggtaaaaagattatgaaggctaataagggatggtcaacagtaaaaaatttaatgttattgttgcaacatgtggcaacagaaagtattactagagtaggaaaatgtccaacgtttaaggtaccatttaccaaagagcaagtaatgtgggaaatgcaaaaaggatggtattattcttggctcccagaaatagtatatacacatcaagtagttcatgatgattggagaatgaaattggtagaagaacctacatcaggaataacaatatacactgatgggggaaaacaaaatggagaaggaatagcagcttatgtgaccagtaatgggagaactaaacagaaaaggttaggacctgtcactcatcaagttgctgaaagaatggcaatacaaatggcattagaggataccagagataaacaagtaaatatagtaactgatagttattattgttggaaaaatattacagaaggattaggtttagaaggaccacaaagtccttggtggcctataatacaaaatatacgagaaaaagagatagtttattttgcttgggtacctggtcacaaagggatatatggtaatcaattggcagatgaagccgcaaaaataaaagaagaaatcatgctagcataccaaggcacacaaattaaagagaaaagagatgaagatgcagggtttgacttatgtgttccttatgacatcatgatacctgtatctgacacaaaaatcatacccacagatgtaaaaattcaagttcctcctaatagctttggatgggtcactgggaaatcatcaatggcaaaacaggggttattaattaatggaggaataattgatgaaggatatacaggagaaatacaagtgatatgtactaatattggaaaaagtaatattaaattaatagagggacaaaaatttgcacaattaattatactacagcatcactcaaattccagacagccttgggatgaaaataaaatatctcagagaggggataaaggatttggaagtacaggagtattctgggtagaaaatattcaggaagcacaagatgaacatgagaattggcatacatcaccaaagatattggcaagaaattataagataccattgactgtagcaaaacagataactcaagaatgtcctcattgcactaagcaaggatcaggacctgcaggttgtgtcatgagatctcctaatcattggcaggcagattgcacacatttggacaataagataatattgacttttgtagagtcaaattcaggatacatacatgctacattattgtcaaaagaaaatgcattatgtacttcattggctattttagaatgggcaagattgttttcaccaaagtccttacacacagataacggcactaattttgtggcagaaccagttgtaaatttgttgaagttcctaaagatagcacataccacaggaataccatatcatccagaaagtcagggtattgtagaaagggcaaataggaccttgaaagagaagattcaaagtcatagagacaacactcaaacactggaggcagctttacaacttgctctcattacttgtaacaaagggagggaaagtatgggaggacagacaccatgggaagtatttatcactaatcaagcacaagtaatacatgagaaacttttactacagcaagcacaatcctccaaaaaattttgtttttacaaaatccctggtgaacatgattggaagggacctactagggtgctgtggaagggtgatggtgcagtagtagttaatgatgaaggaaagggaataattgctgtaccattaaccaggactaagttactaataaaaccaaattga

SEQ ID NO: 28 - DNA, 385 bp, Artificial Sequence
  Description of Artificial Sequence pONY3.1
atgggagaccctttgacatggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatccaatcatgatag

SEQ ID NO: 29 - DNA, 385 bp, Artificial Sequence
  Description of Artificial Sequence pONY3.2opti
atgggcgatcccctcacctggtccaaagccctgaaaaaactggaaaaagtcaccgttcagggtagccaaaagcttaccacaggcaattgcaactgggcattgtccctggtggatcttttccacgacactaatttcgttaaggagaaagattggcaactcagagacgtgatccccctcttggaggacgtgacccaaacattgtctgggcaggagcgcgaagctttcgagcgcacctggtgggccatcagcgcagtcaaaatggggctgcaaatcaacaacgtggttgacggtaaagctagctttcaactgctccgcgctaagtacgagaaaaaaaccgccaacaagaaacaatccgaacctagcgaggagtacccaatcatgatag

SEQ ID NO: 30 - DNA, 12 bp, Human immunodeficiency virus
atgggtgcgaga

SEQ ID NO: 31 - DNA, 12 bp, Human immunodeficiency virus
gatgaggattag

SEQ ID NO: 32 - DNA, 12 bp, Artificial Sequence
  Description of Artificial Sequence gagpol-SYNgp
atgggcgcccgc

SEQ ID NO: 33 - DNA, 12 bp, Artificial Sequence
  Description of Artificial Sequence gagpol-SYNgp
gatgaggattag

SEQ ID NO: 34 - DNA, 12 bp, Human immunodeficiency virus
atgagagtgaag

SEQ ID NO: 35 - DNA, 12 bp, Human immunodeficiency virus
gctttgctataa

SEQ ID NO: 36 - DNA, 12 bp, Artificial Sequence
  Description of Artificial Sequence synGP160mn
atgagggtgaag

SEQ ID NO: 37 - DNA, 12 bp, Artificial Sequence
  Description of Artificial Sequence synGP160mn
gcgctgctgtaa